(19) United States
(12) Patent Application Publication
(10) Pub. No.: US 2025/0135022 A1
(43) Pub. Date: May 1, 2025

(54) METHODS FOR THE USE OF A B7-H3 ANTIBODY-DRUG CONJUGATE IN COMBINATION WITH A PD-1 X CTLA-4 BISPECIFIC MOLECULE

(71) Applicant: MACROGENICS, INC., Rockville, MD (US)

(72) Inventors: Scott KOENIG, Rockville, MD (US); Gerry Chester BOHAC, Rockville, MD (US); Bradley James SUMROW, Rockville, MD (US); Deryk LOO, Rockville, MD (US); Juniper A. SCRIBNER, Rockville, MD (US); Angela Joubert JAMES, Rockville, MD (US)

(21) Appl. No.: 18/836,888

(22) PCT Filed: Feb. 9, 2023
(86) PCT No.: PCT/US2023/062265

§ 371 Date: Aug. 8, 2024

Related U.S. Application Data
(60) Provisional application No. 63/331,370, filed on Apr. 15, 2022.
(60) Provisional application No. 63/308,903, filed on Feb. 10, 2022.

(51) Int. Cl. A61K 47/68; A61K 39/395; A61P 35/00

(52) U.S. Cl. CPC A61K 47/6849; A61K 39/3955; A61K 47/6803; A61K 47/6889; A61P 35/00

(57) ABSTRACT

The present disclosure is directed in part to dosing regimens for administering a humanized anti-B7-H3 antibody conjugated to a duocarmycin moiety (a “B7-H3-ADC”) for the treatment of cancer, particularly a cancer associated with expression of B17-H3. The disclosure in part concerns the use of such B7-H3-ADC in combination with a bispecific molecule capable of binding to PD-1 and CTLA-4 (“PD-1×CTLA-4 bispecific molecule”). The disclosure in pant concerns the use of such B7-H3-ADC in combination with lorigerlimab for the treatment of cancer. The disclosure in part concerns the use of MGC018 in combination with lorigerlimab for the treatment of cancer. The disclosure is directed in part to the use of such molecules, and to the use of pharmaceutical compositions and pharmaceutical kits that contain such molecules and that facilitate the use of such dosing regimens in the treatment of cancer.

### FIELD OF THE DISCLOSURE

The present disclosure pertains in part to dosing regimens for administering a humanized anti-B7-H3 antibody conjugated to a duocarmycin moiety (a “B7-H3-ADC”) for the treatment of cancer, including for example a cancer associated with expression of B7-H3. The disclosure in part concerns the use of such a B7-H3-ADC in combination with a bispecific molecule capable of binding to PD-1 and CTLA-4 (“PD-1×CTLA-4 bispecific molecule”). The disclosure in part concerns the use of such B7-H3-ADC in combination with lorigerlimab for the treatment of cancer. The disclosure in part concerns the use of MGC018 in combination with lorigerlimab for the treatment of cancer. The disclosure is directed in part to the use of such molecules, and to the use of pharmaceutical compositions and pharmaceutical kits that contain such molecules and that facilitate the use of such dosing regimens in the treatment of cancer.

### BACKGROUND OF THE DISCLOSURE

### I. B7-H3

B7-H3 is a member of the B7-CD28 Superfamily and is expressed on antigen-presenting cells. B7-H3 is unique in that the major human form contains two extracellular tandem IgV-IgC domains (i.e., IgV-IgC-IgV-IgC) (Collins, M. et al. (2005) “The B7 Family Of Immune-Regulatory Ligands,” Genome Biol. 6:223.1-223.7). B7-H3 is not expressed on resting B or T cells, monocytes, or dendritic cells, but it is induced on dendritic cells by IFN-γ and on monocytes by GM-CSF (Sharpe, A. H. et al. (2002) “The B7-CD28 Superfamily,” Nature Rev. Immunol. 2:116-126). The mode of action of B7-H3 is complex, and the protein is reported to mediate both T Cell co-stimulation and co-inhibition (Hofmeyer, K. et al. (2008) “The Contrasting Role Of B7-H3,” Proc. Natl. Acad. Sci. (U.S.A.) 105(30):10277-10278; Martin-Orozco, N. et al. (2007) “Inhibitory Costimulation And Anti-Tumor Immunity,” Semin. Cancer Biol. 17(4):288-298. B7-H3 binds to an unidentified receptor(s) to mediate co-inhibition of T cells. In addition, B7-H3, through interactions with unknown receptor(s) is an inhibitor for NK-cells and osteoblastic cells (Hofmeyer, K. et al. (2008) “The Contrasting Role Of B7-H3,” Proc. Natl. Acad. Sci. (U.S.A.) 105(30):10277-10278).

B7-H3 is expressed on a variety of cancer cells (e.g., neuroblastoma, gastric, ovarian, non-small cell lung cancers, etc., see, e.g., Modak, S., et al. (2001) “Monoclonal antibody 8H9 targets a novel cell surface antigen expressed by a wide spectrum of human solid tumors,” Cancer Res 61:4048-54) and cultured cancer stem-like cells. Several independent studies have shown that human malignant tumor cells exhibit a marked increase in expression of B7-H3 protein and that this increased expression was associated with increased disease severity (Tekle, C., et al. (2012) “B7-H3 Contributes To The Metastatic Capacity Of Melanoma Cells By Modulation Of Known Metastasis-Associated Genes,” Int. J. Cancer 130:2282-90; Wang, L., et al. (2013) “B7-H3 Mediated Tumor Immunology: Friend Or Foe?,” Int. J. Cancer 134(12):2764-2771), suggesting that B7-H3 is exploited by tumors as an immune evasion pathway (Hofmeyer, K. et al. (2008) “The Contrasting Role Of B7-H3,” Proc. Natl. Acad. Sci. (U.S.A.) 105(30):10277-10278).

The role of B7-H3 in inhibiting the immune system and the increased expression of B7-H3 on human tumors has suggested that this molecule might serve as a therapeutic target for the treatment of cancer. The use of anti-B7-H3 antibodies and other molecules that modulate B7-H3 expression to treat tumors and/or up-modulate an immune response has been proposed (see, Loo, D. et al. (2012) “Development of an Fc-Enhanced Anti-B7-H3 Monoclonal Antibody with Potent Antitumor Activity,” Clin Cancer Res; 18: 3834-3845; Ahmed, M. et al. (2015) “Humanized Affinity-Matured Monoclonal Antibody 8H9 Has Potent Anti-Tumor Activity and Binds to FG Loop of B7-H3,” J. Biol. Chem. 290: 30018-30029; Nagase-Zembutsu, A. et al. (2016) “Development of DS-5573a: A novel afucosylated monoclonal antibody directed at B7-H3 with potent antitumor activity,” Cancer Sci. 2016, doi: 10.1111/cas.12915; see also, U.S. Pat. Nos. 7,279,567, 7,527,969, 7,718,774, 8,779,098, 8,802,091, US Patent Publication Nos. 2002/0168762; 2008/0081346, 2008/0116219, 2013/0078234, 2015/0274838, PCT Publications Nos. WO 2009/073533; WO 2008/066691; WO 2006/016276; WO 2008/116219; WO 2001/094413, WO 2002/32375, WO 2004/093894, WO 2006/016276, WO 2008/116219, and WO 2011/109400.

### II. Cell-Mediated Immune Responses

The immune response is tightly controlled by co-stimulatory and co-inhibitory ligands and receptors often referred to as “immune checkpoints” (Chen et al., (2013) “Molecular Mechanisms of T Cell Co-Stimulation And Co-Inhibition,” Nature Rev. Immunol. 13:227-242; Pardoll, D. M., (2012) “The Blockade Of Immune Checkpoints In Cancer Immunotherapy,” Nat. Rev. Cancer 12(4):252-264). These molecules provide a balanced network of positive and negative signals that regulate immune responses to provide protection against infection and cancer. Some cancer cells are able to escape the immune system by engendering a state of T cell exhaustion in which T cells are exposed to persistent antigen and/or inflammatory signals (Wherry E. J. (2010) “T Cell Exhaustion,” Nat. Immunol. 12(6):492-499). Among the immune checkpoint molecules involved in T cell exhaustion are Programmed Death-1 (“PD-1”) and Cytotoxic T-lymphocyte associated protein-4 (“CTLA-4”) (Wherry, J. E. (2015) “Molecular And Cellular Insights Into T Cell Exhaustion,” Nat. Rev. Immunol. 15(8):486-499).

Bispecific molecules binding to both PD-1 and CTLA-4 allow for great flexibility in the design and engineering in various applications, providing enhanced avidity to multimeric antigens, the cross-linking of differing antigens, and directed targeting to specific cell types relying on the presence of both target antigens. The use of PD-1×CTLA-4 bispecific molecules in the treatment of cancer has been proposed and PD-1×CTLA-4 bispecific molecules have been described for example in WO 2014/209804; WO 2017/218707; WO 2017/193032; WO 2019/094637; and US 2019/0185569. In particular, tetravalent PD-1×CTLA-4 bispecific molecules are described in WO 2017/106061 and WO 2022/026306.

### SUMMARY OF THE DISCLOSURE

The present disclosure pertains in part to dosing regimens for administering a B7-H3-ADC for the treatment of cancer, including for example a cancer associated with expression of B7-H3. The disclosure in certain aspects can include administration of a single dose or a fractionated dose (i.e., in two or more separate administrations) of a B7-H3-ADC. The disclosure in certain aspects concerns the use of such B7-H3-ADC in combination with a PD-1×CTLA-4 bispecific molecule for the treatment of cancer. The dosing regimens for administering a B7-H3-ADC in combination with a PD-1×CTLA-4 bispecific molecule for the treatment of cancer, can include administration at regular dosing intervals or intermittent dosing intervals. In certain aspects, the dosing regimens for administering a B7-H3-ADC in combination with a PD-1×CTLA-4 bispecific molecule for the treatment of cancer, can include administration of a single dose or a fractionated dose (i.e., in two or more separate administrations) of a B7-H3-ADC. In certain aspects, the administration of a B7-H3-ADC and a PD-1×CTLA-4 bispecific molecule can be simultaneous or sequential in any order. The disclosure in certain aspects is directed to the use of such molecules, and to the use of pharmaceutical compositions and pharmaceutical kits that contain such molecules and that facilitate the use of such dosing regimens in the treatment of cancer.

In detail, the disclosure provides a method of treating a cancer in a subject in need thereof, comprising administering a B7-H3-ADC for the treatment of cancer, including for example a cancer associated with expression of B7-H3, in a dosage regimen that can include administration of a fractionated dose (i.e., in two or more separate administrations) of a B7-H3-ADC. In detail, the disclosure provides a method of treating a cancer in a subject in need thereof, comprising administering an anti-B7-H3 antibody-drug conjugate (B7-H3-ADC) and a PD-1×CTLA-4 bispecific molecule to said subject, wherein the B7-H3-ADC comprises the formula:

Ab-(LM)ₘ-(D)ₙ,
wherein:
Ab is a humanized B7-H3 antibody or B7-H3 binding fragment thereof that binds to B7-H3 and comprises:
   (i) the CDRL1 sequence RASESIYSYLA (SEQ ID NO:22), the CDRL2 sequence NTKTLPE (SEQ ID NO:23) and the CDRL3 sequence QHHYGTPPWT (SEQ ID NO:24) in its Variable Light Chain (VL) domain, and
   (ii) the CDRH1 sequence SYGMS (SEQ ID NO:25), the CDRH2 sequence TINSGGSNTYY PDSLKG (SEQ ID NO:26) and the CDRH3 sequence HDGGAMDY (SEQ ID NO:27) in its Variable Heavy Chain (VH) domain;
D is a cytotoxic duocarmycin moiety;
LM comprises at least one bond or a Linker Molecule that covalently links Ab and D;
m is an integer between 0 and n and denotes the number of bonds or Linker Molecules of said B7-H3-ADC, except when LM is a bond, m is not 0;
and
n is an integer between 1 and 10 and denotes the number of cytotoxic duocarmycin moieties covalently linked to said B7-H3-ADC molecule.

The disclosure further provides an embodiment of such method, wherein the Ab comprises:
(i) a humanized Variable Light Chain (VL) domain comprising the amino acid sequence of SEQ ID NO:17; and
(ii) a humanized Variable Heavy Chain (VH) domain comprising the amino acid sequence of SEQ ID NO:18.

The disclosure further provides an embodiment of such method, wherein the Ab further comprises an Fc of a human IgG1, IgG2, IgG3, or IgG4.

The disclosure further provides an embodiment of such method, wherein the Ab further comprises an Fc Domain of a human IgG1.

The disclosure further provides an embodiment of such method, wherein the Fc Domain is a variant Fc Domain that comprises:
(a) one or more amino acid modifications that reduces the affinity of the variant Fc Domain for an FcγR; and/or
(b) one or more amino acid modifications that enhances the serum half-life of the variant Fc Domain.

The disclosure further provides an embodiment of such method, wherein the modifications that reduces the affinity of the variant Fc Domain for an FcγR comprise the substitution of L234A; L235A; or L234A and L235A, wherein the numbering is that of the EU index as in Kabat.

The disclosure further provides an embodiment of such method, wherein the modifications that that enhances the serum half-life of the variant Fc Domain comprise the substitution of M252Y; M252Y and S254T; M252Y and T256E; M252Y, S254T and T256E; or K288D and H435K, wherein the numbering is that of the EU index as in Kabat.

The disclosure further provides an embodiment of such method, wherein at least one of the LM is a Linker Molecule.

The disclosure further provides an embodiment of such method, wherein the LM Linker Molecule is a peptidic linker.

The disclosure further provides an embodiment of such method, wherein the peptidic linker is a valine-citrulline dipeptide linker.

The disclosure further provides an embodiment of such method, wherein the LM Linker Molecule further comprises a self-eliminating spacer between the cleavable linker and D.

The disclosure further provides an embodiment of such method, wherein the self-eliminating spacer comprises a para-aminobenzyloxycarbonyl moiety.

The disclosure further provides an embodiment of such method, wherein the LM Linker Molecule further comprises a maleimide linker moiety between the cleavable linker and Ab.

The disclosure further provides an embodiment of such method, wherein LM is represented by the formula:

[V—(W)ₖ—(X)₁-A]
whereby said B7-H3-ADC is represented by the formula:

Ab-[V—(W)ₖ—(X)₁-A]-D
wherein:
V is a cleavable linker,
(W)ₖ—(X)₁-A is an elongated, self-eliminating spacer system, that self-eliminates via a 1,(4+2n)-elimination,
W and X are each a 1,(4+2n) electronic cascade spacer, being the same or different,
A is either a spacer group of formula (Y)ₘ, wherein Y is a 1,(4+2n) electronic cascade spacer, or a group of formula U, being a cyclisation elimination spacer, k, l and m are independently an integer of 0 (included) to 5 (included),
n is an integer of 0 (included) to 10 (included),
with the provisos that:
when A is (Y)ₘ: then k+l+m≥1, and
if k+l+m=l, then n>l;
when A is U: then k+1≥1.
W, X, and Y are independently selected from compounds having the formula:

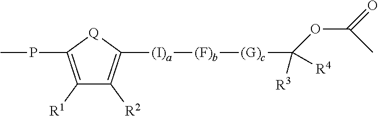

or the formula:

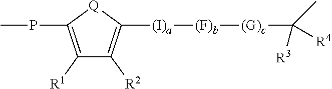

wherein: Q is —R⁵C═CR⁶—, S, O, NR⁵, —R⁵C═N—, or —N═CR⁵—
P is NR⁷, O or S
a, b, and c are independently an integer of 0 (included) to 5 (included);
I, F and G are independently selected from compounds having the formula:

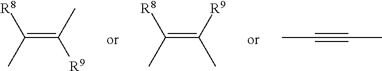

wherein R¹, R², R³, R⁴, R⁵, R⁶, R⁷, R⁸, and R⁹ independently represent H, C₁₋₆ alkyl, C₃₋₂₀ heterocyclyl, C₅₋₂₀ aryl, C₁₋₆ alkoxy, hydroxy (OH), amino (NH₂), mono-substituted amino (NRₓH), di-substituted amino (NRₓ¹Rₓ²), nitro (NO₂), halogen, CF₃, CN, CONH₂, SO₂Me, CONHMe, cyclic C₁₋₅ alkylamino, imidazolyl, C₁₋₆ alkylpiperazinyl, morpholino, thiol (SH), thioether (SRₓ), tetrazole, carboxy (COOH), carboxylate (COORₓ), sulphoxy (S(═O)₂OH), sulphonate (S(═O)₂ORₓ), sulphonyl (S(═O)₂Rₓ), sulphixy (S(═O)OH), sulphinate (S(═O)ORₓ), sulphinyl (S(═O)Rₓ), phosphonooxy (OP(═O)(OH)₂), and phosphate (OP(═O)(ORₓ)₂), where Rₓ, Rₓ¹ and Rₓ² are independently selected from a C₁₋₆ alkyl group, a C₃₋₂₀ heterocyclyl group or a C₅₋₂₀ aryl group, two or more of the substituents R¹, R², R³, R⁴, R⁵, R⁶, R⁷, R⁸, or R⁹ optionally being connected to one another to form one or more aliphatic or aromatic cyclic structures;
U is selected from compounds having the formula:

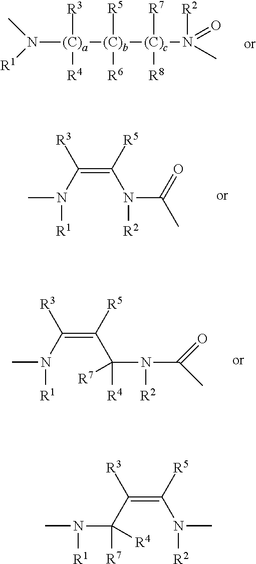

wherein:
a, b and c are independently selected to be an integer of 0 or 1; provided that a+b+c=2 or 3;
R¹ and/or R² independently represent H, C₁₋₆ alkyl, the alkyl being optionally substituted with one or more of the following groups: hydroxy (OH), ether (ORₓ), amino (NH₂), mono-substituted amino (NRₓH), disubstituted amino (NRₓ¹Rₓ²), nitro (NO₂), halogen, CF₃, CN, CONH₂, SO₂Me, CONHMe, cyclic C₁₋₅ alkylamino, imidazolyl, C₁₋₆ alkylpiperazinyl, morpholino, thiol (SH), thioether (SRₓ), tetrazole, carboxy (COOH), carboxylate (COORₓ), sulphoxy (S(═O)₂OH), sulphonate (S(═O)₂₀Rₓ), sulphonyl (S(═O)₂Rₓ), sulphixy (S(═O)OH), sulphinate (S(═O)ORₓ), sulphinyl (S(═O)Rₓ), phosphonooxy (OP(═O)(OH)₂), and phosphate (OP(═O)(ORₓ)₂), where Rₓ, Rₓ¹ and Rₓ² are selected from a C₁₋₆ alkyl group, a C₃₋₂₀ heterocyclyl group or a C₅₋₂₀ aryl group; and
R³, R⁴, R⁵, R⁶, R⁷ and R⁸ independently represent H, C₁₋₆ alkyl, C₃₋₂₀ heterocyclyl, C₅₋₂₀ aryl, C₁₋₆ alkoxy, hydroxy (OH), amino (NH₂), mono-substituted amino (NRₓH), disubstituted amino (NRₓ¹Rₓ²), nitro (NO₂), halogen, CF₃, CN, CONH₂, SO₂Me, CONHMe, cyclic C₁₋₅ alkylamino, imidazolyl, C₁₋₆ alkylpiperazinyl, morpholino, thiol (SH), thioether (SRₓ), tetrazole, carboxy (COOH), carboxylate (COORₓ), sulphoxy (S(═O)₂OH), sulphonate (S(═O)₂ORₓ), sulphonyl (S(═O)₂Rₓ), sulphixy (S(═O)OH), sulphinate (S(═O)ORₓ), sulphinyl (S(═O)Rₓ), phosphonooxy (OP(═O)(OH)₂), and phosphate (OP(═O)(ORₓ)₂), where Rₓ, Rₓ¹ and Rₓ² are selected from a C₁₋₆ alkyl group, a C₃₋₂₀ heterocyclyl group or a C₅₋₂₀ aryl group, and two or more of the substituents R¹, R², R³, R⁴, R⁵, R⁶, R⁷, or R⁸ are optionally connected to one another to form one or more aliphatic or aromatic cyclic structures.

The disclosure further provides an embodiment of such method, wherein the LM linker molecule comprises:
(1) p-aminobenzyloxycarbonyl-p-aminobenzyloxycarbonyl;
(2) p-aminobenzyloxycarbonyl-p-aminobenzyloxycarbonyl-p-aminobenzyloxycarbonyl;
(3) p-ammocinnamyloxycarbonyl;
(4) p-aminocinnamyloxycarbonyl-p-aminobenzyloxycarbonyl;
(5) p-amino-benzyloxycarbonyl-p-aminocinnamyloxycarbonyl;
(6) p-aminocinnamyloxycarbonyl-p-aminocinnamyloxycarbonyl;
(7) p-aminophenylpentadienyloxycarbonyl;
(8) p-aminophenylpentadienyloxycarbonyl-p-aminocinnamyloxycarbonyl;
(9) p-aminophenylpentadienyloxycarbonyl-p-aminobenzyloxycarbonyl;
(10) p-aminophenylpentadienyloxycarbonyl-p-aminophenylpentadienyloxycarbonyl;
(11) p-aminobenzyloxycarbonyl(methylamino)ethyl(methylamino) carbonyl;
(12) p-aminocinnamyloxycarbonyl(methylamino)ethyl(methylamino) carbonyl;
(13) p-aminobenzyloxycarbonyl-p-aminobenzyloxycarbonyl(methylamino) ethyl(methylamino)carbonyl;
(14) p-aminocinnamyloxycarbonyl-p-aminobenzyloxycarbonyl (methylamino)ethyl(methylamino)carbonyl;
(15) p-aminobenzyloxycarbonyl-p-aminocinnamyloxycarbonyl (methylamino)ethyl(methylamino)-carbonyl;
(16) p-aminocinnamyloxycarbonyl-p-aminocinnamyloxycarbonyl (methylamino)ethyl(methylamino)carbonyl;
(17) p-aminobenzyloxycarbonyl-p-aminobenzyl;
(18) p-aminobenzyloxycarbonyl-p-aminobenzyloxycarbonyl-p-aminobenzyl;
(19) p-aminocinnamyl;
(20) p-aminocinnamyloxycarbonyl-p-aminobenzyl;
(21) p-aminobenzyloxycarbonyl-p-aminocinnamyl;
(22) p-amino-cinnamyloxycarbonyl-p-aminocinnamyl;
(23) p-aminophenylpentadienyl;
(24) p-aminophenylpentadienyloxycarbonyl-p-aminocinnamyl;
(25) p-aminophenylpentadienyloxycarbonyl-p-aminobenzyl; or
(26) p-aminophenylpentadienyloxycarbonyl-p-aminophenylpentadienyl.

The disclosure further provides an embodiment of such method, wherein the LM Linker Molecule is conjugated to the side chain of an amino acid of a polypeptide chain of the Ab and binds the Ab to a molecule of the cytotoxic duocarmycin moiety D.

The disclosure further provides an embodiment of such method, wherein the cytotoxic duocarmycin moiety D comprises a duocarmycin cytotoxin selected from the group consisting of: duocarmycin A, duocarmycin B1, duocarmycin B2, duocarmycin C₁, duocarmycin C2, duocarmycin D, duocarmycin SA, CC-1065, adozelesin, bizelesin, carzelesin (U-80244), seco-duocarmycin (seco-DUBA) and spiro-duocarmycin (spiro-DUBA or DUBA).

The disclosure further provides an embodiment of such method, wherein the cytotoxic duocarmycin moiety D comprises seco-DUBA.

The disclosure further provides an embodiment of such method, wherein the LM Linker Molecule is covalently linked to the Ab via reduced inter-chain disulfides.

The disclosure further provides an embodiment of such method, wherein the Ab comprises:
(i) a light chain comprising the Variable Light Chain (VL) domain comprising the amino acid sequence of SEQ ID NO:17 and a CL Kappa Domain of SEQ ID NO:1; and
(ii) a heavy chain comprising the Variable Heavy Chain (VH) domain comprising the amino acid sequence of SEQ ID NO:18, a CH1 Domain of SEQ ID NO:3, a Hinge Domain of SEQ ID NO:5 and a Fc Domain comprising a CH2-CH3 Domain of SEQ ID NO:8;
the D comprises seco-DUBA; and
the LM comprises a Linker Molecule comprising a maleimide linker moiety, a
valine-citrulline dipeptide linker, and a para-aminobenzyloxycarbonyl moiety.

The disclosure further provides an embodiment of such method, wherein the Ab comprises:
(i) a light chain comprising the amino acid sequence of SEQ ID NO:19; and
(ii) a heavy chain comprising the amino acid sequence of SEQ ID NO:20;
the D comprises seco-DUBA; and
the LM comprises a Linker Molecule comprising a maleimide linker moiety, a valine-citrulline dipeptide linker, and a para-aminobenzyloxycarbonyl moiety.

The disclosure further provides an embodiment of such method, wherein the B7-H3-ADC is administered at a therapeutically effective or prophylactically effective dose of about 1 mg/kg to about 3 mg/kg every 3 weeks.

The disclosure further provides an embodiment of such method, wherein the B7-H3-ADC is administered at a therapeutically effective or prophylactically effective dose of about 2 mg/kg to about 3 mg/kg every 3 weeks.

The disclosure further provides an embodiment of such method, wherein the B7-H3-ADC is administered at a therapeutically effective or prophylactically effective dose of about 1 mg/kg to about 3 mg/kg every 4 weeks.

The disclosure further provides an embodiment of such method, wherein the B7-H3-ADC is administered at a therapeutically effective or prophylactically effective dose of about 2 mg/kg to about 3 mg/kg every 4 weeks.

The disclosure further provides an embodiment of such method, wherein the B7-H3-ADC is administered as a single dose.

The disclosure further provides an embodiment of such method, wherein the B7-H3-ADC is administered as a fractionated dose in two or more separate administrations.

The disclosure further provides an embodiment of such method, wherein the fractionated dose comprises two separate administrations administered within about 7±2 days of each other.

The disclosure further provides an embodiment of such method, wherein the fractionated dose comprises two separate administrations administered within a 3-week cycle.

The disclosure further provides an embodiment of such method, wherein the fractionated dose comprises two separate administrations administered within a 4-week cycle.

The disclosure further provides an embodiment of such method, wherein the B7-H3-ADC is administered before the administration of the PD-1×CTLA-4 bispecific molecule on days when both the B7-H3-ADC and the PD-1×CTLA-4 bispecific molecule are administered.

The disclosure further provides an embodiment of such method, wherein the PD-1×CTLA-4 is administered before the administration of the B7-H3-ADC bispecific molecule on days when both the B7-H3-ADC and the PD-1×CTLA-4 bispecific molecule are administered.

The disclosure further provides an embodiment of such method, wherein the PD-1×CTLA-4 bispecific molecule is administered at least about 15-30 minutes after the B7-H3-ADC is administered.

The disclosure further provides an embodiment of such method, wherein the the B7-H3-ADC bispecific molecule is administered at least about 15-30 minutes after the the PD-1×CTLA-4 bispecific molecule is administered.

The disclosure further provides an embodiment of such method, wherein the PD-1×CTLA-4 bispecific molecule is administered at least about 15 minutes after the B7-H3-ADC is administered.

The disclosure further provides an embodiment of such method, wherein the the B7-H3-ADC bispecific molecule is administered at least about 15 minutes after the the PD-1×CTLA-4 bispecific molecule is administered.

The disclosure further provides an embodiment of such method, wherein the PD-1×CTLA-4 bispecific molecule is administered at least about 30 minutes after the B7-H3-ADC is administered.

The disclosure further provides an embodiment of such method, wherein the the B7-H3-ADC bispecific molecule is administered at least about 30 minutes after the the PD-1×CTLA-4 bispecific molecule is administered.

The disclosure further provides an embodiment of such method, wherein the PD-1×CTLA-4 bispecific molecule is selected from the group consisting of lorigerlimab, MEDI5752, vudalimab, and cadonilimab.

The disclosure further provides an embodiment of such method, wherein the PD-1×CTLA-4 bispecific molecule is lorigerlimab.

The disclosure further provides an embodiment of such method, wherein the lorigerlimab is administered at a dose of about 1 mg/kg, about 3 mg/kg or about 6 mg/kg every 3 weeks.

The disclosure further provides an embodiment of such method, wherein the lorigerlimab is administered at a dose of about 6 mg/kg every 3 weeks.

The disclosure further provides an embodiment of such method, wherein the lorigerlimab is administered at a dose of about 1 mg/kg, about 3 mg/kg or about 6 mg/kg every 4 weeks.

The disclosure further provides an embodiment of such method, wherein the lorigerlimab is administered at a dose of about 6 mg/kg every 4 weeks.

The disclosure further provides an embodiment of such method, wherein the B7-H3-ADC is administered at a dose of about 1 mg/kg.

The disclosure further provides an embodiment of such method, wherein the B7-H3-ADC is administered at a dose of about 1.25 mg/kg.

The disclosure further provides an embodiment of such method, wherein the B7-H3-ADC is administered at a dose of about 1.5 mg/kg.

The disclosure further provides an embodiment of such method, wherein the B7-H3-ADC is administered at a dose of about 1.75 mg/kg.

The disclosure further provides an embodiment of such method, wherein the B7-H3-ADC is administered at a dose of about 2 mg/kg.

The disclosure further provides an embodiment of such method, wherein the B7-H3-ADC is administered at a dose of about 2.1 mg/kg.

The disclosure further provides an embodiment of such method, wherein the B7-H3-ADC is administered at a dose of about 2.2 mg/kg.

The disclosure further provides an embodiment of such method, wherein the B7-H3-ADC is administered at a dose of about 2.25 mg/kg.

The disclosure further provides an embodiment of such method, wherein the B7-H3-ADC is administered at a dose of about 2.3 mg/kg.

The disclosure further provides an embodiment of such method, wherein the B7-H3-ADC is administered at a dose of about 2.4 mg/kg.

The disclosure further provides an embodiment of such method, wherein the B7-H3-ADC is administered at a dose of about 2.5 mg/kg.

The disclosure further provides an embodiment of such method, wherein the B7-H3-ADC is administered at a dose of about 2.6 mg/kg.

The disclosure further provides an embodiment of such method, wherein the B7-H3-ADC is administered at a dose of about 2.7 mg/kg.

The disclosure further provides an embodiment of such method, wherein the B7-H3-ADC is administered at a dose of about 2.75 mg/kg.

The disclosure further provides an embodiment of such method, wherein the B7-H3-ADC is administered at a dose of about 3 mg/kg.

The disclosure further provides an embodiment of such method, wherein the B7-H3-ADC is administered by intravenous (IV) infusion.

The disclosure further provides an embodiment of such method, wherein the IV infusion of the B7-H3-ADC is over a period of at least about 60-120 minutes.

The disclosure further provides an embodiment of such method, wherein the IV infusion of the B7-H3-ADC is over a period of at least about 60 minutes.

The disclosure further provides an embodiment of such method, wherein the IV infusion of the B7-H3-ADC is over a period of at least about 75 minutes.

The disclosure further provides an embodiment of such method, wherein the IV infusion of the B7-H3-ADC is over a period of at least about 120 minutes.

The disclosure further provides an embodiment of such method, wherein the PD-1×CTLA-4 bispecific molecule is administered by IV infusion.

The disclosure further provides an embodiment of such method, wherein the IV infusion of the PD-1×CTLA-4 bispecific molecule is over a period of at least about 30-120 minutes.

The disclosure further provides an embodiment of such method, wherein the IV infusion of the PD-1×CTLA-4 bispecific molecule is over a period of at least about 30 minutes.

The disclosure further provides an embodiment of such method, wherein the IV infusion of the PD-1×CTLA-4 bispecific molecule is over a period of at least about 45 minutes.

The disclosure further provides an embodiment of such method, wherein the IV infusion of the PD-1×CTLA-4 bispecific molecule is over a period of at least about 60 minutes.

The disclosure further provides an embodiment of such method, wherein the cancer is selected from the group consisting of: an adrenal gland cancer, an AIDS-associated cancer, an alveolar soft part sarcoma, an astrocytic tumor, an anal cancer, squamous cell carcinoma of the anal canal (SCAC), a bladder cancer, a bone cancer, a brain and spinal cord cancer, a metastatic brain tumor, a B-cell cancer, a breast cancer, a HER2+ breast cancer, triple negative breast cancer (TNBC), a carotid body tumors, a cervical cancer, a chondrosarcoma, a chordoma, a chromophobe renal cell carcinoma, a clear cell carcinoma, a colon cancer, a colorectal cancer (CRC), a non-microsatellite instability high colorectal cancer (non-MSI-H CRC), a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, a gastric cancer, a gestational trophoblastic disease, a germ cell tumor, a head and neck cancer, a glioblastoma, a hematological malignancy, a hepatocellular carcinoma, an islet cell tumor, a Kaposi's Sarcoma, a kidney cancer, a leukemia, an acute myeloid leukemia, a liposarcoma/malignant lipomatous tumor, a dedifferentiated liposarcoma, a liver cancer, a lymphoma, a lung cancer, a non-small-cell lung cancer (NSCLC), a medulloblastoma, a melanoma, a cutaneous melanoma, a meningioma, a mesothelioma pharyngeal cancer, a multiple endocrine neoplasia, a multiple myeloma, a myelodysplastic syndrome, a myxofibrosarcoma, a neuroblastoma, a neuroendocrine tumors, an ovarian cancer, a pancreatic cancer, a papillary thyroid carcinoma, a parathyroid tumor, a pediatric cancer, a peripheral nerve sheath tumor, a phaeochromocytoma, a pituitary tumor, a prostate cancer, a metastatic castration resistant prostate cancer (mCRPC), a posterior uveal melanoma, a renal cell cancer, a renal cell carcinoma (RCC), a renal metastatic cancer, a rhabdoid tumor, a rhabdomyosarcoma, a sarcoma, a skin cancer, a small round blue cell tumor of childhood, a neuroblastoma, a soft tissue sarcoma, a pleomorphic undifferentiated sarcoma, a squamous cell cancer, a squamous cell cancer of the head and neck (SCCHN), a stomach cancer, a synovial sarcoma, a testicular cancer, a thymic carcinoma, a thymoma, a thyroid cancer, a thyroid metastatic cancer, and a uterine cancer.

The disclosure further provides an embodiment of such method, wherein the cancer is selected from the group consisting of: anal cancer, SCAC, breast cancer, TNBC, cervical cancer, colorectal cancer, non-microsatellite instability high colorectal cancer (non-MSI-H CRC), head and neck cancer, kidney cancer, renal cell carcinoma, liver cancer, hepatocellular carcinoma, lung cancer, NSCLC, melanoma, cutaneous melanoma, posterior uveal melanoma, ovarian cancer, pancreatic cancer, prostate cancer, mCRPC, soft tissue sarcoma, dedifferentiated liposarcoma, myxofibrosarcoma, pleomorphic undifferentiated sarcoma, synovial sarcoma, squamous cell cancer, and SCCHN.

The disclosure further provides an embodiment of such method, wherein the cancer is prostate cancer.

The disclosure further provides an embodiment of such method, wherein the prostate cancer is mCRPC.

The disclosure further provides an embodiment of such method, wherein the cancer is liver cancer.

The disclosure further provides an embodiment of such method, wherein the liver cancer is hepatocellular carcinoma.

The disclosure further provides an embodiment of such method, wherein the cancer is kidney cancer.

The disclosure further provides an embodiment of such method, where the kidney cancer is renal cell carcinoma.

The disclosure further provides an embodiment of such method, wherein the cancer is ovarian cancer.

The disclosure further provides an embodiment of such method, where the cancer is pancreatic cancer.

The disclosure further provides an embodiment of such method, wherein the cancer is anal cancer.

The disclosure further provides an embodiment of such method, wherein the anal cancer is SCAC.

The disclosure further provides an embodiment of such method, wherein the cancer is a squamous cell cancer.

The disclosure further provides an embodiment of such method, wherein the squamous cell cancer is SCCHN.

The disclosure further provides an embodiment of such method, wherein the cancer is breast cancer.

The disclosure further provides an embodiment of such method, wherein the breast cancer is TNBC.

The disclosure further provides an embodiment of such method, wherein the cancer is melanoma.

The disclosure further provides an embodiment of such method, wherein the melanoma is a cutaneous melanoma or a posterior uveal melanoma.

The disclosure further provides an embodiment of such method, wherein the cancer is lung cancer.

The disclosure further provides an embodiment of such method, wherein the lung cancer is NSCLC.

The disclosure further provides an embodiment of such method, wherein the cancer is cervical cancer.

The disclosure further provides an embodiment of such method, wherein the cancer is colorectal cancer.

The disclosure further provides an embodiment of such method, wherein the colorectal cancer is non-MSI-H CRC.

The disclosure further provides an embodiment of such method, wherein the cancer is a soft tissue sarcoma.

The disclosure further provides an embodiment of such method, wherein the soft tissue sarcoma is dedifferentiated liposarcoma, myxofibrosarcoma, pleomorphic undifferentiated sarcoma, or synovial sarcoma.

The disclosure further provides an embodiment of such method, wherein the cancer expresses B7-H3.

The disclosure further provides an embodiment of such method, further comprising administering a therapeutically or prophylactically effective amount of one or more additional therapeutic agents or chemotherapeutic agents.

The disclosure further provides an embodiment of such method, wherein the chemotherapeutic agent is a platinum-based chemotherapeutic agent.

The disclosure further provides an embodiment of such method, wherein the chemotherapeutic agent is a taxane.

The disclosure further provides an embodiment of such method, wherein the subject in need thereof is a human.

### BRIEF DESCRIPTION OF THE DRAWINGS

FIG. 1A provides a schematic representation of MGC018 showing mechanism of release of the active toxin (DUBA). MGC018 is an ADC composed of a humanized monoclonal IgG1 antibody covalently linked to a restricted number of linker-drug moieties, containing a seco-duocarmycin derivative. The humanized monoclonal IgG1 antibody recognizes human B7-H3. The linker-drug contains a cleavable linker and the prodrug seco-duocarmycin-hydroxybenzamide-azaindole (seco-DUBA). Upon receptor-mediated internalization of MGC018, the linker is cleaved by lysosomal proteases at the dipeptide valine-citrulline (vc) motif The prodrug (seco-DUBA) then spontaneously rearranges through cyclization to form the active toxin (DUBA) which can then bind and alkylate DNA based on the mechanism of action of the drug. FIG. 1B provides a schematic showing representative covalently bonded tetravalent diabody, such as a PD-1×CTLA-4 bispecific diabody, having four epitope-binding sites composed of two pairs of polypeptide chains (i.e., four polypeptide chains in all). One polypeptide of each pair has an E-coil Heterodimer-Promoting Domain and the other polypeptide of each pair has a K-coil Heterodimer-Promoting Domain. As shown, a cysteine residue may be present in a linker and/or in the Heterodimer-Promoting Domain. One polypeptide of each pair possesses a linker comprising a cysteine (which linker may comprise all or a portion of a hinge region) and CH2 and/or CH3 Domain, such that the associated chains form all or part of an Fc Region. VL and VH Domains that recognize the same epitope are shown using the same shading or fill pattern. The VL and VH Domains recognize different epitopes and the resulting molecule possesses four epitope-binding sites and is bispecific and bivalent with respect to each bound epitope.

FIG. 2 shows the results of a study comparing the efficacy of a single dose versus a fractionated dose of MGC018, to mediate in vivo cytotoxicity against subcutaneously implanted Calu-6 lung cancer cells in a CD-1 nude mouse model. The tumor growth curves are presented for mice treated intravenously with a single dose of 12 mg/kg of MGC018 (QW×1, dashed arrow) or with a fractionated dose of MGC018 (0.3 mg/kg, 1 mg/kg, or 3 mg/kg) administered once a week for 4 weeks (QW×4, solid arrows). Vehicle was used as a negative control and administered only on day 21.

FIGS. 3A-3F shows the results of a study comparing the efficacy of a single dose versus a fractionated dose of MGC018, to mediate in vivo cytotoxicity against subcutaneously implanted A375.S2 melanoma cells (FIGS. 3A and 3B), Calu-6 lung cancer cells (FIGS. 3C and 3D), or MDA-MD-468 triple negative breast cancer cells (FIGS. 3E and 3F) in a CES1c knockout mouse model. The tumor growth curves are presented for mice treated intravenously with a single dose of MGC018 (1 mg/kg or 3 mg/kg; QW×1) (FIGS. 3A, 3C, and 3E) or with a fractionated dose of MGC018 (0.3 mg/kg or 1 mg/kg) (FIGS. 3B, 3D, and 3F) administered once a week for 4 weeks (QW×4). Vehicle was used as a negative control and administered only on the first dosing day.

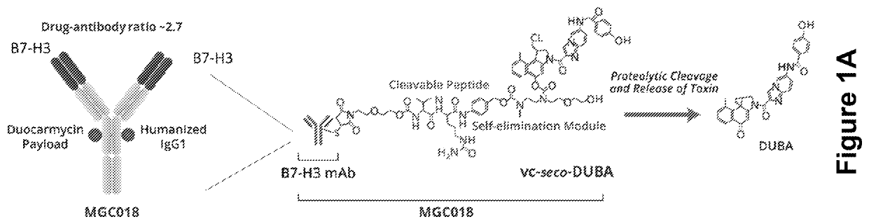

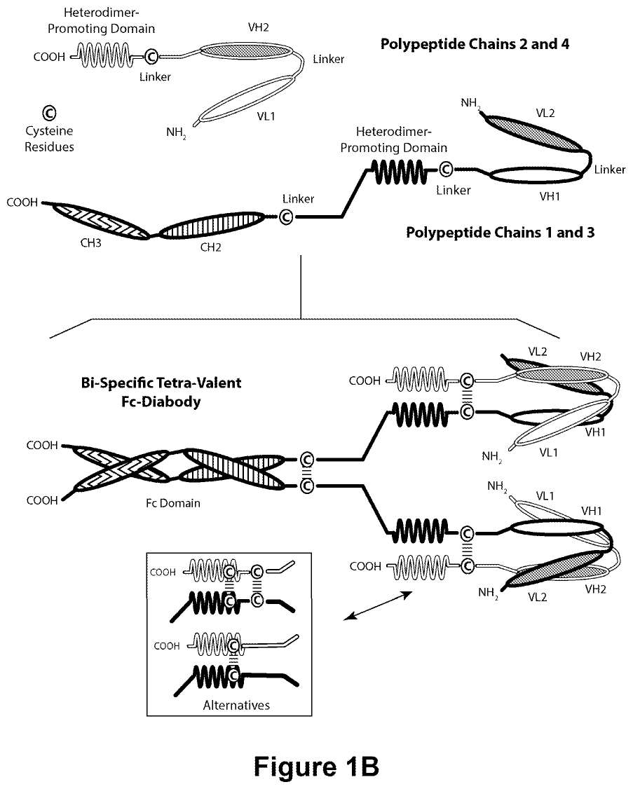

### DETAILED DESCRIPTION OF THE DISCLOSURE

The present disclosure pertains in part to dosing regimens for administering a B7-H3-ADC for the treatment of cancer, including for example a cancer associated with expression of B7-H3. The disclosure in certain aspects can include administration of a single dose or a fractionated dose (i.e., in two or more separate administrations) of a B7-H3-ADC. The disclosure in certain aspects concerns the use of such B7-H3-ADC in combination with a PD-1×CTLA-4 bispecific molecule for the treatment of cancer. The dosing regimens for administering a B7-H3-ADC in combination with a PD-1×CTLA-4 bispecific molecule for the treatment of cancer, can include administration at regular dosing intervals or intermittent dosing intervals. In certain aspects, the dosing regimens for administering a B7-H3-ADC in combination with a PD-1×CTLA-4 bispecific molecule for the treatment of cancer, can include administration of a single dose or a fractionated dose (i.e., in two or more separate administrations) of a B7-H3-ADC. In certain aspects, the administration of a B7-H3-ADC and a PD-1×CTLA-4 bispecific molecule can be simultaneous or sequential in any order. In certain aspects, the B7-H3-ADC is MGC018. In certain aspects, the PD-1×CTLA-4 bispecific molecule is lorigerlimab. The disclosure in certain aspects is directed to the use of such molecules, and to the use of pharmaceutical compositions and pharmaceutical kits that contain such molecules and that facilitate the use of such dosing regimens in the treatment of cancer.

### I. Antibodies and Their Binding Domains

The antibodies of the present disclosure are immunoglobulin molecules capable of specific binding to a target, such as a carbohydrate, polynucleotide, lipid, polypeptide, etc., through at least one antigen recognition site, located in the Variable Domain of the immunoglobulin molecule. A B7-H3-ADC thus comprises an antibody that binds to B7-H3. As used herein, the terms “antibody” and “antibodies” refer to monoclonal antibodies, multispecific antibodies, human antibodies, humanized antibodies, synthetic antibodies, chimeric antibodies, polyclonal antibodies, camelized antibodies, single-chain Fvs (scFv), single-chain antibodies, Fab fragments, F(ab′) fragments, disulfide-linked bispecific Fvs (sdFv), intrabodies, and epitope-binding fragments of any of the above. In particular, the term “antibody” includes immunoglobulin molecules and immunologically active fragments of immunoglobulin molecules, i.e., molecules that contain an epitope-binding site. Immunoglobulin molecules can be of any type (e.g., IgG, IgE, IgM, IgD, IgA and IgY), class (e.g., IgG₁, IgG₂, IgG₃, IgG₄, IgA₁ and IgA₂) or subclass. Antibodies are capable of “immunospecifically binding” to a polypeptide or protein or a non-protein molecule (or of binding to such molecule in an “immunospecific manner”) due to the presence on such molecule of a particular domain or moiety or conformation (an “epitope”). An epitope-containing molecule may have immunogenic activity, such that it elicits an antibody production response in an animal; such molecules are termed “antigens”.

As used herein, an antibody, diabody or other epitope-binding molecule is said to “immunospecifically” bind a region of another molecule (i.e., an epitope) if it reacts or associates more frequently, more rapidly, with greater duration and/or with greater affinity with that epitope relative to alternative epitopes. For example, an antibody that immunospecifically binds to a viral epitope is an antibody that binds this viral epitope with greater affinity, avidity, more readily, and/or with greater duration than it immunospecifically binds to other viral epitopes or non-viral epitopes. It is also understood by reading this definition that, for example, an antibody (or moiety or epitope) that immunospecifically binds to a first target may or may not specifically or preferentially bind to a second target. As such, “immunospecific binding” does not necessarily require (although it can include) exclusive binding. Generally, but not necessarily, reference to binding means “immunospecific” binding. Two molecules are said to be capable of binding to one another in a “physiospecific” manner, if such binding exhibits the specificity with which receptors bind to their respective ligands.

The term “monoclonal antibody” refers to a homogeneous antibody population wherein the monoclonal antibody is comprised of amino acids (naturally occurring or non-naturally occurring) that are involved in the selective binding of an antigen. Monoclonal antibodies are highly specific, being directed against a single epitope (or antigenic site). The term “monoclonal antibody” encompasses not only intact monoclonal antibodies and full-length monoclonal antibodies, but also fragments thereof (such as Fab, Fab′, F(ab′)₂, Fv, etc.), single-chain (scFv) binding molecules, mutants thereof, fusion proteins comprising an antibody portion, humanized monoclonal antibodies, chimeric monoclonal antibodies, and any other modified configuration of the immunoglobulin molecule that comprises an antigen recognition site of the required specificity and the ability to bind to an antigen. It is not intended to be limited as regards to the source of the antibody or the manner in which it is made (e.g., by hybridoma, phage selection, recombinant expression, transgenic animals, etc.). The term includes whole immunoglobulins as well as the fragments etc. described above under the definition of “antibody.” Methods of making monoclonal antibodies are known in the art. One method which may be employed is the method of Kohler, G. et al. (1975) “Continuous Cultures Of Fused Cells Secreting Antibody Of Predefined Specificity,” Nature 256:495-497 or a modification thereof. Typically, monoclonal antibodies are developed in mice, rats or rabbits. The antibodies are produced by immunizing an animal with an immunogenic amount of cells, cell extracts, or protein preparations that contain the desired epitope. The immunogen can be, but is not limited to, primary cells, cultured cell lines, cancerous cells, proteins, peptides, nucleic acids, or tissue. Alternatively, existing monoclonal antibodies and any other equivalent antibodies that are immunospecific for a desired pathogenic epitope can be sequenced and produced recombinantly by any means known in the art. In one embodiment, such an antibody is sequenced and the polynucleotide sequence is then cloned into a vector for expression or propagation. The sequence encoding the antibody of interest may be maintained in a vector in a host cell and the host cell can then be expanded and frozen for future use. The polynucleotide sequence of such antibodies may be used for genetic manipulation to generate the monospecific or multispecific (e.g., bispecific, trispecific and tetraspecific) molecules as well as an affinity optimized, a chimeric antibody, a humanized antibody, and/or a caninized antibody, to improve the affinity, or other characteristics of the antibody. The general principle in humanizing an antibody involves retaining the basic sequence of the antigen-binding portion of the antibody, while swapping the non-human remainder of the antibody with human antibody sequences and are well known in the art. See, for example, U.S. Pat. Nos. 4,816,567; 5,807,715; 5,866,692; 5,997,867; 6,054,297; 6,180,377; 6,331,415; and European Patent No. 519,596.

Natural antibodies (such as IgG antibodies) are composed of two “Light Chains” complexed with two “Heavy Chains.” Each Light Chain contains a Variable Domain (“VL”) and a Constant Domain (“CL”). Each Heavy Chain contains a Variable Domain (“VH”), three Constant Domains (“CH1,” “CH2” and “CH3”), and a “Hinge” Region (“H”) located between the CH1 and CH2 Domains. The basic structural unit of naturally occurring immunoglobulins (e.g., IgG) is thus a tetramer having two light chains and two heavy chains, usually expressed as a glycoprotein of about 150,000 Da. The amino-terminal (“N-terminal”) portion of each chain includes a Variable Domain of about 100 to 110 or more amino acids primarily responsible for antigen recognition. The carboxy-terminal (“C-terminal”) portion of each chain defines a constant region, with light chains having a single Constant Domain and heavy chains usually having three Constant Domains and a Hinge Domain. Thus, the structure of the light chains of an IgG molecule is n-VL-CL-c and the structure of the IgG heavy chains is n-VH-CH1-H-CH2-CH3-c (where n and c represent, respectively, the N-terminus and the C-terminus of the polypeptide).

### A. Characteristics of Antibody Variable Domains

The Variable Domains of an IgG molecule consist of the complementarity determining regions (“CDR”), which contain the residues in contact with epitope, and non-CDR segments, referred to as framework segments (“FR”), which in general maintain the structure and determine the positioning of the CDR loops so as to permit such contacting (although certain framework residues may also contact antigen). Thus, the VL and VH Domains have the structure n-FR1-CDR1-FR2-CDR2-FR3-CDR3-FR4-c. The amino acid sequences of the CDRs determine whether an antibody will be able to bind to a particular epitope. Interaction of an antibody light chain with an antibody heavy chain and, in particular, interaction of their VL and VH Domains, forms an epitope-binding site of the antibody.

Amino acids from the Variable Domains of the mature heavy and light chains of immunoglobulins are designated by the position of an amino acid in the chain. Kabat (SEQUENCES OF PROTEINS OF IMMUNOLOGICAL INTEREST, 5th Ed. Public Health Service, NH1, MD (1991)) described numerous amino acid sequences for antibodies, identified an amino acid consensus sequence for each subgroup, and assigned a residue number to each amino acid, and the CDRs and FRs are identified as defined by Kabat (it will be understood that CDR_{H}1 as defined by Chothia, C. & Lesk, A. M. ((1987) “Canonical structures for the hypervariable regions of immunoglobulins,” J. Mol. Biol. 196:901-917) begins five residues earlier). Kabat's numbering scheme is extendible to antibodies not included in his compendium by aligning the antibody in question with one of the consensus sequences in Kabat by reference to conserved amino acids. This method for assigning residue numbers has become standard in the field and readily identifies amino acids at equivalent positions in different antibodies, including chimeric or humanized variants. For example, an amino acid at position 50 of a human antibody light chain occupies the equivalent position to an amino acid at position 50 of a mouse antibody light chain. The positions within the VL and VH Domains at which the CDRs commence and end are thus well defined and can be ascertained by inspection of the sequences of the VL and VH Domains (see, e.g., Martin, C. R. (2010) “Protein Sequence and Structure Analysis of Antibody Variable Domains,” In: ANTIBODY ENGINEERING VOL. 2 (Kontermann, R. and Dübel, S. (eds.), Springer-Verlag Berlin Heidelberg, Chapter 3 (pages 33-51)).

Polypeptides that are (or may serve as) the first, second and third CDR of the Light Chain of an antibody are herein respectively designated as: CDR_{L}1 Domain, CDR_{L}2 Domain, and CDR_{L}3 Domain. Similarly, polypeptides that are (or may serve as) the first, second and third CDR of the Heavy Chain of an antibody are herein respectively designated as: CDR_{H}1 Domain, CDR_{H}2 Domain, and CDR_{H}3 Domain. Thus, the terms CDR_{L}1 Domain, CDR_{L}2 Domain, CDR_{L}3 Domain, CDR_{H}1 Domain, CDR_{H}2 Domain, and CDR_{H}3 Domain are directed to polypeptides that when incorporated into a protein cause that protein to be able to bind to a specific epitope regardless of whether such protein is an antibody having light and heavy chains or is a diabody or a single-chain binding molecule (e.g., an scFv, a BiTe, etc.), or is another type of protein. Accordingly, as used herein, the term “epitope-binding fragment” denotes a fragment of a molecule capable of immunospecifically binding to an epitope. An epitope-binding fragment may contain any 1, 2, 3, 4, or 5 the CDR Domains of an antibody, or may contain all 6 of the CDR Domains of an antibody and, although capable of immunospecifically binding to such epitope, may exhibit an immunospecificity, affinity or selectivity toward such epitope that differs from that of such antibody. Generally, an epitope-binding fragment will contain all 6 of the CDR Domains of such antibody. An epitope-binding fragment of an antibody may be a single polypeptide chain (e.g., an scFv), or may comprise two or more polypeptide chains, each having an amino terminus and a carboxy terminus (e.g., a diabody, a Fab fragment, an Fab₂ fragment, etc.). Unless specifically noted, the order of domains of the protein molecules described herein is in the “N-terminal to C-Terminal” direction.

The disclosure particularly encompasses single-chain Variable Domain fragments (“scFv”) comprising a humanized anti-B7-H3-VL and/or VH Domain of this invention. Single-chain Variable Domain fragments comprise VL and VH Domains that are linked together using a short “Linker” peptide. Such Linkers can be modified to provide additional functions, such as to permit the attachment of a drug or to permit attachment to a solid support. The single-chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

### B. Characteristics of Antibody Constant Domains

### 1. Constant Domains of the Light Chain

As indicated above, each Light Chain of an antibody contains a Variable Domain (“VL”) and a Constant Domain (“CL”).

A representative CL Domain is a human IgG CL Kappa Domain. The amino acid sequence of a human CL Kappa Domain is (SEQ ID NO:1):

RTVAAPSVFI FPPSDEQLKS GTASVVCLLN NFYPREAKVQ
WKVDNALQSG NSQESVTEQD SKDSTYSLSS TLTLSKADYE
KHKVYACEVT HQGLSSPVTK SENRGEC

Another representative CL Domain is a human IgG CL Lambda Domain. The amino acid sequence of a human CL Lambda Domain is (SEQ ID NO:2):

QPKAAPSVTL FPPSSEELQA NKATLVCLIS DFYPGAVTVA
WKADSSPVKA GVETTPSKQS NNKYAASSYL SLTPEQWKSH
RSYSCQVTHE GSTVEKTVAP TECS

### 2. Constant Domains of the Heavy Chain

As indicated above, the heavy chains of an antibody may comprise CH1, Hinge Domain, CH2 and CH3 constant domains. The CH1 Domains of the two heavy chains of an antibody complex with the antibody's Light Chain's CL constant region and are attached to the heavy chains CH2 Domains via an intervening Hinge Domain.

A representative CH1 Domain is a human IgG1 CH1 Domain. The amino acid sequence of a human IgG1 CH1 Domain is (SEQ ID NO:3):

ASTKGPSVFP LAPSSKSTSG GTAALGCLVK DYFPEPVTVS
WNSGALTSGV HTFPAVLQSS GLYSLSSVVT VPSSSLGTQT
YICNVNHKPS NTKVDKRV

Another representative CH1 Domain is a human IgG4 CH1 Domain. The amino acid sequence of a human IgG4 CH1 Domain is (SEQ ID NO:4):

ASTKGPSVFP LAPCSRSTSE STAALGCLVK DYFPEPVTVS
WNSGALTSGV HTFPAVLQSS GLYSLSSVVT VPSSSLGTKT
YTCNVDHKPS NTKVDKRV

A representative Hinge Domain is a human IgG1 Hinge Domain. The amino acid sequence of a human IgG1 Hinge Domain is (SEQ ID NO:5): EPKSCDKTHTCP PCP

Another representative Hinge Domain is a human IgG4 Hinge Domain. The amino acid sequence of a human IgG4 Hinge Domain is (SEQ ID NO:6): ESKYGPPCPSCP. An IgG4 Hinge Domain may comprise a stabilizing mutation such as the S228P substitution. The amino acid sequence of a S228P-stabilized human IgG4 Hinge Domain is (SEQ ID NO:7): ESKYGPPCPPCP.

The CH2 and CH3 Domains of the two heavy chains of an antibody interact to form an “Fc Domain,” which is a domain that is recognized by cellular Fc Receptors, including but not limited to Fc gamma Receptors (FcγRs). As used herein, the term “Fc Domain” is used to define a C-terminal region of an IgG heavy chain. An Fc Domain is said to be of a particular IgG isotype, class or subclass if its amino acid sequence is most homologous to that isotype relative to other IgG isotypes. In addition to their known uses in diagnostics, antibodies have been shown to be useful as therapeutic agents.

The amino acid sequence of the CH2-CH3 Domain of a representative human IgG1 is (SEQ ID NO:8):

```
231 240 250 260 270 280
APELLGGPSV FLFPPKPKDT LMISRTPEVT CVVVDVSHED PEVKENWYVD
                  290 300 310 320 330
GVEVHNAKTK PREEQYNSTY RVVSVLTVLH QDWLNGKEYK CKVSNKALPA
                  340 350 360 370 380
PIEKTISKAK GQPREPQVYT LPPSREEMTK NQVSLTCLVK GFYPSDIAVE
                  390 400 410 420 430
WESNGQPENN YKTTPPVLDS DGSFFLYSKL TVDKSRWQQG NVFSCSVMHE
                  440 447
ALHNHYTQKS LSLSPGX
```

as numbered by the EU index as set forth in Kabat, wherein X is a lysine (K) or is absent.

The amino acid sequence of the CH2-CH3 Domain of a representative human IgG4 is (SEQ ID NO:9):

```
231 240 250 260 270 280
APEFLGGPSV FLFPPKPKDT LMISRTPEVT CVVVDVSQED PEVQFNWYVD
                  290 300 310 320 330
GVEVHNAKTK PREEQFNSTY RVVSVLTVLH QDWLNGKEYK CKVSNKGLPS
                  340 350 360 370 380
SIEKTISKAK GQPREPQVYT LPPSQEEMTK NQVSLTCLVK GFYPSDIAVE
                  390 400 410 420 430
WESNGQPENN YKTTPPVLDS DGSFFLYSRL TVDKSRWQEG NVFSCSVMHE
                  440 447
ALHNHYTQKS LSLSLGX
```

as numbered by the EU index as set forth in Kabat, wherein X is a lysine (K) or is absent.

Throughout the present specification, the numbering of the residues in the constant region of an IgG heavy chain is that of the EU index as in Kabat et al., SEQUENCES OF PROTEINS OF IMMUNOLOGICAL INTEREST, 5^{th} Ed. Public Health Service, NH1, MD (1991), expressly incorporated herein by reference. The term “EU index as in Kabat” refers to the numbering of the constant domains of human IgG1 EU antibody.

Polymorphisms have been observed at a number of different positions within antibody constant regions (e.g., Fc positions, including but not limited to positions 270, 272, 312, 315, 356, and 358 as numbered by the EU index as set forth in Kabat), and thus slight differences between the presented sequence and sequences in the prior art can exist. Polymorphic forms of human immunoglobulins have been well-characterized. At present, 18 Gm allotypes are known: G1m (1, 2, 3, 17) or G1m (a, x, f, z), G2m (23) or G2m (n), G3m (5, 6, 10, 11, 13, 14, 15, 16, 21, 24, 26, 27, 28) or G3m (b1, c3, b3, b0, b3, b4, s, t, g1, c5, u, v, g5) (Lefranc, et al., “The Human IgG Subclasses: Molecular Analysis Of Structure, Function And Regulation.” Pergamon, Oxford, pp. 43-78 (1990); Lefranc, G. et al., 1979, Hum. Genet.: 50, 199-211). It is specifically contemplated that the antibodies of the present disclosure may incorporate any allotype, isoallotype, or haplotype of any immunoglobulin gene, and are not limited to the allotype, isoallotype or haplotype of the sequences provided herein. Furthermore, in some expression systems the C-terminal amino acid residue (bolded above) of the CH3 Domain may be post-translationally removed. Accordingly, the C-terminal residue of the CH3 Domain is an optional amino acid residue. Specifically encompassed by the instant disclosure is a B7-H3-ADC lacking the C-terminal residue of the CH3 Domain. Also specifically encompassed by the instant disclosure are such constructs comprising the C-terminal lysine residue of the CH3 Domain.

The present disclosure particularly encompasses B7-H3-ADCs comprising anti-B7-H3 Variable Domains (i.e., VL and/or VH Domains) that immunospecifically bind to an epitope of a human B7-H3 polypeptide. Such B7-H3-ADCs are capable of immunospecifically binding to human B7-H3. As used herein such B7-H3 Variable Domains are referred to as “anti-B7-H3-VL” and “anti-B7-H3-VH,” respectively.

### II. Modification of the Fc Domain

The Fc Domain of the Fc Domain-containing molecules (e.g., antibodies and diabodies) may be either a complete Fc Domain (e.g., a complete IgG Fc Domain) or only a fragment of an Fc Domain. Optionally, the Fc Domain of the Fc Domain-containing molecules lack the C-terminal lysine amino acid residue.

In traditional immune function, the interaction of antibody-antigen complexes with cells of the immune system results in a wide array of responses, ranging from effector functions such as antibody dependent cytotoxicity, mast cell degranulation, and phagocytosis to immunomodulatory signals such as regulating lymphocyte proliferation and antibody secretion. All of these interactions are initiated through the binding of the Fc Domain of antibodies or immune complexes to specialized cell surface receptors (singularly referred to as an “Fc gamma receptor,” “FcγR,” and collectively as “FcγRs”) found on the surfaces of multiple types of immune system cells (e.g., B lymphocytes, follicular dendritic cells, natural killer cells, macrophages, neutrophils, eosinophils, basophils and mast cells). The diversity of cellular responses triggered by antibodies and immune complexes results from the structural heterogeneity of the three Fc receptors: FcγRI (CD64), FcγRII (CD32), and FcγRIII (CD16). FcγRI (CD64), FcγRIIA (CD32A) and FcγRIII (CD16) are activating (i.e., immune system enhancing) receptors; FcγRIIB (CD32B) is an inhibiting (i.e., immune system dampening) receptor. In addition, interaction with the neonatal Fc Receptor (FcRn) mediates the recycling of IgG molecules from the endosome to the cell surface and release into the blood. The amino acid sequence of a representative wild-type IgG1 (SEQ ID NO:8) and a representative wild-type IgG4 (SEQ ID NO:9) are presented above.

Modification of the Fc Domain may lead to an altered phenotype, for example altered serum half-life, altered stability, altered susceptibility to cellular enzymes or altered effector function. Accordingly, in certain embodiments, the Fc Domain of the Fc Domain-containing molecules may be an engineered variant Fc Domain. Although the Fc Domain of the Fc Domain-containing molecules may possess the ability to bind to one or more Fc receptors (e.g., FcγR(s)), in particular such variant Fc Domain will have altered binding to FcγRIA (CD64), FcγRIIA (CD32A), FcγRIIB (CD32B), FcγRIIIA (CD16a), or FcγRIIIB (CD16b) (relative to the binding exhibited by a wild-type Fc Domain), e.g., will have enhanced binding to an activating receptor and/or will have substantially reduced or no ability to bind to inhibitory receptor(s). Thus, the Fc Domain of the Fc Domain-containing molecules may include some or all of the CH2 Domain and/or some or all of the CH3 Domain of a complete Fc Domain, or may comprise a variant CH2 and/or a variant CH3 sequence (that may include, for example, one or more insertions and/or one or more deletions with respect to the CH2 or CH3 domains of a complete Fc Domain). Such Fc Domains may comprise non-Fc polypeptide portions, or may comprise portions of non-naturally complete Fc Domains, or may comprise non-naturally occurring orientations of CH2 and/or CH3 Domains (such as, for example, two CH2 domains or two CH3 domains, or in the N-terminal to C-terminal direction, a CH3 Domain linked to a CH2 Domain, etc.).

In certain embodiments, the Fc Domains of the binding molecules exhibit decreased (or substantially no) binding to FcγRIA (CD64), FcγRIIA (CD32A), FcγRIIB (CD32B), FcγRIIIA (CD16a) or FcγRIIIB (CD16b) (relative to the binding exhibited by the wild-type IgG1 Fc Domain (SEQ ID NO:8). In certain embodiments, the binding molecules comprise an IgG Fc Domain that exhibits reduced ADCC effector function. In a such embodiments, the CH2-CH3 Domains of binding molecules include any 1, 2, 3, or 4 of the substitutions: L234A, L235A, D265A, N297Q, and N297G. In another embodiment, the CH2-CH3 Domains contain an N297Q substitution, an N297G substitution, L234A and L235A substitutions or a D265A substitution, as these mutations abolish FcR binding. Alternatively, a CH2-CH3 Domain of a naturally occurring Fc Domain that inherently exhibits decreased (or substantially no) binding to FcγRIIIA (CD16a) and/or reduced effector function (relative to the binding and effector function exhibited by the wild-type IgG1 Fc Domain (SEQ ID NO:8)) is utilized. In a specific embodiment, the binding molecules comprise an IgG4 Fc Domain (SEQ ID:NO:9). When an IgG4 Fc Domain is utilized, the instant disclosure also encompasses the introduction of a stabilizing mutation, such as the Hinge Domain S228P substitution described herein (see, e.g., SEQ ID NO:7).

The serum half-life of proteins comprising Fc Domains may be increased by increasing the binding affinity of the Fc Domain for FcRn. The term “half-life” as used herein means a pharmacokinetic property of a molecule that is a measure of the mean survival time of the molecules following their administration. Half-life can be expressed as the time required to eliminate fifty percent (50%) of a known quantity of the molecule from a subject's body (e.g., a human patient or other mammal) or a specific compartment thereof, for example, as measured in serum, i.e., circulating half-life, or in other tissues. In general, an increase in half-life results in an increase in mean residence time (MRT) in circulation for the molecule administered. Modifications capable of increasing the half-life of an Fc Domain-containing molecule are known in the art and include, for example M252Y, S254T, T256E, and combinations thereof. For example, see the modifications described in U.S. Pat. Nos. 6,277,375; 7,083,784; 7,217,797; and 8,088,376; U.S. Publication Nos. 2002/0147311; 2007/0148164; and 2011/0081347.

In one embodiment, a non-limiting example of a PD-1×CTLA-4 bispecific molecule, lorigerlimab, comprises a variant IgG4 Fc Region, wherein such variant IgG4 Fc Region comprises a substitution at position 252 with tyrosine, 254 with threonine, and 256 with glutamate (252Y, 254T and 256E), wherein such numbering is that of the EU index as in Kabat.

A variant IgG4 sequence for the CH2 and CH3 Domains comprising the M252Y/S254T/T256E substitutions is (SEQ ID NO:14):

APEFLGGPSV FLFPPKPKDT LYITREPEVT CVVVDVSQED PEVQFNWYVD
GVEVHNAKTK PREEQFNSTY RVVSVLTVLH QDWLNGKEYK CKVSNKGLPS
SIEKTISKAK GQPREPQVYT LPPSQEEMTK NQVSLTCLVK GFYPSDIAVE
WESNGQPENN YKTTPPVLDS DGSFFLYSRL TVDKSRWQEG NVESCSVMHE
ALHNHYTQKS LSLSLGX

wherein, X is a lysine (K) or is absent.

### III. Anti-B7-H3 Antibody mAb-A

A representative anti-B7-H3 antibody, designated “mAb-A,” was isolated from hybridoma cells that had been produced through immunization with cells expressing human B7-H3, with a B7-H3 polypeptide or a peptide epitope thereof. Antibody mAb-A was humanized.

Antibody mAb-A was found to be cross-reactive with B7-H3 of cynomolgus monkeys. The amino acid sequences of the VL and VH Domains of mAb-A are provided below. The B7-H3-ADC possess all 3 of the CDR_{HS} of the VH Domain, all 3 of the CDR_{LS} of the VL Domain, and optionally the entire VH and VL Domains of humanized monoclonal antibody mAb-A (“hmAb-A”).

A. Murine Anti-B7-H3 Antibody mAb-A

The amino acid sequence of the VL Domain of the murine anti-B7-H3 antibody mAb-A (SEQ ID NO:15) is shown below (CDR_{L} residues are shown underlined):

DIQMTQSPAS LSVSVGETVT ITCRASESIY SYLAWYQQKQ GKSPQLLVYN
TKTLPEGVPS RESGSGSGTQ FSLKINSLQP EDFGRYYCQH HYGTPPWTFG
GGTNLEIK

The amino acid sequence of the VH Domain of anti-B7-H3 mAb-A (SEQ ID NO:16) is shown below (CDR_{H} residues are shown underlined).

EVQQVESGGD LVKPGGSLKL SCAASGFTES SYGMSWVRQT PDKRLEWVAT
INSGGSNTYY PDSLKGRFTI SRDNAKNTLY LQMRSLKSED TAMYYCARHD
GGAMDYWGQG TSVTVSS

B. Humanized Anti-B7-H3 Antibody hmAb-A

The Variable Domains of the anti-B7-H3 antibody mAb-A were humanized to generate a humanized mAb-A (“hmAb-A”). In in some instances alternative humanized Variable Domains were generated to optimize binding activity and/or to remove antigenic epitopes and/or to remove potentially labile amino acid residues.

The amino acid sequence of the VL Domain of hmAb-A (SEQ ID NO:17) is shown below (CDR_{L} residues are shown underlined):

DIQMTQSPSS LSASVGDRVT ITCRASESIY SYLAWYQQKP GKAPKLLVYN
TKTLPEGVPS RESGSGSGTD FTLTISSLQP EDFATYYCQH HYGTPPWTFG
QGTRLEIK

The amino acid sequence of a Light Chain of hmAb-A comprising the VL of Domain of hmAb-A and a CL Kappa Domain (SEQ ID NO: 19) is shown below:

| | | | | |
|---|---|---|---|---|
| DIQMTQSPSS | LSASVGDRVT | ITCRASESIY | SYLAWYQQKP | GKAPKLLVYN |
| TKTLPEGVPS | RESGSGSGTD | FTLTISSLQP | EDFATYYCQH | HYGTPPWTFG |
| QGTRLEIKRT | VAAPSVFIFP | PSDEQLKSGT | ASVVCLLNNF | YPREAKVQWK |
| VDNALQSGNS | QESVTEQDSK | DSTYSLSSTL | TLSKADYEKH | KVYACEVTHQ |
| GLSSPVTKSF | NRGEC | | | |

In SEQ ID NO:19, amino acid residues 1-108 correspond to the VL Domain of hmAb-A (SEQ ID NO:17), and amino acid residues 109-215 correspond to the Light Chain kappa constant region (SEQ ID NO:1).

The amino acid sequence of the VH Domain of hmAb-A (SEQ ID NO:18) is shown below (CDR_{H} residues are shown underlined).

EVQLVESGGG LVKPGGSLRL SCAASGFTES SYGMSWVRQA
PGKGLEWVAT INSGGSNTYY PDSLKGRFTI SRDNAKNSLY
LQMNSLRAED TAVYYCARHD GGAMDYWGQG TTVTVSS

The amino acid sequence of a Heavy Chain comprising the VH Domain of hmAb-A and IgG1 CH1-H-CH2-CH3 Domains (SEQ ID NO:20) is shown below:

| | | | | |
|---|---|---|---|---|
| EVQLVESGGG | LVKPGGSLRL | SCAASGFTES | SYGMSWVRQA | PGKGLEWVAT |
| INSGGSNTYY | PDSLKGRETI | SRDNAKNSLY | LQMNSLRAED | TAVYYCARHD |
| GGAMDYWGQG | TTVTVSSAST | KGPSVFPLAP | SSKSTSGGTA | ALGCLVKDYF |
| PEPVTVSWNS | GALTSGVHTF | PAVLQSSGLY | SLSSVVTVPS | SSLGTQTYIC |
| NVNHKPSNTK | VDKRVEPKSC | DKTHTCPPCP | APELLGGPSV | FLFPPKPKDT |
| LMISRTPEVT | CVVVDVSHED | PEVKENWYVD | GVEVHNAKTK | PREEQYNSTY |
| RVVSVLTVLH | QDWLNGKEYK | CKVSNKALPA | PIEKTISKAK | GQPREPQVYT |
| LPPSREEMTK | NQVSLTCLVK | GFYPSDIAVE | WESNGQPENN | YKTTPPVLDS |
| DGSFFLYSKL | TVDKSRWQQG | NVESCSVMHE | ALHNHYTQKS | LSLSPGX |

wherein, X is a lysine (K) or is absent.

In SEQ ID NO:20, amino acids 1-117 correspond to the VH Domain of hmAb-A (SEQ ID NO:18), amino acid residues 118-215 correspond to the IgG1 CH1 Domain (SEQ ID NO:3), amino acid residues 216-230 correspond to the IgG1 Hinge Domain (SEQ ID NO:5), and amino acid residues 231-447 correspond to the IgG1 CH2-CH3 Domain (SEQ ID NO:8). An N-linked glycosylation site is present at Kabat position 296 (shown underlined). The C-terminal residue “X” is a lysine (K) or is absent.

### IV. B7-H3-ADC

The present disclosure relates to the above-described anti-B7-H3 antibody hmAb-A conjugated to a cytotoxic drug, a “B7-H3-ADC”. Such B7-H3-ADC enhances the cytotoxicity of anti-B7-H3 therapy, particularly in the treatment of cancer. As indicated above, a B7-H3-ADC is represented by the formula:

Ab-(LM)ₘ-(D)ₙ,

wherein:
Ab is an antibody that binds to B7-H3 that comprises a humanized Variable Heavy Chain (VH) Domain and a humanized Variable Light Chain (VL) Domain, or is a B7-H3-binding fragment thereof, and;
D is a cytotoxic duocarmycin moiety;
LM is a bond or a Linker Molecule that covalently links Ab and D;
m is an integer between 0 and n and denotes the number of bonds or Linker Molecules of the B7-H3-ADC, except when LM is a bond, m is not 0;
and
n is an integer between 1 and 10 and denotes the number of cytotoxic duocarmycin moieties covalently linked to the B7-H3-ADC.

In certain embodiments, a B7-H3-ADC comprises a naturally occurring Fc Domain of the IgG1 isotype. Such Fc Domain lacks the C-terminal lysine residue of a CH3 Domain. In specific embodiments, a B7-H3-ADC will bind to a tumor cell expressing B7-H3 and will then be internalized into such cell through receptor-mediated endocytosis. Once inside a lysosome, a B7-H3-ADC may be degraded so as to thereby cause the release of the cytotoxic duocarmycin moiety inside the cell, resulting in cell death. As will be appreciated, the mechanism of action of cell death can vary based on the class of cytotoxic drug used (e.g., disruption of cytokinesis by tubulin polymerization inhibitors such as maytansines and auristatins, DNA damage by DNA interacting agents such as calicheamicins and duocarmycins), etc. Neighboring cancer cells may also be killed when free drug is released into the tumor environment by the dying cell in a process known as the bystander effect (Panowski, S. et al. (2014) “Site-Specific Antibody Drug Conjugates For Cancer Therapy,” mAbs 6(1):34-45; Kovtun, Y. V. et al. (2006) “Antibody-Drug Conjugates Designed To Eradicate Tumors With Homogeneous And Heterogeneous Expression Of The Target Antigen,” Cancer Res. 66:3214-3221).

### A. Linker Molecules of the Invention

The disclosure particularly contemplates such B7-H3-ADCs wherein LM is a Linker Molecule and is absent (i.e., m=0), and B7-H3-ADCs that possess more than one Linker Molecule LM (i.e., m is an integer from 2 through n, wherein n is an integer from 2 through 10), each of which Linker Molecule LM covalently links a cytotoxic duocarmycin moiety D to the Ab of such B7-H3-ADCs.

The disclosure further provides B7-H3-ADCs whose Ab are covalently linked to more than one Linker Molecule LM, wherein all such Linker Molecules are identical. The cytotoxic duocarmycin moieties D that are covalently linked to the Ab of such B7-H3-ADCs may all be identical or may include 2, 3, 4, or more independently different cytotoxic duocarmycin moieties D.

The disclosure further provides such B7-H3-ADCs whose Ab are covalently linked to more than one Linker Molecule LM, wherein all such Linker Molecules are not identical and may independently differ. The cytotoxic duocarmycin moieties D that are covalently linked to the Ab of such B7-H3-ADCs may all be identical or may include 2, 3, 4, or more independently different cytotoxic duocarmycin moieties D.

Humanized VH and VL Domains of antibodies that bind to human B7-H3, and human antibody Constant Domains that may be included in a B7-H3-ADC are provided above. As stated above, a B7-H3-ADC additionally comprise at least one cytotoxic duocarmycin moiety, which is covalently linked to an atom of a side chain of an amino acid residue of such VH Domain or VL Domain and/or Constant Domain, either directly, or via a Linker Molecule intercalated between the side chain atom and the duocarmycin moiety. The Linker Molecule may be a non-peptide molecule, or a molecule that comprises a non-peptide portion and a peptide portion, or it may be a molecule that is composed solely of amino acid residues. The amino acid residues of any such Linker Molecules may contain naturally occurring or non-naturally occurring amino acid residues, including D-versions of naturally occurring amino acid residues, p-acetylphenylalanine, selenocysteine, etc. Optionally, or additionally, particular residues having a desired side chain (e.g., a —CH₂—SH side chain, a —CH₂—OH side chain, a —CH(CH₂)—SH side chain, a —CH₂—CH₂—S—CH₃ side chain; a —CH₂—C(O)—NH₂ side chain, a —CH₂—CH₂—C(O)—NH₂ side chain, a —CH₂—C(O)OH— side chain, a CH₂—CH₂—C(O)OH— side chain, a —CH₂—CH₂—CH₂—CH₂—NH₂ side chain, a —CH₂—CH₂—CH₂—NH—C(NH₂)₂ side chain, an imidazole side chain, a benzyl side chain, a phenol side chain, an indole side chain, etc.) may be engineered into a B7-H3-ADC.

The Linker Molecule LM may be non-cleavable under physiologic conditions, for example composed of a hydrolytically stable moiety, for example, a thioether linker or a hindered disulfide linker. Hydrolytically stable linkers are substantially stable in water and do not react with water at useful pH values, including but not limited to, under physiological conditions for an extended period of time. In contrast, hydrolytically unstable or degradable linkers are degradable in water or in aqueous solutions, including for example, blood.

Alternatively, the Linker Molecule LM may be cleavable, or may contain a cleavable portion. Examples of such a cleavable portion includes an acid labile linker (e.g., a 4-(4′-acetylphenoxy) butanoic acid linker which forms a hydrazine bond), a cleavable disulfide linker (that is cleaved in the reducing intracellular environment), and a protease cleavable linker. Acid-labile linkers are designed to be stable at pH levels encountered in the blood, but become unstable and degrade when the low pH environment in lysosomes is encountered. Protease-cleavable linkers are also designed to be stable in blood/plasma, but rapidly release free drug inside lysosomes in cancer cells upon cleavage by lysosomal enzymes (Panowski, S. et al. (2014) “Site-Specific Antibody Drug Conjugates For Cancer Therapy,” mAbs 6(1):34-45). Alternatively, the Linker Molecule may be an enzyme-cleavable-substrate or contain an enzyme-cleavable-substrate, such as a cleavable peptide, (e.g., a cleavable dipeptide such as a valine-citrulline dipeptide para-aminobenzylalcohol linker (cAC10-mc-vc-PABA) which is selectively cleaved by lysosomal enzymes). Suitable cleavable linkers are known in the art, see, e.g., de Groot, Franciscus M. H., et al. (2002) “Design, Synthesis, and Biological Evaluation of a Dual Tumor-Specific Motive Containing Integrin-Targeted Plasmin-Cleavable Doxorubicin Prodrug,” Molecular Cancer Therapeutics, 1: 901-911; Dubowchik et al., (2002) “Doxorubicin Immunoconjugates Containing Bivalent, Lysosomally-Cleavable Dipeptide Linkages.” Bioorganic & Medicinal Chemistry Letters 12:1529-1532; U.S. Pat. Nos. 5,547,667; 6,214,345; 7,585,491; 7,754,681; 8,080,250; 8,461,117; and WO 02/083180.

Enzymatically unstable or degradable linkers can be employed. Such linkers are degraded by one or more enzymes. By way of example only, PEG and related polymers can include a degradable Linker Molecule(s) in the polymer backbone or in the linker group between the polymer backbone and one or more of the terminal functional groups of the polymer molecule. Such degradable Linker Molecule(s) include, but are not limited to, ester linkages formed by the reaction of PEG carboxylic acids or activated PEG carboxylic acids with alcohol groups on a biologically active agent, wherein such ester groups generally hydrolyze under physiological conditions to release the biologically active agent. Other hydrolytically degradable Linker Molecules include but are not limited to carbonate linkages; imine linkages resulting from reaction of an amine and an aldehyde; phosphate ester linkages formed by reacting an alcohol with a phosphate group; hydrazone linkages that are a reaction product of a hydrazide and an aldehyde; acetal linkages that are the reaction product of an aldehyde and an alcohol; orthoester linkages that are the reaction product of a formate and an alcohol; peptide linkages formed by an amine group, including but not limited to, at an end of a polymer such as PEG, and a carboxyl group of a peptide; and oligonucleotide linkages formed by a phosphoramidite group, including but not limited to, at the end of a polymer, and a 5′ hydroxyl group of an oligonucleotide.

In one embodiment, the Linker Molecule LM may be, or may comprise, a cleavable Linker Molecule, V—(W)ₖ—(X)₁-A, as disclosed in PCT Publication WO 02/083180, resulting in a B7-H3-ADC having the formula:

Ab-[V—(W)ₖ—(X)₁-A]-D

wherein:
V is an optional cleavable moiety,
(W)ₖ—(X)₁-A is an elongated, self-eliminating spacer system, that self-eliminates via a 1,(4+2n)-elimination,
W and X are each a 1,(4+2n) electronic cascade spacer, being the same or different,
A is either a spacer group of formula (Y)ₘ, wherein Y is a 1,(4+2n) electronic cascade spacer, or a group of formula U, being a cyclisation elimination spacer,
k, l and m are independently an integer of 0 (included) to 5 (included),
n is an integer of 0 (included) to 10 (included),
   with the provisos that:
when A is (Y)ₘ: then k+l+m≥1, and
if k+l+m=l, then n>l;
when A is U: then k+1≥1.
   W, X, and Y are independently selected from compounds having the formula:

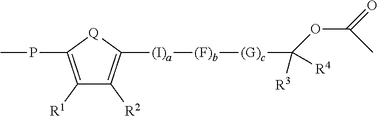

or the formula:

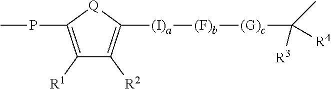

wherein: Q is —R⁵C=CR⁶—, S, O, NR⁵, —R⁵C═N—, or —N═CR⁵—
   P is NR⁷, O or S
   a, b, and c are independently an integer of 0 (included) to 5 (included);
   I, F and G are independently selected from compounds having the formula:

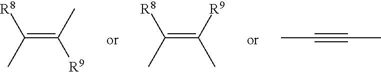

wherein R¹, R², R³, R⁴, R⁵, R⁶, R⁷, R⁸, and R⁹ independently represent H, C₁₋₆ alkyl, C₃₋₂₀ heterocyclyl, C₅₋₂₀ aryl, C₁₋₆ alkoxy, hydroxy (OH), amino (NH₂), mono-substituted amino (NRₓH), di-substituted amino (NRₓ¹Rₓ²), nitro (NO₂), halogen, CF₃, CN, CONH₂, SO₂Me, CONHMe, cyclic C₁₋₅ alkylamino, imidazolyl, C₁₋₆ alkylpiperazinyl, morpholino, thiol (SH), thioether (SRₓ), tetrazole, carboxy (COOH), carboxylate (COORₓ), sulphoxy (S(═O)₂OH), sulphonate (S(═O)₂ORₓ), sulphonyl (S(═O)₂Rₓ), sulphixy (S(═O)OH), sulphinate (S(═O)ORₓ), sulphinyl (S(═O)Rₓ), phosphonooxy (OP(═O)(OH)₂), and phosphate (OP(═O)(ORₓ)₂), where Rₓ, Rₓ¹ and Rₓ² are independently selected from a C₁₋₆ alkyl group, a C₃₋₂₀ heterocyclyl group or a C₅₋₂₀ aryl group, two or more of the substituents R¹, R², R³, R⁴, R⁵, R⁶, R⁷, R⁸, or R⁹ optionally being connected to one another to form one or more aliphatic or aromatic cyclic structures;
   U is selected from compounds having the formula:

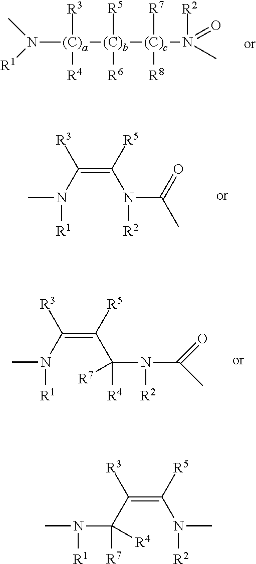

wherein:
   a, b and c are independently selected to be an integer of 0 or 1; provided that a+b+c=2 or 3;
   R¹ and/or R² independently represent H, C₁₋₆ alkyl, said alkyl being optionally substituted with one or more of the following groups: hydroxy (OH), ether (ORₓ), amino (NH₂), mono-substituted amino (NRₓH), disubstituted amino (NRₓ¹Rₓ²), nitro (NO₂), halogen, CF₃, CN, CONH₂, SO₂Me, CONHMe, cyclic C₁₋₅ alkylamino, imidazolyl, C₁₋₆ alkylpiperazinyl, morpholino, thiol (SH), thioether (SRₓ), tetrazole, carboxy (COOH), carboxylate (COORₓ), sulphoxy (S(═O)₂OH), sulphonate (S(═O)₂₀Rₓ), sulphonyl (S(═O)₂Rₓ), sulphixy (S(═O)OH), sulphinate (S(═O)ORₓ), sulphinyl (S(═O)Rₓ), phosphonooxy (OP(═O)(OH)₂), and phosphate (OP(═O)(ORₓ)₂), where Rₓ, Rₓ¹ and Rₓ² are selected from a C₁₋₆ alkyl group, a C₃₋₂₀ heterocyclyl group or a C₅₋₂₀ aryl group; and
   R³, R⁴, R⁵, R⁶, R⁷ and R⁸ independently represent H, C₁₋₆ alkyl, C₃₋₂₀ heterocyclyl, C₅₋₂₀ aryl, C₁₋₆ alkoxy, hydroxy (OH), amino (NH₂), mono-substituted amino (NRₓH), disubstituted amino (NRₓ¹Rₓ²), nitro (NO₂), halogen, CF₃, CN, CONH₂, SO₂Me, CONHMe, cyclic C₁₋₅ alkylamino, imidazolyl, C₁₋₆ alkylpiperazinyl, morpholino, thiol (SH), thioether (SRₓ), tetrazole, carboxy (COOH), carboxylate (COORₓ), sulphoxy (S(═O)₂OH), sulphonate (S(═O)₂ORₓ), sulphonyl (S(═O)₂Rₓ), sulphixy (S(═O)OH), sulphinate (S(═O)ORₓ), sulphinyl (S(═O)Rₓ), phosphonooxy (OP(═O)(OH)₂), and phosphate (OP(═O)(ORₓ)₂), where Rₓ, Rₓ¹ and Rₓ² are selected from a C₁₋₆ alkyl group, a C₃₋₂₀ heterocyclyl group or a C₅₋₂₀ aryl group, and two or more of the substituents R¹, R², R³, R⁴, R⁵, R⁶, R⁷, or R⁸ are optionally connected to one another to form one or more aliphatic or aromatic cyclic structures.

In embodiments, LM Linker molecules can include the following moieties:
p-aminobenzyloxy carbonyl-p-aminobenzyloxy carbonyl;
p-aminobenzyloxy carbonyl-p-aminobenzyloxy carbonyl-p-aminobenzyloxycarbonyl;
p-ammocinnamyloxy carbonyl;
p-aminocinnamyloxycarbonyl-p-aminobenzyloxy carbonyl;
p-amino-benzyloxy carbonyl-p-aminocinnamyloxycarbonyl;
p-aminocinnamyloxy carbonyl-p-aminocinnamyloxycarbonyl;
p-aminophenylpentadienyloxy carbonyl;
p-aminophenylpentadienyloxycarbonyl-p-aminocinnamyloxycarbonyl;
p-aminophenylpentadienyloxycarbonyl-p-aminobenzyloxy carbonyl;
p-aminophenylpentadienyloxy carbonyl-p-aminophenylpentadienyloxy carbonyl;
p-aminobenzyloxycarbonyl(methylamino)ethyl(methylamino)carbonyl;
p-aminocinnamyloxycarbonyl(methylamino)ethyl(methylamino)carbonyl;
p-aminobenzyloxy carbonyl-p-aminobenzyloxycarbonyl(methylamino)ethyl(methylamino)carbonyl;
p-aminocinnamyloxy carbonyl-p-aminobenzyloxycarbonyl(methylamino)ethyl(methylamino)carbonyl;
p-aminobenzyloxy carbonyl-p-aminocinnamyloxycarbonyl(methylamino)ethyl(methylamino)-carbonyl;
p-aminocinnamyloxy carbonyl-p-aminocinnamyloxycarbonyl(methylamino)ethyl(methylamino)carbonyl;
p-aminobenzyloxy carbonyl-p-aminobenzyl;
p-aminobenzyloxy carbonyl-p-aminobenzyloxy carbonyl-p-aminobenzyl;
p-aminocinnamyl;
p-aminocinnamyloxy carbonyl-p-aminobenzyl;
p-aminobenzyloxy carbonyl-p-aminocinnamyl;
p-amino-cinnamyloxy carbonyl-p-aminocinnamyl;
p-aminophenylpentadienyl;
p-aminophenylpentadienyloxycarbonyl-p-aminocinnamyl;
p-aminophenylpentadienyloxycarbonyl-p-aminobenzyl; and
p-aminophenylpentadienyloxycarbonyl-p-aminophenylpentadienyl.

In some embodiments, a B7-H3-ADC comprises two, three, four, five, six, seven, eight, nine or ten cytotoxic duocarmycin moieties, which may be the same, or may independently be the same or different from another cytotoxic duocarmycin moiety of the B7-H3-ADC. In one embodiment, each such cytotoxic duocarmycin moiety is conjugated to the Ab of a B7-H3-ADC via a separate Linker Molecule. Alternatively, more than one cytotoxic duocarmycin moiety may be attached to the Ab of a B7-H3-ADC via the same Linker Molecule.

Cytotoxic duocarmycin moieties may be conjugated to the Ab of a B7-H3-ADC by means known in the art (see, e.g., Yao, H. et al. (2016) “Methods to Design and Synthesize Antibody-Drug Conjugates (ADC),” Intl. J. Molec. Sci. 17(194):1-16); Behrens, C. R. et al. (2014) “Methods For Site-Specific Drug Conjugation To Antibodies,” mAbs 6(1):46-53; Bouchard, H. et al. (2014) “Antibody-Drug Conjugates—A New Wave Of Cancer Drugs,” Bioorganic & Medicinal Chem. Lett 24:5357-5363). The thiol group of a cysteine, the amino side group of lysine, glutamine or arginine, or the carboxyl group of glutamate or aspartate can be employed to conjugate the Linker Molecule-cytotoxic duocarmycin moiety (LM-D) to the Ab of a B7-H3-ADC. Native antibodies contain numerous lysine conjugation sites, and thus are capable of linking multiple conjugated molecules per antibody. Indeed, peptide mapping has determined that conjugation occurs on both the heavy and light chain at approximately 20 different lysine residues (40 lysines per mAb). Therefore, greater than one million different ADC species can be generated. Cysteine conjugation occurs after reduction of one to four inter-chain disulfide bonds, and the conjugation is thus limited in native VL and VH Domains to the eight exposed sulfhydryl groups. However, if desired, additional reactive (e.g., lysine, cysteine, selenocysteine, etc.) residues may be engineered into an antibody (e.g., within a VL Domain and/or a VH Domain and/or a Constant Domain). For example, one or more native amino acid residues may be substituted with a cysteine residue. An unnatural amino acid (e.g. p-acetylphenylalanine) may be genetically incorporated into an antibody using an amber stop codon suppressor tRNA/aaRS pair. (See, e.g., Behrens CR, and Liu B. (2014) “Methods For Site-Specific Drug Conjugation To Antibodies,” mAbs 6(1):46-53. doi:10.4161/mabs.26632; Panowksi, S., et al. (2014) “Site-Specific Antibody Drug Conjugates For Cancer Therapy,” mAbs, 6(1), 34-45, doi:10.4161/mabs.27022; and WO 2008/070593). Alternatively, or additionally, enzymes (e.g., a glycotransferase) may be used to conjugate the Linker Molecule-cytotoxic duocarmycin moiety (LM-D) to the Ab of a B7-H3-ADC. The glycotransferase platform attaches a sugar moiety to a glycosylation site on an antibody (for example, position N297 of the Fc Domain of a human IgG antibody), which can then serve as the Linker Molecule and conjugate the cytotoxic duocarmycin moiety (D) to the Ab of a B7-H3-ADC. Alternatively, a transglutaminase may be used to catalyze the formation of a covalent bond between a free amine group and a glutamine side chain.

A representative transglutaminase is the commercially available transglutaminase from Streptoverticillium mobaraense (mTG) (Pastemack, R. et al. (1998) “Bacterial Pro-Transglutaminase From Streptoverticillium mobaraense—Purification, Characterisation And Sequence Of The Zymogen,” Eur. J. Biochem. 257(3):570-576; Yokoyama, K. et al. (2004) “Properties And Applications Of Microbial Transglutaminase,” Appl. Microbiol. Biotechnol. 64:447-454). This enzyme does not recognize any of the natural occurring glutamine residues in the Fc Domain of glycosylated antibodies, but does recognize the tetrapeptide LLQL (SEQ ID NO:21) (Jeger, S. et al. (2010) “Site-Specific And Stoichiometric Modification Of Antibodies By Bacterial Transglutaminase,” Angew Chem. Int. Ed. Engl. 49:9995-9997) that may be engineered into a VL Domain and/or a VH Domain and/or a Constant Domain. Such considerations are reviewed by Panowski, S. et al. (2014) “Site-Specific Antibody Drug Conjugates For Cancer Therapy,” mAbs 6(1):34-45.

### B. Duocarmycin Moieties

Duocarmycins are members of a series of related natural products first isolated from Streptomyces bacteria and they are potent antitumor antibiotics (see Dokter, W. et al. (2014) “Preclinical Profile of the HER2-Targeting ADC SYD983/SYD985: Introduction of a New Duocarmycin-Based Linker-Drug Platform,” Mol. Cancer Ther. 13(11):2618-2629; Boger, D. L. et al. (1991). “Duocarmycins—A New Class Of Sequence Selective DNA Minor Groove Alkylating Agents,” Chemtracts: Organic Chemistry 4 (5): 329-349 (1991); Tercel et al. (2013) “The Cytotoxicity Of Duocarmycin Analogues Is Mediated Through Alkylation Of DNA, Not Aldehyde Dehydrogenase 1: A Comment,” Chem. Int. Ed. Engl. 52(21):5442-5446; Boger, D. L. et al. (1995) “CC-1065 And The Duocarmycins: Unraveling The Keys To A New Class Of Naturally Derived DNA Alkylating Agents,” Proc. Natl. Acad. Sci. (U.S.A.) 92(9):3642-3649; Cacciari, B. et al. (2000) “CC-1065 And The Duocarmycins: Recent Developments,” Expert Opinion on Therapeutic Patents 10(12):1853-1871).

Natural duocarmycins include duocarmycin A, duocarmycin B1, duocarmycin B2, duocarmycin C1, duocarmycin C2, duocarmycin D, duocarmycin SA, and CC-1065 (PCT Publication No. WO 2010/062171; Martin, D. G. et al. (1980) “Structure Of CC-1065 (NSC 298223), A New Antitumor Antibiotic,” J. Antibiotics 33:902-903; Boger, D. L. et al. (1995) “CC-1065 And The Duocarmycins: Unraveling The Keys To A New Class Of Naturally Derived DNA Alkylating Agents,” Proc. Natl. Acad. Sci. (U.S.A.) 92:3642-3649).

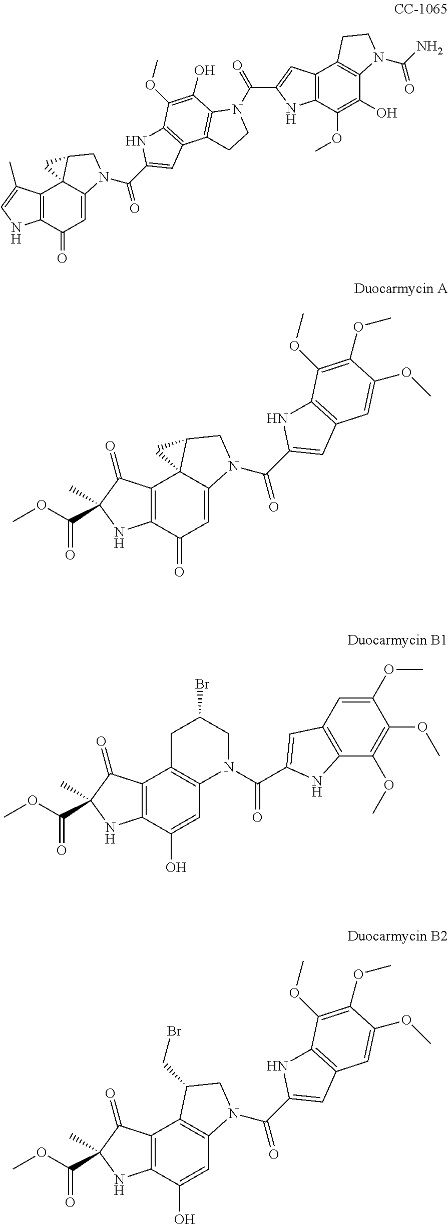

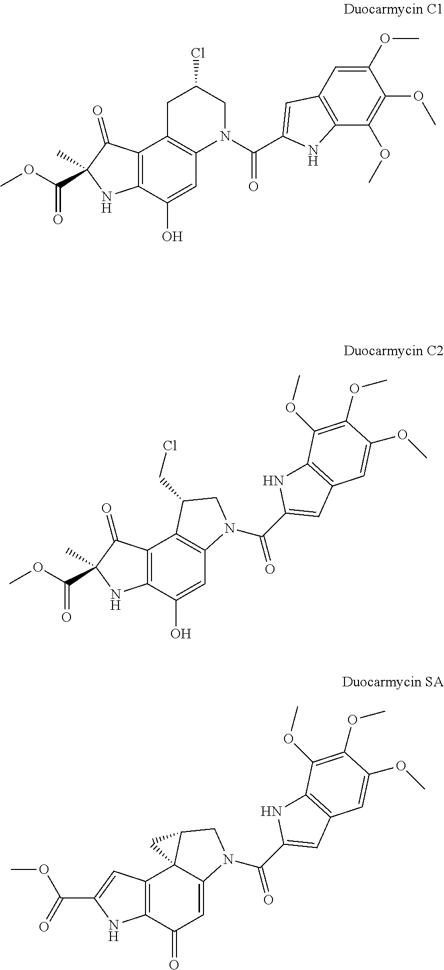

Suitable synthetic duocarmycin analogs include adozelesin, bizelesin, carzelesin (U-80244) and spiro-duocarmycin (spiro-DUBA) (Dokter, W. et al. (2014) “Preclinical Profile of the HER2-Targeting ADC SYD983 SYD985: Introduction of a New Duocarmycin-Based Linker-Drug Platform,” Mol. Cancer Ther. 13(11):2618-2629; Elgersma, R. C. et al. (2014) “Design, Synthesis, and Evaluation of Linker-Duocarmycin Payloads: Toward Selection of HER2-Targeting Antibody—Drug Conjugate SYD985,” Mol. Pharmaceut. 12:1813-1835):

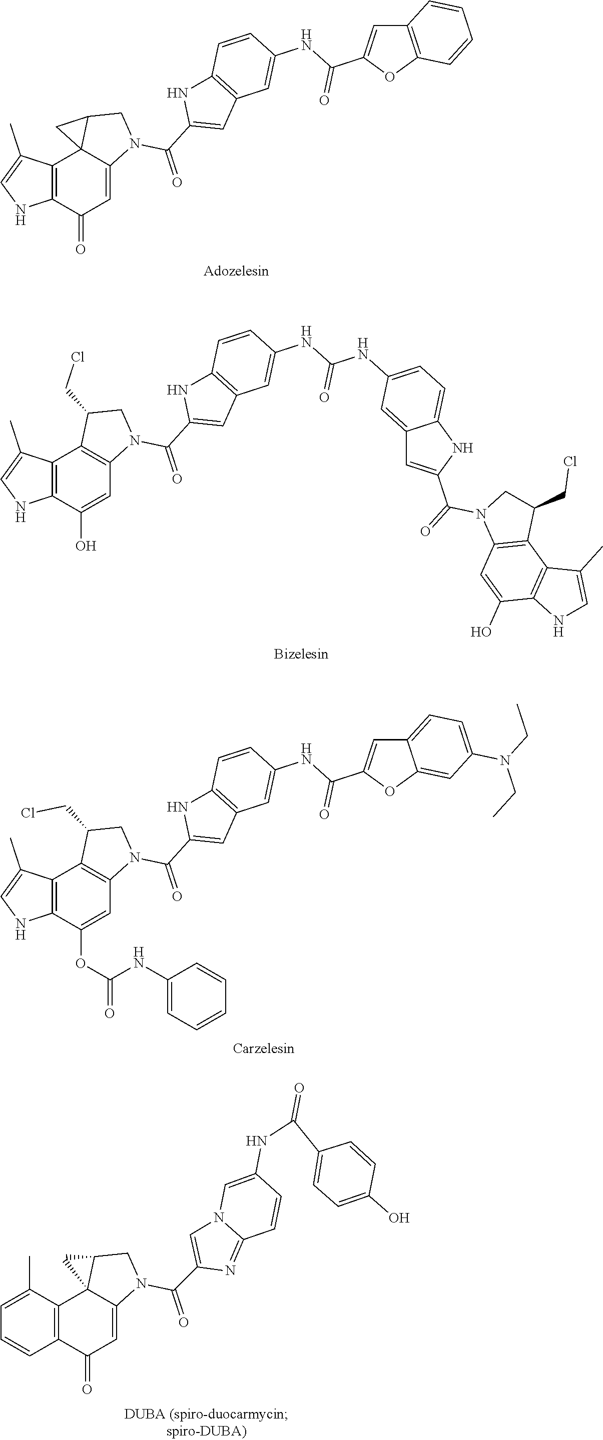 [SMILES: Cc1c[nH]c2c1C13CC1CN(C(=O)c1cc4cc(NC(=O)c5cc6ccccc6o5)ccc4[nH]1)C3=CC2=O ; Cc1c[nH]c2c(O)cc3c(c12)[C@H](CCl)CN3C(=O)c1cc2cc(NC(=O)Nc3ccc4[nH]c(C(=O)N5C[C@@H](CCl)c6c5cc(O)c5[nH]cc(C)c65)cc4c3)ccc2[nH]1 ; CCN(CC)c1ccc2cc(C(=O)Nc3ccc4[nH]c(C(=O)N5C[C@@H](CCl)c6c5cc(OC(=O)Nc5ccccc5)c5[nH]cc(C)c65)cc4c3)oc2c1 ; Cc1cccc2c1[C@@]13C[C@@H]1CN(C(=O)c1cn4cc(NC(=O)c5ccc(O)cc5)ccc4n1)C3=CC2=O]

Additional synthetic duocarmycin analogs include those disclosed in PCT Publication No. WO 2010/062171, and particularly such analogs that have the formula:

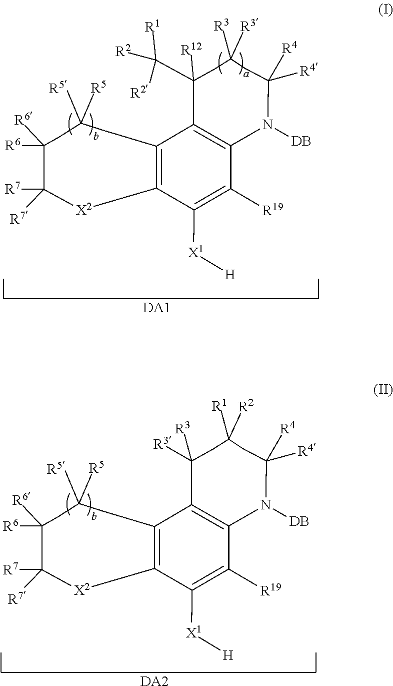

or a pharmaceutically acceptable salt, hydrate, or solvate thereof, wherein DB is a DNA-binding moiety and is selected from the group consisting of:

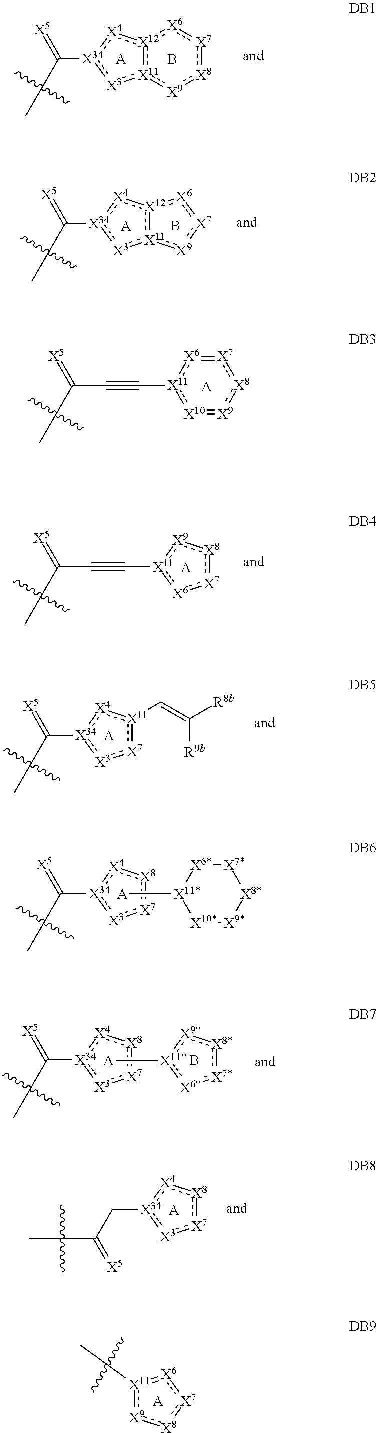

wherein:
R is a leaving group;
R², R^{2′}, R³, R^{3′}, R⁴, R^{4′}, R¹², and R¹⁹ are independently selected from H, OH, SH, NH₂, N₃, NO₂, NO, CF₃, CN, C(O)NH₂, C(O)H, C(O)OH, halogen, Ra, SR^{a}, S(O)R^{a}, S(O)₂R^{a}, S(O)OR^{a}, S(O)₂OR^{a}, OS(O)R^{a}, OS(O)₂R^{a}, OS(O)OR^{a}, OS(O)₂OR^{a}, OR^{a}, NHR^{a}, N(R^{a})R^{b}, +N(R^{a})(R^{b})R^{c}, P(O)(OR^{a})(OR^{b}), OP(O)(OR^{a})(OR^{b}), SiR^{a}R^{b}R^{c}, C(O)R^{a}, C(O)OR^{a}, C(O)N(R^{a})R^{b}, OC(O)R^{a}, OC(O)OR^{a}, OC(O)N(R^{a})R^{b}, N(R^{a})C(O)R^{b}, N(R^{a})C(O)OR^{b}, and N(R^{a})C(O)N(R^{b})R^{c}, wherein R^{a}, R^{b}, and R^{c} are independently selected from H and optionally substituted C₁₋₃ alkyl or C₁₋₃ heteroalkyl, or R³+R^{3′} and/or R⁴+R^{4′} are independently selected from ═O, ═S, ═NOR¹⁸, =C(R¹⁸)R^{18′}, and =NR¹⁸, R¹⁸ and R^{18′} being independently selected from H and optionally substituted C₁₋₃ alkyl, two or more of R², R^{2′}, R³, R^{3′}, R⁴, R^{4′} and R¹² optionally being joined by one or more bonds to form one or more optionally substituted carbocycles and/or heterocycles;
X² is selected from O, C(R¹⁴)(R^{14′}), and NR^{14′}, wherein R¹⁴ and R^{14′} have the same meaning as defined for R⁷ and are independently selected, or R^{14′} and R^{7′} are absent resulting in a double bond between the atoms designated to bear R^{7′} and R^{14′};
R⁵, R^{5′}, R⁶, R^{6′}, R⁷, and R^{7′} are independently selected from H, OH, SH, NH₂, N₃, NO₂, NO, CF₃, CN, C(O)NH₂, C(O)H, C(O)OH, halogen, R^{e}, SR^{e}, S(O)R^{e}, S(O)₂R^{e}, S(O)OR^{e}, S(O)₂OR^{e}, OS(O)R^{e}, OS(O)₂R^{e}, OS(O)OR^{e}, OS(O)₂OR^{e}, OR^{e}, NHR^{e}, N(R^{e})R^{f}, +N(R^{e})(R^{f})R^{g}, P(O)(OR^{e})(OR^{f}), OP(O)(OR^{e})(OR^{f}), SiR^{e}R^{f}R^{g}, C(O)R^{g}, C(O)OR^{e}, C(O)N(R^{e})R^{f}, OC(O)R^{e}, OC(O)OR^{e}, OC(O)N(R^{e})R^{f}, N(R^{e})C(O)R^{f}, N(R^{e})C(O)OR^{f}, N(R^{e})C(O)N(R^{f})R^{g}, and a water-soluble group,
   wherein
R^{e}, R^{f}, and R^{g} are independently selected from H and optionally substituted (CH₂CH₂O)ₑₑCH₂CH₂X¹³R^{e1}, C₁₋₁₅ alkyl, C₁₋₁₅ heteroalkyl, C₃₋₁₅ cycloalkyl, C₁₋₁₅ heterocycloalkyl, C₅₋₁₅ aryl, or C₁₋₁₅ heteroaryl, wherein ee is selected from 1 to 1000, X¹³ is selected from O, S, and NR^{f1}, and R^{f1} and R^{e1} are independently selected from H and C₁₋₃ alkyl, one or more of the optional substituents in R^{e}, R^{f}, and/or R^{g} optionally being a water-soluble group, two or more of R^{e}, R^{f}, and R^{g} optionally being joined by one or more bonds to form one or more optionally substituted carbocycles and/or heterocycles, or R⁵+R^{5′} and/or R⁶+R^{6′} and/or R⁷+R^{7′} are independently selected from ═O, ═S, ═NOR^{e3}, =C(R^{e3})R^{e4}, and =NR^{e3}, R^{e3} and R^{e4} being independently selected from H and optionally substituted C₁₋₃ alkyl, or R^{5′}+R^{6′} and/or R^{6′}+R^{7′} and/or R^{7′}+R^{14′} are absent, resulting in a double bond between the atoms designated to bear R^{5′}+R^{6′} and/or R^{6′}+R^{7′} and/or R^{7′}+R^{14′} respectively, two or more of R⁵, R^{5′}, R⁶, R^{6′}, R⁷, R^{7′}, R¹⁴ and R^{14′} optionally being joined by one or more bonds to form one or more optionally substituted carbocycles and/or heterocycles;
X¹ is selected from O, S, and NR, wherein R is selected from H and optionally substituted C₁₋₈ alkyl or C₁₋₈ heteroalkyl and not joined with any other substituent;
X³ is selected from O, S, C(R¹⁵)R^{15′}, —C(R¹⁵)(R^{15′})—C(R^{15″})(R^{15″})—, —N(R¹⁵)—N(R^{15′})—, —C(R¹⁵)(R^{15′})—N(R^{15′})—, —N(R^{15″})—C(R¹⁵)(R^{15′})—, —C(R¹⁵)(R^{15′})—O—, —O—C(R¹⁵)(R^{15′})—, —C(R¹⁵)(R^{15′})—S—, —S—C(R¹⁵)(R^{15′})—, —C(R¹⁵)═C(R^{15′})—, =C(R¹⁵)—C(R^{15′})=, —N═C(R¹⁵)—, =N—C(R^{15′})=, —C(R¹⁵)=N—, =C(R¹⁵)—N=, —N═N—, =N—N=, CR¹⁵, N, NR¹⁵, or in DB1 and DB2-X3- represents —X^{3a} and X^{3b}—, wherein X^{3a} is connected to X³⁴, a double bond is present between X³⁴ and X⁴, and X^{3b} is connected to X^{3a}, wherein X^{3a} is independently selected from H and optionally substituted (CH₂CH₂O)ₑₑCH₂CH₂X³R^{e1}, C₁₋₈ alkyl, or C₁₋₈ heteroalkyl and not joined with any other substituent;
X⁴ is selected from O, S, C(R¹⁶)R^{16′}, NR¹⁶, N, and CR¹⁶;
X⁵ is selected from O, S, C(R¹⁷)R¹⁷, NOR¹⁷, and NR¹⁷, wherein R¹⁷ and R^{17′} are independently selected from H and optionally substituted C₁₋₈ alkyl or C₁₋₈ heteroalkyl and not joined with any other substituent;
X⁶ is selected from CR¹¹, CR¹¹(R^{11′}), N, NR¹¹, O, and S;
X⁷ is selected from CR⁸, CR⁸(R^{8′}), N, NR⁸, O, and S;
X⁸ is selected from CR⁹, CR⁹(R^{9′}), N, NR⁹, O, and S;
X⁹ is selected from CR¹⁰, CR¹⁰(R^{10′}), N, NR¹⁰, O, and S;
X¹⁰ is selected from CR²⁰, CR²⁰(R^{20′}), N, NR²⁰, O, and S;
X¹¹ is selected from C, CR²¹, and N, or X¹¹—X^{3b} is selected from CR²¹, CR²¹(R²¹), N, NR²¹, O, and S;
X¹² is selected from C, CR²², and N;
X^{6*}, X^{7*}, X^{8*}, X^{9*}, X^{10*}, and X^{11*} have the same meaning as defined for X⁶, X⁷, X⁸, X⁹, X¹⁰, and X¹¹, respectively, and are independently selected;
X³⁴ is selected from C, CR²³, and N;
the ring B atom of X^{11*} in DB6 and DB7 is connected to a ring atom of ring A such that ring A and ring B in DB6 and DB7 are directly connected via a single bond; a dashed double bond means that the indicated bond may be a single bond or a non-cumulated, optionally delocalized, double bond;
R⁸, R^{8′}, R⁹, R^{9′}, R¹⁰, R^{10′}, R¹¹, R^{11′}, R¹⁵, R^{15′}, R^{15″}, R¹⁶, R^{16′}, R²⁰, R^{20′}, R²¹, R^{21′}, R²², and R²³ are each independently selected from H, OH, SH, NH₂, N₃, NO₂, NO, CF₃, CN, C(O)NH₂, C(O)H, C(O)OH, halogen, R^{h}, SR^{h}, S(O)R^{h}, S(O)₂R^{h}, S(O)OR^{h}, S(O)₂OR^{h}, OS(O)R^{h}, OS(O)₂R^{h}, OS(O)OR^{h}, OS(O)₂OR^{h}, OR^{h}, NHR^{h}, N(R^{h})Rⁱ, +N(R^{h})(Rⁱ)R^{j}, P(O)(OR^{h})(ORⁱ), OP(O)(OR^{h})(ORⁱ), SiR^{h}RⁱR^{j}, C(O)R^{h}, C(O)OR^{h}, C(O)N(R^{h})Rⁱ, OC(O)R^{h}, OC(O)OR^{h}, OC(O)N(R^{h})Rⁱ, N(R^{h})C(O)Rⁱ, N(R^{h})C(O)ORⁱ, N(R^{h})C(O)N(Rⁱ)R^{j}, and a water-soluble group, wherein
R^{h}, Rⁱ, and R^{j} are independently selected from H and optionally substituted (CH₂CH₂O)ₑₑCH₂CH₂X¹³R^{e}, C₁₋₁₅ alkyl, C₁₋₁₅ heteroalkyl, C₃₋₁₅ cycloalkyl, C₁₋₁₅ heterocycloalkyl, C₅₋₁₅ aryl, or C₁₋₁₅ heteroaryl, one or more of the optional substituents in R^{h}, Rⁱ, and/or R^{j} optionally being a water-soluble group, two or more of R^{h}, Rⁱ, and R^{j} optionally being joined by one or more bonds to form one or more optionally substituted carbocycles and/or heterocycles,
or R⁸+R^{8′} and/or R⁹+R^{9′} and/or R¹⁰+R^{10′} and/or R¹¹+R^{11′} and/or R¹⁵+R^{15′} and/or R^{15″}+R^{15′} and/or R¹⁶+R^{16′} and/or R²⁰+R^{20′} and/or R²¹+R^{21′} are independently selected from ═O, ═S, ═NOR^{h1}, =C(R^{h1})R^{h2}, and =NR^{h1}, R^{h1} and R^{h2} being independently selected from H and optionally substituted C₁₋₃ alkyl, two or more of R⁸, R^{8′}, R⁹, R^{9′}, R¹⁰, R^{10′}, R¹¹, R^{11′}, R¹⁵, R^{15′}, R^{15″}, R^{15′″}, R¹⁶, R²⁰, R^{20′}, R²¹, R^{21′}, R²², and R²³ optionally being joined by one or more bonds to form one or more optionally substituted carbocycles and/or heterocycles;
R^{8b} and R^{9b} are independently selected and have the same meaning as R⁸, except that they may not be joined with any other substituent;
one of R⁴ and R^{4′} and one of R¹⁶ and R^{16′} may optionally be joined by one or more bonds to form one or more optionally substituted carbocycles and/or heterocycles;
one of R⁴ and R^{4′} and one of R¹⁶ and R^{16′} may optionally be joined by one of R², R^{2′}, R³, and R^{3′} and one of R⁵ and R^{5′} may optionally be joined by one or more bonds to form one or more optionally substituted carbocycles and/or heterocycles; a and b are independently selected from 0 and 1;
the DB moiety does not comprise a DAI, DA2, DAI′, or DA2′ moiety; ring B in DB1 is a heterocycle;
if X³ in DB1 represents —X^{3a} and X^{3b}— and ring B is aromatic, then two vicinal substituents on said ring B are joined to form an optionally substituted carbocycle or heterocycle fused to said ring B;
if X³ in DB2 represents —X^{3a} and X^{3b}— and ring B is aromatic, then two vicinal substituents on said ring B are joined to form an optionally substituted heterocycle fused to said ring B, an optionally substituted non-aromatic carbocycle fused to said ring B, or a substituted aromatic carbocycle which is fused to said ring B and to which at least one substituent is attached that contains a hydroxy group, a primary amino group, or a secondary amino group, the primary or secondary amine not being a ring atom in an aromatic ring system nor being part of an amide;
if ring A in DB2 is a 6-membered aromatic ring, then substituents on ring B are not joined to form a ring fused to ring B;
two vicinal substituents on ring A in DB8 are joined to form an optionally substituted carbocycle or heterocycle fused to said ring A to form a bicyclic moiety to which no further rings are fused; and ring A in DB9 together with any rings fused to said ring A contains at least two ring heteroatoms.

The above-described Linker Molecules can be conjugated to a cysteine thiol group using thiol-maleimide chemistry, as shown above. In some embodiments, the cytotoxic duocarmycin moiety is a prodrug. For example, the prodrug, vc-seco-DUBA can be conjugated to a self-elimination moiety linked to maleimide linker moiety via a cleavable peptide moiety:

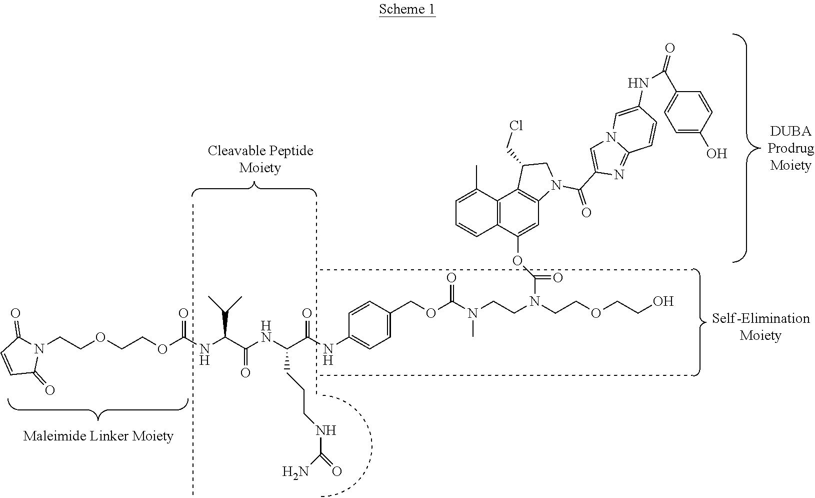 [SMILES: Cc1cccc2c(OC(=O)N(CCOCCO)CCN(C)C(=O)OCc3ccc(NC(=O)[C@H](CCCNC(N)=O)NC(=O)[C@@H](NC(=O)OCCOCCN4C(=O)C=CC4=O)C(C)C)cc3)cc3c(c12)[C@H](CCl)CN3C(=O)c1cn2cc(NC(=O)c3ccc(O)cc3)ccc2n1]

The maleimide linker moiety of the molecule can be conjugated to a thiol group of a cysteine residue of a VL Domain and/or a VH Domain and/or a Constant Domain of the Ab portion of a B7-H3-ADC. Subsequent proteolytic cleavage of the cleavable peptide moiety is followed by the spontaneous elimination of the self-elimination moiety, leading to the release of seco-duocarmycin (seco-DUBA), which spontaneously rearranges to form the active drug, DUBA:

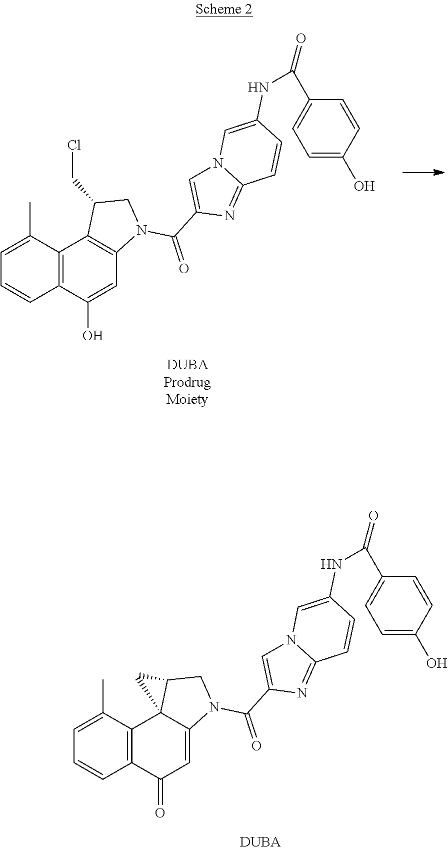 [SMILES: Cc1cccc2c(O)cc3c(c12)[C@H](CCl)CN3C(=O)c1cn2cc(NC(=O)c3ccc(O)cc3)ccc2n1 ; Cc1cccc2c1[C@@]13C[C@@H]1CN(C(=O)c1cn4cc(NC(=O)c5ccc(O)cc5)ccc4n1)C3=CC2=O]

(see, Dokter, W. et al. (2014) “Preclinical Profile of the HER2-Targeting ADC SYD983 SYD985: Introduction of a New Duocarmycin-Based Linker-Drug Platform,” Mol. Cancer Ther. 13(11):2618-2629).

In a non-limiting method for the production of B7-H3-duocarmycin drug moiety conjugates, the method of by Elgersma, R. C. et al. (2014) “Design, Synthesis, and Evaluation of Linker-Duocarmycin Payloads: Toward Selection of HER2-Targeting Antibody-Drug Conjugate SYD985,” Mol. Pharmaceut. 12:1813-1835 or that of WO 2011/133039 will be employed. Thus, a thiol-containing group of the VL or VH chain of an anti-B7-H3 antibody or antibody fragment is conjugated to a seco-DUBA or other prodrug through a Maleimide Linker Moiety-Cleavable Peptide Moiety-Self-Elimination Moiety (Scheme 3A):

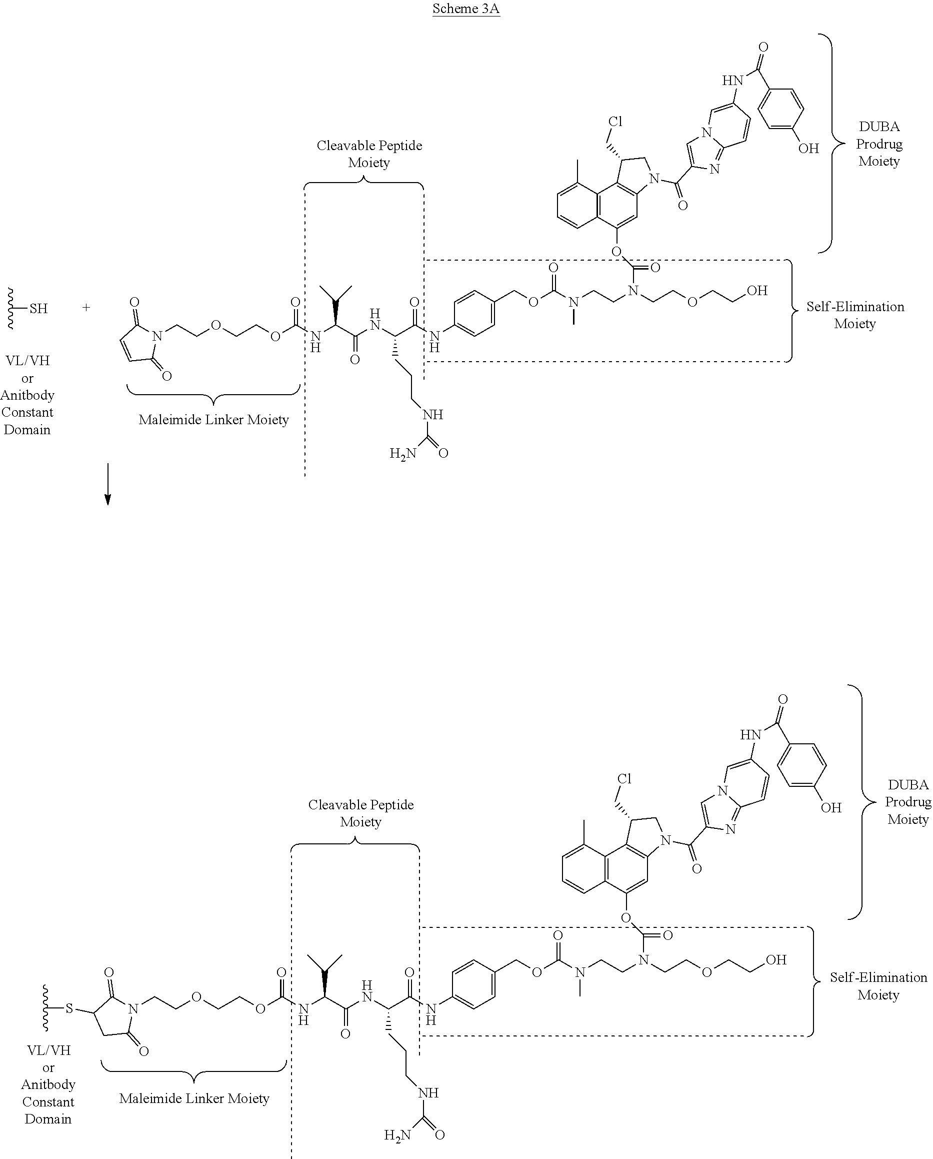

Although the disclosure is illustrated with regard to a DUBA prodrug, other prodrugs, e.g., CC-1065, may be alternatively employed, as shown in Scheme 3B:

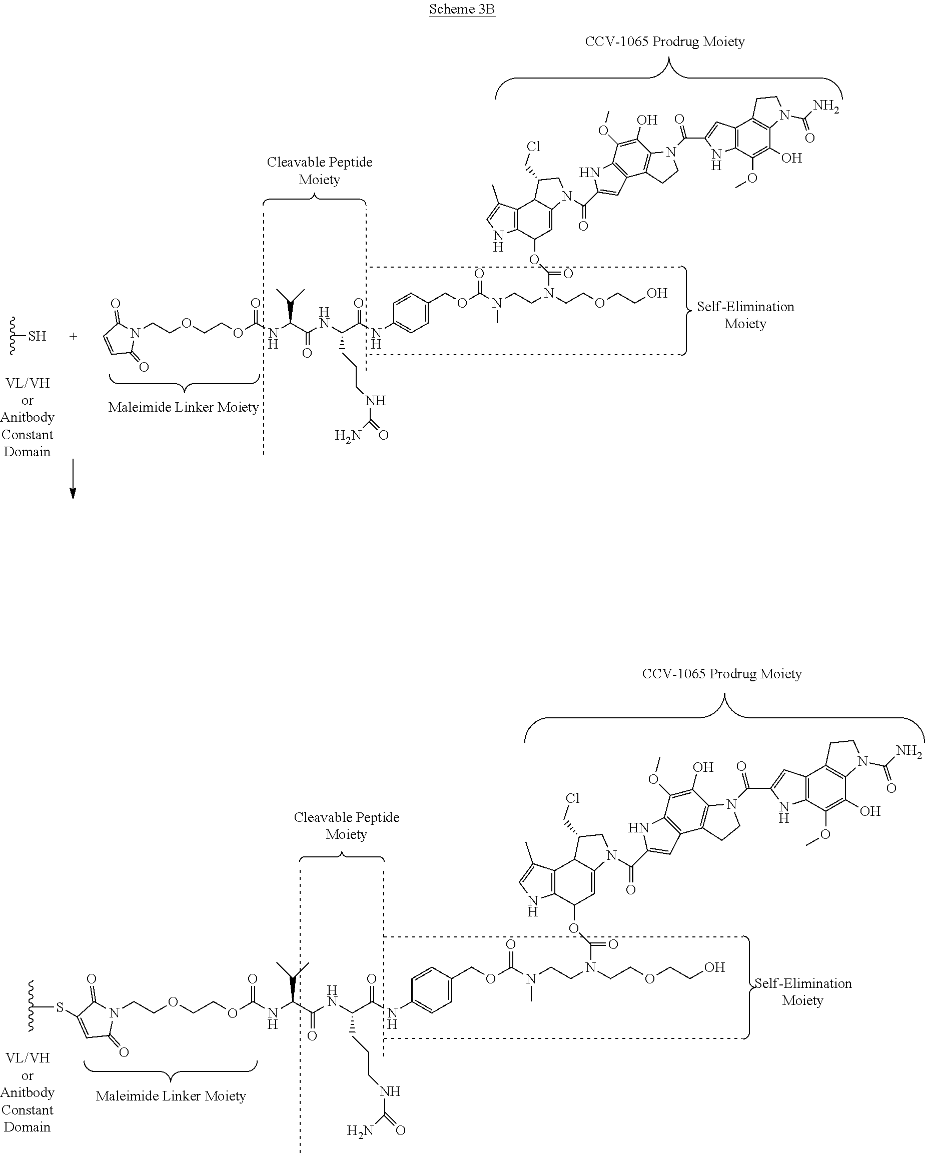

Upon cleavage of the Cleavable Peptide Moiety and elimination of the Self-Elimination Moiety, the Prodrug Moiety is believed to undergo a Winstein spirocyclization to yield the active drug (e.g., DUBA (also referred to herein as spiro-DUBA or spiro-duocarmycin) from seco-DUBA (seco-duocarmycin, i.e., the prodrug) as shown in Scheme 3C).

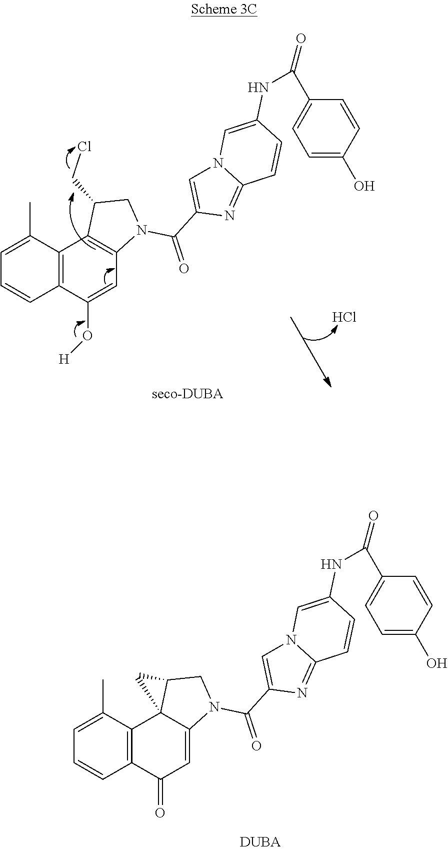 [SMILES: Cc1cccc2c(O)cc3c(c12)[C@H](CCl)CN3C(=O)c1cn2cc(NC(=O)c3ccc(O)cc3)ccc2n1 ; Cl ; Cc1cccc2c1[C@@]13C[C@@H]1CN(C(=O)c1cn4cc(NC(=O)c5ccc(O)cc5)ccc4n1)C3=CC2=O]

seco-DUBA is prepared from the corresponding DNA-alkylating and DNA-binding moieties (e.g., a 1,2,9,9a-tetrahydrocyclopropa-[c]benzo[e]indole-4-one framework as described by Elgersma, R. C. et al. (2014) “Design, Synthesis, and Evaluation of Linker-Duocarmycin Payloads: Toward Selection of HER2-Targeting Antibody—Drug Conjugate SYD985,” Mol. Pharmaceut. 12:1813-1835 (see, Boger, D. L. et al. (1989) “Total Synthesis and Evaluation of (±)-N-(tert-Butoxycarbonyl)-CBI, (±)-CBI-CDPI1, and (±)-CBI-CDPI2: CC-1065 Functional Agents Incorporating the Equivalent 1,2,9,9a-Tetrahydrocyclopropa[1,2-c]benz[1,2-e]indol-4-one (CBI) Left-Hand Subunit,” J. Am. Chem. Soc. 111:6461-6463; Boger, D. L. et al. (1992) “DNA Alkylation Properties of Enhanced Functional Analogs of CC-1065 Incorporating the 1,2,9,9a-Tetrahydrocyclopropa[1,2-c]benz[1,2-e]indol-4-one (CBI) Alkylation Subunit,” J. Am. Chem. Soc. 114:5487-5496).

Scheme 3D illustrates the disclosure by showing the synthesis of the DNA-alkylating moiety for DUBA. Thus, o-tolualdehyde (1) and dimethyl succinate (2) are reacted to produce a mixture of acids (3a/3b) through a Stobbe condensation. Ring closure of the mixture of acids may be accomplished with trifluoroacetic anhydride and gave alcohol (4), which is then protected with benzyl chloride to afford benzyl ether (5). The ensuing hydrolysis of the methyl ester group yields the carboxylic acid (6) which is followed by a Curtius rearrangement in a mixture of toluene and tert-butyl alcohol to provide the carbamate (7). Bromination with N-bromosuccinimide give the bromide (8). The bromide (8) is alkylated with (S)-glycidyl nosylate in the presence of potassium tert-butoxide to give epoxide (9). Reaction with n-butyllithium provides a mixture of desired compound (10) and debrominated, rearranged derivative (11). Yields for desired compound (10) are higher when tetrahydrofuran is used as the solvent and the reaction temperature is kept between −25 and −20° C. Under these conditions, desired compound (10) and debrominated, rearranged derivative (11) are obtainable in an approximate 1:1 ratio. Workup with p-toluenesulfonic acid results in conversion of debrominated, rearranged derivative (11) to (7), thereby aiding recovery of desired compound (10). Mesylation of the hydroxyl group in (10) followed by chloride substitution using lithium chloride gives key intermediate (12).

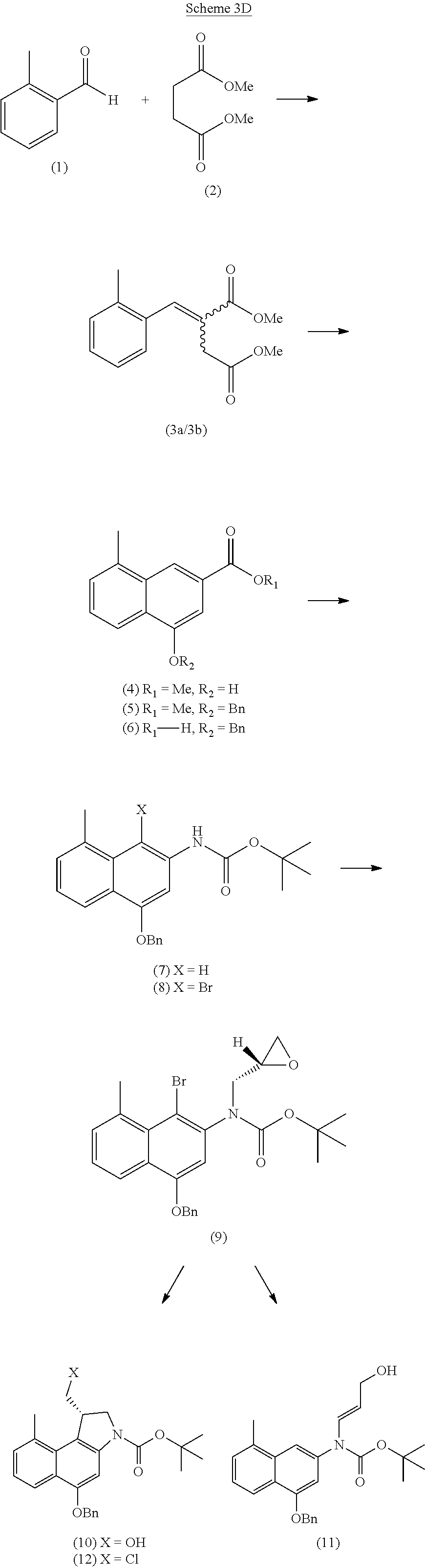

Scheme 3E illustrates the disclosure by showing the synthesis of the DNA-binding moiety for DUBA. Thus, a Chichibabin cyclization reaction is permitted to proceed between ethyl bromopyruvate (13) and 5-nitropyridin-2-amine (14), thereby obtaining nitro compound (15). Reduction of the nitro group with zinc under acidic conditions gives amine (16). Coupling with methoxymethyl (MOM)-protected 4-hydroxybenzoic acid (17), prepared from methyl 4-hydroxybenzoate through reaction with chloromethyl methyl ether followed by ester hydrolysis (see, WO 2004/080979) gives the ethyl ester (18), which may be hydrolyzed with sodium hydroxide in aqueous 1,4-dioxane to provide acid (19).

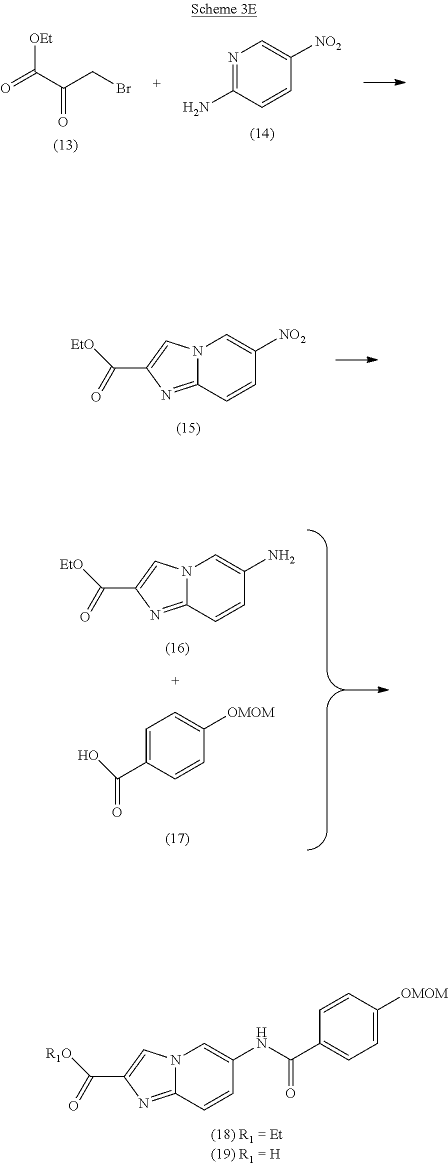

seco-DUBA (also referred to herein as seco-duocarmycin) is then synthesized from DNA-alkylating unit (12) and DNA-binding moiety (19). The tert-butoxycarbonyl (Boc) protective group is removed from (12) under acidic conditions to form the amine (20). EDC-mediated coupling of amine (20) and compound (19) yields protected compound (21), which is then fully deprotected in two consecutive steps (with Pd/C, NH₄HCO2, MeOH/THF, 3 hours, 90%, to yield (22) and then with HCl, 1,4-dioxane/water, 1 h, 95% to provide seco-DUBA (23) as its HCl salt (Scheme 3F).

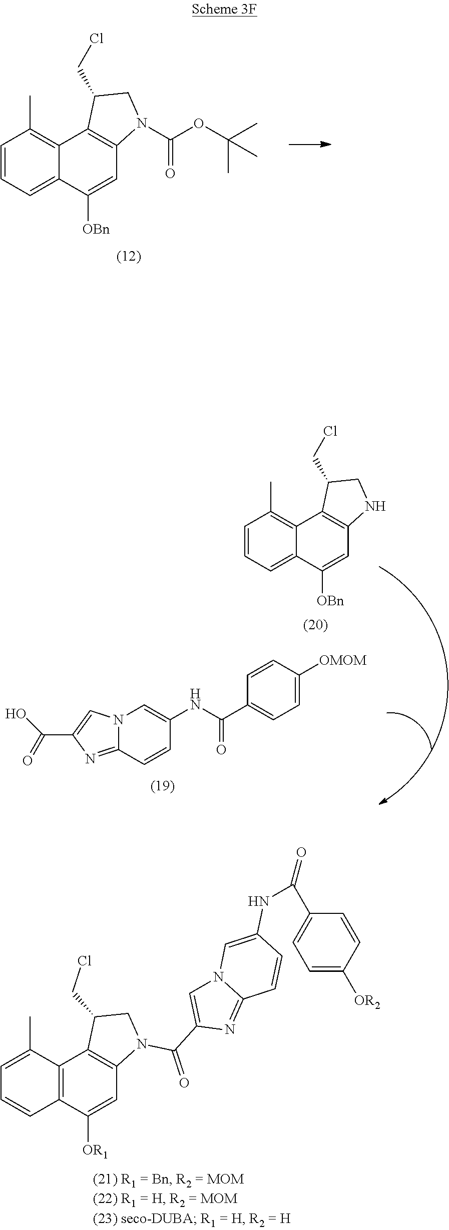

Prodrugs of other drugs, e.g., CC-1065, may be synthesized as described for example in WO 2010/062171.

The Prodrug Moiety may be linked to the other moieties of the ADC according to Scheme 3G. The Maleimide Linker building block was synthesized by starting with a condensation reaction between (24) and 2-(2-aminoethoxy)ethanol (25) to give alcohol (26), which was then converted to reactive carbonate (27) through reaction with 4-nitrophenyl chloroformate. Coupling of (27) to H-Valine-Citrulline-PABA (28), prepared according to Dubowchik, G. M. et al. (2002) “Cathepsin B-Labile Dipeptide Linkers For Lysosomal Release Of Doxorubicin From Internalizing Immunoconjugates: Model Studies Of Enzymatic Drug Release And Antigen-Specific In Vitro Anticancer Activity,” Bioconjugate Chem. 13:855-869) results in the formation of linker (29), which was treated with bis(4-nitrophenyl) carbonate to give activated linker (30).

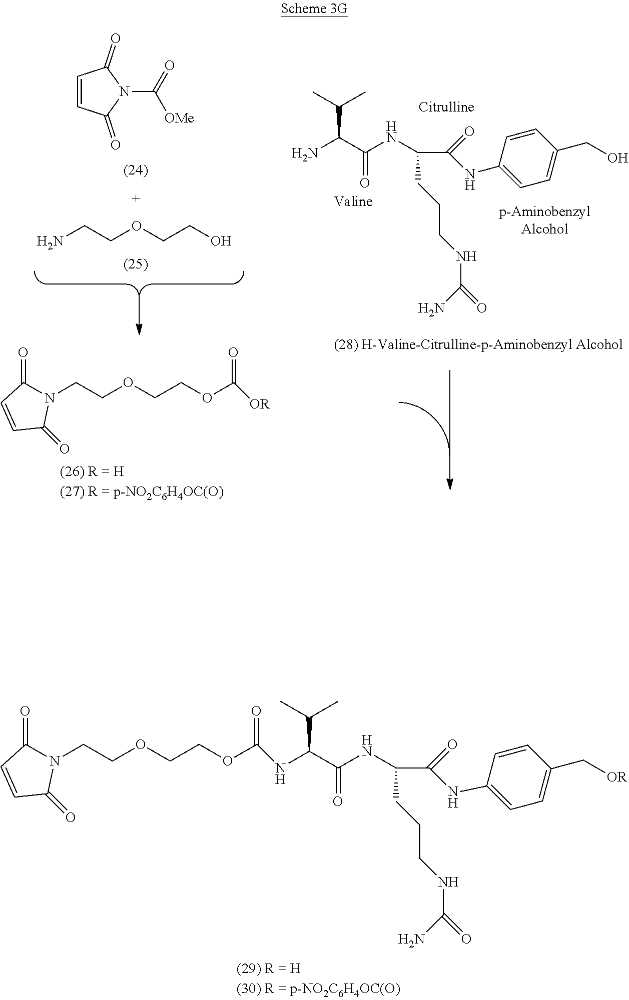

As shown in Scheme 3H, seco-DUBA-MOM (22) is modified for conjugation in two steps. Consecutive treatment of (22) with 4-nitrophenyl chloroformate and tert-butyl methyl(2-(methylamino)ethyl)carbamate (31) gives compound (32). Removal of the Boc and MOM protective groups in (32) with trifluoroacetic acid (TFA) provided (33) as its TFA salt.

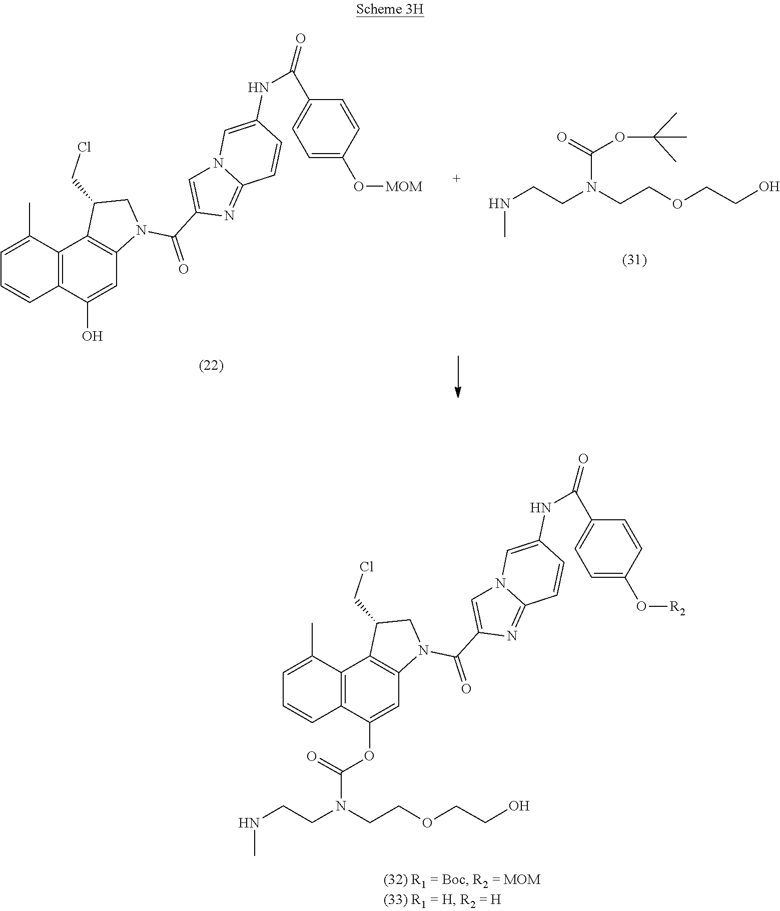

The ADC was synthesized through reaction of activated linker (30) with cyclization spacer-duocarmycin construct (33) under slightly basic conditions. Under these conditions, self-elimination of the cyclization spacer and resulting formation of 3a was suppressed (Scheme 3I).

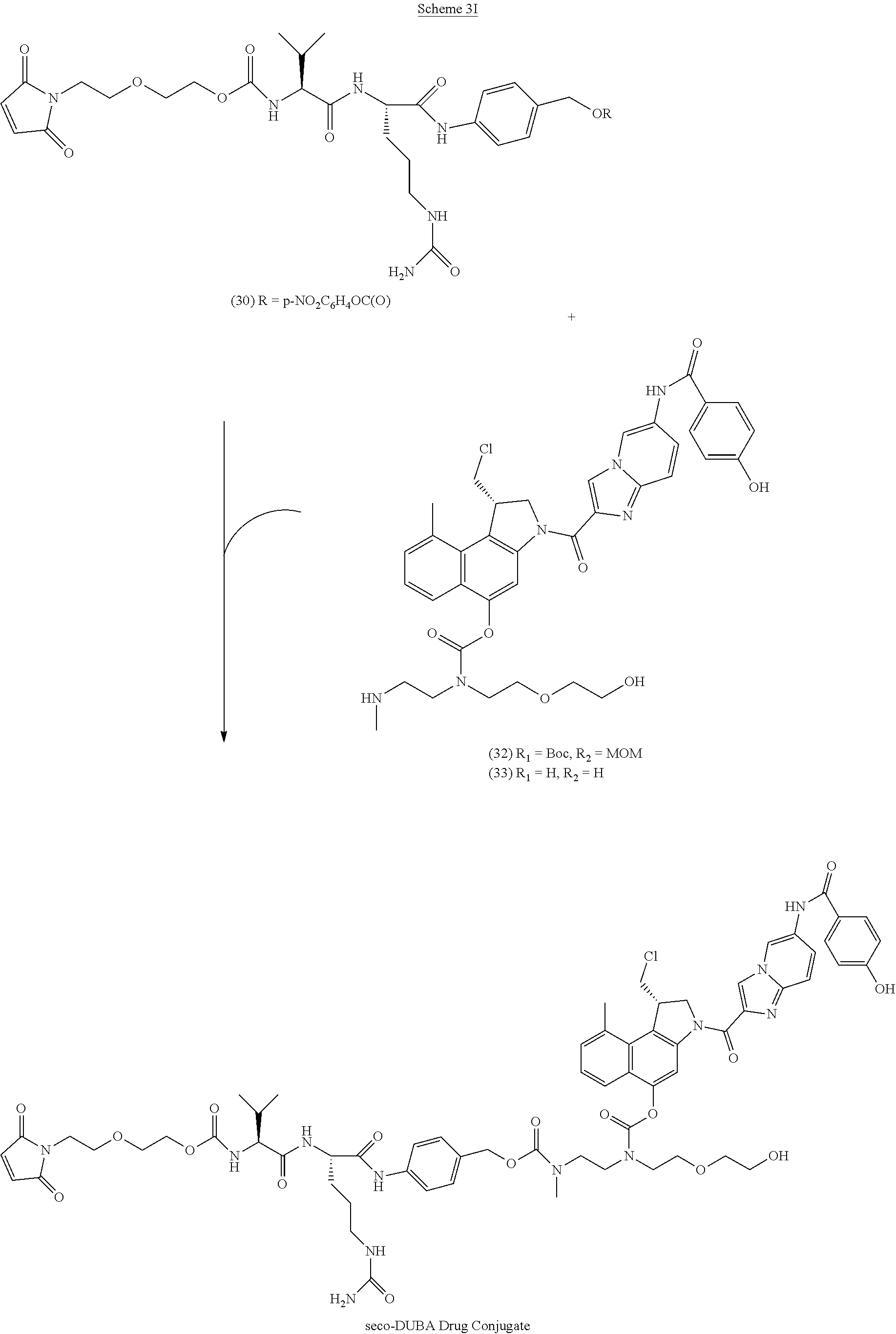

The process generates on average two free thiol groups per mAb leading to a statistical distribution of B7-H3-ADC with an average drug-to-antibody-ratio (DAR) of about two, and low amounts of high-molecular weight species and residual unconjugated duocarmycin moiety.

The order of the steps of the synthesis may be varied as desired. It is specifically contemplated that the method used will be that of Schemes 3A-3I, as described above.

### C. MGC018

In certain aspects, the B7-H3-ADC is MGC018. MGC018 comprises the Light Chain and Heavy Chain of anti-B7-H3 hmAb-A conjugated to a seco-DUBA payload. The amino acid sequences of the Ab, the cytotoxic duocarmycin moiety D, and the Linker molecule, LM, in MGC018 are shown below:
the Ab comprises:
(i) a light chain comprising the amino acid sequence of SEQ ID NO:19; and
(ii) a heavy chain comprising the amino acid sequence of SEQ ID NO:20; the D comprises seco-DUBA; and
   the LM comprises a Linker Molecule comprising a maleimide linker moiety, a valine-citrulline dipeptide linker, and a para-aminobenzyloxycarbonyl moiety.

### V. PD-1×CTLA-4 Bispecific Molecules

The PD-1×CTLA-4 bispecific molecules include bispecific molecules (e.g., bispecific antibodies, bispecific diabodies, etc.), chimeric or humanized antibodies, and such bispecific binding molecules having variant Fc Regions. PD-1×CTLA-4 bispecific molecules which may be used in the method of the present disclosure are disclosed, for example, in: WO 2014/209804; WO 2017/218707; WO 2017/193032; WO 2019/094637; and US 2019/0185569. Variants of such PD-1×CTLA-4 bispecific molecules may readily be generated, for example by incorporating alternative VH/VL Domains such as those provided herein. In certain embodiments, the PD-1×CTLA-4 bispecific molecule is lorgerlimab (also known as MGD019; the amino acid sequence of lorigerlimab is provided herein and also in WHO Drug Information, 2021, Recommended INN: List 125, 35(2):466-468). Additional PD-1×CTLA-4 bispecific molecules include vudalimab (CAS Reg. No.: 2329669-72-7, also known as XmAb20717, and XmAb® 717; Xencor, Inc.; the amino acid sequence of vudalimab is provided herein and also in WHO Drug Information, 2020, Recommended INN: List 123, 34(2):413-415); cadonilimab (CAS Reg. No.: 2394841-59-7, also known as AK104; Akeso Biopharma, Inc.; the amino acid sequence of cadonilimab is provided herein and also in WHO Drug Information, 2020, Recommended INN: List 124, 34(4):947-949) and MEDI5752 (Medimmune, Inc.) the amino acid sequence of MEDI5752 is provided in WO 2017/193032.

### A. Lorigerlimab

In certain embodiments, the PD-1×CTLA-4 bispecific molecule is lorigerlimab. Lorigerlimab is a bispecific, four chain, Fc Region-containing diabody having two binding sites specific for PD-1, two binding sites specific for CTLA-4, a variant IgG4 Fc Region engineered for extended half-life, and cysteine-containing E/K-coil Heterodimer-Promoting Domains. The amino acid sequence of lorigerlimab is shown below.

The first and third polypeptide chains of lorigerlimab comprise, in the N-terminal to C-terminal direction: an N-terminus, a VL Domain of a monoclonal antibody capable of binding to PD-1 (VL_{PD-1}) (SEQ ID NO:30; bolded and underlined in SEQ ID NO:28, below); an intervening linker peptide (Linker 1: GGGSGGGG (SEQ ID NO:10)); a VH Domain of a monoclonal antibody capable of binding to CTLA-4 (VH_{CTLA-4}) (SEQ ID NO:33; bolded and double underlined in SEQ ID NO:28, below); a cysteine-containing intervening linker peptide (Linker 2: GGCGGG (SEQ ID NO:11)); a cysteine-containing Heterodimer-Promoting (E-coil) Domain (EVAACEK-EVAALEK-EVAALEK-EVAALEK (SEQ ID NO:12)); an intervening linker peptide (Linker 3) comprising a stabilized IgG4 hinge region (SEQ ID NO:7); a variant IgG4 CH2-CH3 Domain comprising substitutions M252Y/S254T/T256E and lacking the C-terminal residue (SEQ ID NO:14); and a C-terminus.

The amino acid sequence of the first and third polypeptide chains of lorgerlimab is (SEQ ID NO:28):

EIVLTQSPAT LSLSPGERAT LSCRASESVD NYGMSFMNWF
QQKPGQPPKL LIHAASNQGS GVPSRFSGSG SGTDFTLTIS
SLEPEDFAVY FCQQSKEVPY TFGGGTKVEI KGGGSGGGGQ
VQLVESGGGV VQPGRSLRLS CAASGFTFSS YTMHWVRQAP
GKGLEWVTFI SYDGSNKHYA DSVKGRFTVS RDNSKNTLYL
QMNSLRAEDT AIYYCARTGW LGPFDYWGQG TLVTVSSGGC
GGGEVAACEK EVAALEKEVA ALEKEVAALE KESKYGPPCP
PCPAPEFLGG PSVFLFPPKP KDTLYITREP EVTCVVVDVS
QEDPEVQFNW YVDGVEVHNA KTKPREEQEN STYRVVSVLT
VLHQDWLNGK EYKCKVSNKG LPSSIEKTIS KAKGQPREPQ
VYTLPPSQEE MTKNQVSLTC LVKGFYPSDI AVEWESNGQP
ENNYKTTPPV LDSDGSFFLY SRLTVDKSRW QEGNVESCSV
MHEALHNHYT QKSLSLSLG

The second and fourth polypeptide chains of lorgerlimab comprise, in the N-terminal to C-terminal direction: an N-terminus, a VL Domain of a monoclonal antibody capable of binding to CTLA-4 (VL_{CTLA-4}) (SEQ ID NO:32; bolded and underlined in SEQ ID NO:29, below); an intervening linker peptide (Linker 1: GGGS GGGG (SEQ ID NO:10)); a VH Domain of a monoclonal antibody capable of binding PD-1 (VHPD-1) (SEQ ID NO:31; bolded and double underlined in SEQ ID NO:29, below); a cysteine-containing intervening linker peptide (Linker 2: GGCGGG (SEQ ID NO:10)); a cysteine-containing Heterodimer-Promoting (K-coil) Domain (KVAACKE-KVAALKE-KVAALKE-KVAALKE (SEQ ID NO:13)); and a C-terminus.

The amino acid sequence of the second and fourth polypeptide chains of lorigerlimab is (SEQ ID NO:29):

EIVLTQSPGT LSLSPGERAT LSCRASQSVS SSFLAWYQQK
PGQAPRLLIY GASSRATGIP DRESGSGSGT DFTLTISRLE
PEDFAVYYCQ QYGSSPWTFG QGTKVEIKGG GSGGGGQVQL
VQSGAEVKKP GASVKVSCKA SGYSFTSYWM NWVRQAPGQG
LEWIGVIHPS DSETWLDQKF KDRVTITVDK STSTAYMELS
SLRSEDTAVY YCAREHYGTS PFAYWGOGTL VTVSSGGCGG
GKVAACKEKV AALKEKVAAL KEKVAALKE

### B. Vudalimab

In certain embodiments, the PD-1×CTLA-4 bispecific molecule is vudalimab. The amino acid sequence of vudalimab is shown below.

The amino acid sequence of the first chain (heavy chain; anti-CTLA-4) of vudalimab is SE ID NO:34

EVQLVESGGG LVKPGGSLRL SCAASGFTES SYTMHWVRQA
PGKGLEWVSF ISYDGNNKYY ADSVKGRFTI SRDNAKNSLY
LQMNSLRAED TAVYYCARTG WLGPFDYWGQ GTLVTVSSAS
TKGPSVFPLA PSSKSTSGGT AALGCLVKDY FPEPVTVSWN
SGALTSGVHT FPAVLQSSGL YSLSSVVTVP SSSLGTQTYI
CNVNHKPSDT KVDKKVEPKS CDKTHTCPPC PAPPVAGPSV
FLFPPKPKDT LMISRTPEVT CVVVDVKHED PEVKENWYVD
GVEVHNAKTK PREEEYNSTY RVVSVLTVLH QDWLNGKEYK
CKVSNKALPA PIEKTISKAK GQPREPQVYT LPPSREEMTK
NQVSLTCDVS GFYPSDIAVE WESDGQPENN YKTTPPVLDS
DGSFFLYSKL TVDKSRWEQG DVESCSVLHE ALHSHYTQKS
LSLSPGK

The amino acid sequence of the second chain (light chain; anti-CTLA-4) of vudalimab is (SEQ ID NO:35):

EIVLTQSPGT LSLSPGERAT LSCRASQSVS SSYLAWYQQK
PGQAPRLLIY GAFSRATGIP DRESGSGSGT DETLTISRLE
PEDFAVYYCQ QYGSSPWTFG QGTKVEIKRT VAAPSVFIFP
PSDEQLKSGT ASVVCLLNNE YPREAKVQWK VDNALQSGNS
QESVTEQDSK DSTYSLSSTL TLSKADYEKH KVYACEVTHQ
GLSSPVTKSF NRGEC

The amino acid sequence of the third chain (scFv-h-CH2-CH3; anti-PD-1) of vudalimab is (SEQ ID NO:36):

EIVLTQSPAT LSASPGERVT LTCRASQSVG NDVAWYQQKP
GQAPRLLINY ASHRYTGVPD RFTGSGYGTE FTLTISSVQS
EDFGVYYCQQ DESSPRTFGG GTKVEIKGKP GSGKPGSGKP
GSGKPGSEVQ LVESGGGLVK PGGSLRLSCV ASGFTFSNYW
MNWVRQAPGK GLEWVAEIRL YSNNYATHYA ESVKGRETIS
RDDSKSTLYL QMNNLKTEDT GVYYCTRYYG NYGGYFDVWG
RGTLVTVSSE PKSSDKTHTC PPCPAPPVAG PSVELFPPKP
KDTLMISRTP EVTCVVVDVK HEDPEVKENW YVDGVEVHNA
KTKPREEQYN STYRVVSVLT VLHQDWLNGK EYKCKVSNKA
LPAPIEKTIS KAKGQPREPQ VYTLPPSREQ MTKNQVKLTC
LVKGFYPSDI AVEWESNGQP ENNYKTTPPV LDSDGSFFLY
SKLTVDKSRW QQGNVFSCSV LHEALHSHYT QKSLSLSPGK

### C. Cadonilimab

In certain embodiments, the PD-1×CTLA-4 bispecific molecule is cadonilimab. The amino acid sequence of cadonilimab is shown below.

The amino acid sequence of the first chain (heavy chain; anti-PD-1 and anti-CTLA-4 (scFv)) of cadonilimab is (SEQ ID NO:37):

EVQLVESGGG LVQPGGSLRL SCAASGFAFS SYDMSWVRQA
PGKGLDWVAT ISGGGRYTYY PDSVKGRFTI SRDNSKNNLY
LQMNSLRAED TALYYCANRY GEAWFAYWGQ GTLVTVSSAS
FPEPVTVSWN SGALTSGVHT FPAVLQSSGL YSLSSVVTVP
TKGPSVFPLA PSSKSTSGGT AALGCLVKDY SSSLGTQTYI
CNVNHKPSNT KVDKKVEPKS CDKTHTCPPC PAPEAAGAPS
VELFPPKPKD TLMISRTPEV TCVVVDVSHE DPEVKENWYV
DGVEVHNAKT KPREEQYNST YRVVSVLTVL HQDWLNGKEY
KCKVSNKALP APIEKTISKA KGQPREPQVY TLPPSRDELT
KNQVSLTCLV KGFYPSDIAV EWESNGQPEN NYKTTPPVLD
SDGSFFLYSK LTVDKSRWQQ GNVFSCSVMH EALHNHYTQK
SLSLSPGKGG GGSGGGGSGG GGSGGGGSQV QLVESGAEVK
KPGASVKVSC KASGYSFTGY TMNWVRQAPG QCLEWIGLIN
PYNNITNYAQ KFQGRVTFTV DTSISTAYME LSRLRSDDTG
VYFCARLDYR SYWGQGTLVT VSAGGGGSGG GGSGGGGSGG
GGSQAVVTQE PSLTVSPGGT VTLTCGSSTG AVTTSNFPNW
VQQKPGQAPR SLIGGTNNKA SWTPARFSGS LLGGKAALTI
SGAQPEDEAE YYCALWYSNH WVFGCGTKLT VLR

The amino acid sequence of the second chain (light chain; anti-PD-1) of cadonilimab is (SEQ ID NO:38):

DIQMTQSPSS MSASVGDRVT FTCRASQDIN TYLSWFQQKP
GKSPKTLIYR ANRLVSGVPS RESGSGSGQD YTLTISSLQP
EDMATYYCLQ YDEFPLTFGA GTKLELKRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSEN RGEC

### VI. Methods of Production

The binding molecules of the disclosure (e.g., anti-B7-H3 antibody hmAb-A and PD-1×CTLA-4 bispecific molecules) can be may be made recombinantly and expressed using any method known in the art for the production of recombinant proteins. For example, nucleic acids encoding the polypeptide chains of such binding molecules can be constructed, introduced into an expression vector, and expressed in suitable host cells. The binding molecules may be recombinantly produced in bacterial cells (e.g., E. coli cells), or eukaryotic cells (e.g., CHO, 293E, COS, NSO cells). In addition, the binding molecules can be expressed in a yeast cell such as Pichia, or Saccharomyces.

To produce the binding molecules (e.g., anti-B7-H3 antibody hmAb-A and PD-1×CTLA-4 bispecific molecules), one or more polynucleotides encoding the molecule may be constructed, introduced into an expression vector, and then expressed in suitable host cells. Standard molecular biology techniques are used to prepare the recombinant expression vector, transfect the host cells, select for transformants, culture the host cells and recover the molecules (See, for example, the techniques described in Green, M. R. et al., (2012), MOLECULAR CLONING, A LABORATORY MANUAL, 4th Ed., Cold Spring Harbor Laboratory, Cold Spring Harbor, NY and Ausubel et al. eds., 1998, CURRENT PROTOCOLS IN MOLECULAR BIOLOGY, John Wiley & Sons, NY). The expression vector(s) should have characteristics that permit replication of the vector in the host cell. The vector should also have promoter and signal sequences necessary for expression in the host cells. Such sequences are well known in the art. In addition to the nucleic acid sequence(s) encoding such binding molecules, the recombinant expression vectors may carry additional sequences, such as sequences that regulate replication of the vector in host cells (e.g., origins of replication) and selectable marker genes. Another method that may be employed is to express the gene sequence in plants (e.g., tobacco) or a transgenic animal. Suitable methods useful for expressing such binding molecules recombinantly in plants or milk have been disclosed (see, for example, Peeters et al. (2001) “Production Of Antibodies And Antibody Fragments In Plants,” Vaccine 19:2756; U.S. Pat. No. 5,849,992; and Pollock et al. (1999) “Transgenic Milk As A Method For The Production Of Recombinant Antibodies,” J. Immunol Methods 231:147-157).

Once a binding molecule has been recombinantly expressed, it may be purified from inside or outside (such as from culture media) of the host cell by any method known in the art for purification of polypeptides or polyproteins. Methods for isolation and purification commonly used for antibody purification (e.g., antibody purification schemes based on antigen selectivity) may be used for the isolation and purification of such molecules and are not limited to any particular method. For example, by for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, e.g., ion exchange, affinity, particularly by affinity for the specific antigen (optionally after Protein A selection where the PD-1×CTLA-4 bispecific molecule comprises an Fc Region), sizing column chromatography, hydrophobic, gel filtration, reverse-phase, and adsorption (Marshak et al. (1996) STRATEGIES FOR PROTEIN PURIFICATION AND CHARACTERIZATION: A Laboratory Course Manual. (Eds.), Cold Spring Harbor Laboratory Press, Cold Spring Harbor, NY).

### VII. Pharmaceutical Compositions

The B7-H3-ADC and PD-1×CTLA-4 binding molecules of the disclosure can be formulated as compositions. The compositions include bulk drug compositions useful in the manufacture of pharmaceutical compositions (e.g., impure or non-sterile compositions) and pharmaceutical compositions (i.e., compositions that are suitable for administration to a subject or patient) that can be used in the preparation of unit dosage forms. Such compositions comprise a prophylactically or therapeutically effective amount of a B7-H3-ADC, a PD-1×CTLA-4 binding molecule, or a combination thereof, and one or more pharmaceutically acceptable carrier(s) and may optionally additionally include one or more additional therapeutic agents. The pharmaceutical compositions may be supplied, for example, as an aqueous solution, or a dry lyophilized powder or water-free concentrate specifically adapted for reconstitution with such a pharmaceutically acceptable carrier or reconstituted with such a carrier.

As used herein, the term “pharmaceutically acceptable carrier” means a diluent, solvent, dispersion media, antibacterial and antifungal agents, excipient, or vehicle approved by a regulatory agency of the Federal or a state government or listed in the U.S. Pharmacopeia or other generally recognized pharmacopeia as being suitable for administration to animals, and more particularly to humans. Such pharmaceutical carriers can be sterile liquids, such as water and oils, including those of petroleum, animal, vegetable or synthetic origin. Saline solutions and aqueous dextrose and glycerol solutions can also be employed as liquid carriers, particularly for injectable solutions. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions are supplied either separately or mixed together in a dose form, for example, as a dry lyophilized powder or water-free concentrate, or as an aqueous solution in a hermetically sealed container such as a vial, ampoule or sachet indicating the quantity of active agent. Where the composition is to be administered by infusion, it can be dispensed with an infusion bottle containing sterile pharmaceutical grade water or saline. Where the composition is administered by injection, an ampoule of sterile water for injection, saline or other diluent can be provided so that the ingredients may be mixed prior to administration.

### VIII. Pharmaceutical Kits

The disclosure also provides a pharmaceutical pack or kit comprising one or more containers containing a pharmaceutical composition or pharmaceutical compositions and instructional material (e.g., a notice, package insert, instruction, etc.). Additionally, one or more other prophylactic or therapeutic agents useful for the treatment of a disease can also be included in the pharmaceutical kit. The containers of such pharmaceutical kits may, for example, comprise one or more hermetically sealed vials, ampoules, sachets, etc., indicating the quantity of active agent contained therein. Where the composition is to be administered by infusion, the container may be an infusion bottle, bag, etc. containing a sterile pharmaceutical-grade solution (e.g., water, saline, a buffer, etc.). Where the compositions are to be administered by injection, the pharmaceutical kit may contain an ampoule of sterile water, saline or other diluent for injection, so as to facilitate the mixing of the components of the pharmaceutical kit for administration to a subject (e.g., a human patient or other mammal). In certain embodiments, a pharmaceutical pack or kit comprises a B7-H3-ADC pharmaceutical composition and instructional material. In other embodiments, a pharmaceutical pack or kit comprises a B7-H3-ADC pharmaceutical composition, and a PD-1×CTLA-4 bispecific molecule composition, and instructional material.

In one embodiment, a B7-H3-ADC and/or PD-1×CTLA-4 bispecific molecule of such kit is/are supplied as a dry sterilized lyophilized powder or water-free concentrate in a hermetically sealed container and can be reconstituted, e.g., with water, saline, or other diluent to the appropriate concentration for administration to a subject. In another embodiment, a B7-H3-ADC and/or the PD-1×CTLA-4 bispecific molecule of such kit is supplied as an aqueous solution in a hermetically sealed container and can be diluted, e.g., with water, saline, or other diluent, to the appropriate concentration for administration to a subject. The kit can further comprise one or more other prophylactic and/or therapeutic agents useful for the treatment of cancer, in one or more containers; and/or the kit can further comprise one or more cytotoxic antibodies that bind one or more cancer antigens associated with cancer. In certain embodiments, the other prophylactic or therapeutic agent is a chemotherapeutic agent. In other embodiments, the prophylactic or therapeutic agent is a biological agent or hormonal therapeutic agent.

The included instructional material of the pharmaceutical kits may, for example, be of a content and format prescribed by a governmental agency regulating the manufacture, use or sale of pharmaceuticals or biological products, and may indicate approval by the agency of the manufacture, sale or use of the pharmaceutical composition for human administration and/or for human therapy. The instructional material may, for example provide information relating to the contained dose of the pharmaceutical composition, modes of how it may be prepared (e.g., reconstituted), and how it may be administered, etc. Such instructions may further provide information relating to the dose and administration of one or more pharmaceutical composition that are not provided in the kit.

Thus, for example, the included instructional material of the pharmaceutical kits may instruct that the provided pharmaceutical composition(s) is/are to be administered in combination with an additional agent which may be provided in the same pharmaceutical kit separately. Such instructional material may instruct that the B7-H3-ADC is to be administered at a dose of about 1 mg/kg to about 2 mg/kg, about 2 mg/kg to about 3 mg/kg, about 1 mg/kg to about 1.25 mg/kg, about 1.25 mg/kg to about 1.5 mg/kg, about 1.5 mg/kg to about 1.75 mg/kg, about 2 mg/kg to about 2.25 mg/kg, about 2.25 mg/kg to about 2.5 mg/kg, about 2.5 mg/kg to about 2.75 mg/kg, about 2.75 mg/kg to about 3 mg/kg, about 1 mg/kg, about 1.25 mg/kg, about 1.5 mg/kg, about 1.75 mg/kg, about 2 mg/kg, about 2.1 mg/kg, about 2.2 mg/kg, about 2.25 mg/kg, about 2.3 mg/kg, about 2.4 mg/kg, about 2.5 mg/kg, about 2.6 mg/kg, about 2.7 mg/kg, about 2.75 mg/kg, or about 3 mg/kg. Such instructional material may instruct that the B7-H3-ADC is to be administered about every 2 weeks, about every 3 weeks, about every 4 weeks, or more or less often. Such instructional material may instruct that the B7-H3-ADC pharmaceutical composition comprises a single dose, or more than one dose (e.g., 2 doses, 4 doses, 6 doses, 12 doses, 24 doses, etc.). Such instructional material may instruct that the provided B7-H3-ADC pharmaceutical composition comprises a fractionated dose, to be provided as two or more separate administrations within about 7+2 days of each other (e.g., 1 mg/kg given on day 1-3 and 1 mg/kg given on day 5-7 to provide a weekly dose of 2 mg/kg). Such instructional material may instruct that the provided B7-H3-ADC pharmaceutical composition comprises a fractionated dose, to be provided as two or more separate administrations within a 3-week or 4-week cycle.

Such instructional material may instruct that a PD-1×CTLA-4 bispecific molecule pharmaceutical composition is also administered. Such instructional material may instruct that the PD-1×CTLA-4 bispecific molecule pharmaceutical composition comprises lorigerlimab to be administered at a dose of about 1 mg/kg to about 6 mg/kg, about 1 mg/kg to about 3 mg/kg, or about 3 mg/kg to about 6 mg/kg, about 1 mg/kg, about 3 mg/kg, or about 6 mg/kg. Such instructional material may instruct that the PD-1×CTLA-4 bispecific molecule pharmaceutical composition comprises vudalimab to be administered at a dose of about 1 mg/kg to about 15 mg/kg, about 4 mg/kg to about 15 mg/kg, about 6 mg/kg to about 10 mg/kg, or about 10 mg/kg to about 15 mg/kg, about 4 mg/kg, about 6 mg/kg, about 10 mg/kg, or about 15 mg/kg. Such instructional material may instruct that the PD-1×CTLA-4 bispecific molecule pharmaceutical composition comprises cadonilimab to be administered at a dose of about 1 mg/kg to about 15 mg/kg, about 4 mg/kg to about 15 mg/kg, about 4 mg/kg to about 6 mg/kg, or about 6 mg/kg to about 15 mg/kg, about 4 mg/kg, about 6 mg/kg, about 10 mg/kg, or about 15 mg/kg. Such instructional material may instruct that the pharmaceutical composition comprises cadonilimab to be administered as a fixed dose of about 450 mg. Such instructional material may instruct that the PD-1×CTLA-4 bispecific molecule pharmaceutical composition comprises MEDI5752 to be administered at a dose of about 1 mg/kg to about 15 mg/kg, about 4 mg/kg to about 15 mg/kg, about 4 mg/kg to about 10 mg/kg, or about 6 mg/kg to about 15 mg/kg. Such instructional material may instruct that the provided PD-1×CTLA-4 bispecific molecule pharmaceutical composition comprises, or is to be reconstituted to comprise, a single dose, or more than one dose (e.g., 2 doses, 4 doses, 6 doses, 12 doses, 24 doses, etc.). Such instructional material may instruct that the PD-1×CTLA-4 bispecific molecule pharmaceutical composition is to be administered about every 2 weeks, about every 3 weeks, about every 4 weeks, or more or less often.

The included instructional material of the pharmaceutical kits may combine any set of such information (e.g., it may instruct that the B7-H3-ADC- is to be administered at a dose of about 1 mg/kg, and that such dose is to be administered about once a week for about 1 week, about 2 weeks, or about 3 weeks; it may instruct that the B7-H3-ADC is to be administered a dose of about 2 mg/kg, and that such dose is to be administered about every 3 weeks; it may instruct that the B7-H3-ADC is to be administered a dose of about 2.5 mg/kg, and that such dose is to be administered about every 3 weeks; it may instruct that the B7-H3-ADC is to be administered at a dose of about 3 mg/kg, and that such dose is to be administered about every 3 weeks; or is to be administered a dose of about 2 mg/kg, and that such dose is to be administered about every 4 weeks; or is to be administered a dose of about 2.5 mg/kg, and that such dose is to be administered about every 4 weeks; or is to be administered at a dose of about 3 mg/kg, and that such dose is to be administered about every 4 weeks; etc., and/or it may instruct that the PD-1×CTLA-4 bispecific molecule (e.g., lorigerlimab)—is to be administered at a dose of about 1 mg/kg, about 3 mg/kg, about 4 mg/kg, about 6 mg/kg, about 10 mg/kg, or about 15 mg/kg and that such dose is to be administered about every 2 weeks, about every 3 weeks, or every 4 weeks; etc.). Such instructional material may instruct regarding the mode of administration of the included pharmaceutical composition, for example that it is to be administered by intravenous (IV) infusion. The included instructional material of the pharmaceutical kits may instruct regarding the duration or timing of such administration, for example that the included pharmaceutical composition is to be administered by intravenous (IV) infusion over a period of about 30 minutes, about 45 minutes, about 60 minutes, about 75 minutes, a period of about 30-60 minutes, a period of 60-120 minutes, etc.

The included instructional material of the pharmaceutical kits may instruct regarding the appropriate or desired use of the included pharmaceutical composition(s), for example instructing that such pharmaceutical composition is to be administered for the treatment of cancer. Such cancer may be an adrenal gland cancer, an AIDS-associated cancer, an alveolar soft part sarcoma, an astrocytic tumor, an anal cancer, squamous cell carcinoma of the anal canal (SCAC), a bladder cancer, a bone cancer, a brain and spinal cord cancer, a metastatic brain tumor, a B-cell cancer, a breast cancer, a HER2⁺ breast cancer, triple negative breast cancer (TNBC), a carotid body tumors, a cervical cancer, a chondrosarcoma, a chordoma, a chromophobe renal cell carcinoma, a clear cell carcinoma, a colon cancer, a colorectal cancer (CRC), a non-microsatellite instability high colorectal cancer (non-MSI-H CRC), a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, a gastric cancer, a gestational trophoblastic disease, a germ cell tumor, a head and neck cancer, a glioblastoma, a hematological malignancy, a hepatocellular carcinoma, an islet cell tumor, a Kaposi's Sarcoma, a kidney cancer, a leukemia, an acute myeloid leukemia, a liposarcoma/malignant lipomatous tumor, a dedifferentiated liposarcoma, a liver cancer, a lymphoma, a lung cancer, a non-small-cell lung cancer (NSCLC), a medulloblastoma, a melanoma, a cutaneous melanoma, a meningioma, a mesothelioma pharyngeal cancer, a multiple endocrine neoplasia, a multiple myeloma, a myelodysplastic syndrome, a myxofibrosarcoma, a neuroblastoma, a neuroendocrine tumors, an ovarian cancer, a pancreatic cancer, a papillary thyroid carcinoma, a parathyroid tumor, a pediatric cancer, a peripheral nerve sheath tumor, a phaeochromocytoma, a pituitary tumor, a prostate cancer, a metastatic castration resistant prostate cancer (mCRPC), a posterior uveal melanoma, a renal cell cancer, a renal cell carcinoma (RCC), a renal metastatic cancer, a rhabdoid tumor, a rhabdomyosarcoma, a sarcoma, a skin cancer, a small round blue cell tumor of childhood, a neuroblastoma, a soft tissue sarcoma, a pleomorphic undifferentiated sarcoma, a squamous cell cancer, a squamous cell cancer of the head and neck (SCCHN), a stomach cancer, a synovial sarcoma, a testicular cancer, a thymic carcinoma, a thymoma, a thyroid cancer, a thyroid metastatic cancer, and a uterine cancer. In certain embodiments, the cancer is one in which B7-H3 is expressed.

### IX. Uses of a B7-H3-ADC of the Invention

A B7-H3-ADC and a PD-1×CTLA-4 bispecific molecule may be used to treat or prevent a variety of disorders, including cancer, including for example a cancer in which B7-H3 is expressed. Accordingly, the disclosure provides methods of treating cancer, such methods comprising administering a B7-H3-ADC in combination with a PD-1×CTLA-4 bispecific molecule to a subject in need thereof. In certain aspects, the disclosure provides methods of treating cancer, such methods comprising administering MGC018 in combination with lorigerlimab to a subject in need thereof. In certain aspects, the disclosure provides methods of treating cancer, such methods comprising administering MGC018 in combination with vudalimab to a subject in need thereof. In certain aspects, the disclosure provides methods of treating cancer, such methods comprising administering MGC018 in combination with cadonilimab to a subject in need thereof. In certain aspects, the disclosure provides methods of treating cancer, such methods comprising administering MGC018 in combination with MEDI5752 to a subject in need thereof. As used herein, the term “subject” refers to a human (i.e., a human patient) or other mammal. Non-limiting dosing regimens for administering such therapy to a subject in need thereof are provided herein.

The cancers that may be treated with a B7-H3-ADC in combination with a PD-1×CTLA-4 bispecific molecule include: an adrenal gland cancer, an AIDS-associated cancer, an alveolar soft part sarcoma, an astrocytic tumor, an anal cancer, squamous cell carcinoma of the anal canal (SCAC), a bladder cancer, a bone cancer, a brain and spinal cord cancer, a metastatic brain tumor, a B-cell cancer, a breast cancer, a HER2+ breast cancer, triple negative breast cancer (TNBC), a carotid body tumors, a cervical cancer, a chondrosarcoma, a chordoma, a chromophobe renal cell carcinoma, a clear cell carcinoma, a colon cancer, a colorectal cancer (CRC), a non-microsatellite instability high colorectal cancer (non-MSI-H CRC), a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, a gastric cancer, a gestational trophoblastic disease, a germ cell tumor, a head and neck cancer, a glioblastoma, a hematological malignancy, a hepatocellular carcinoma, an islet cell tumor, a Kaposi's Sarcoma, a kidney cancer, a leukemia, an acute myeloid leukemia, a liposarcoma/malignant lipomatous tumor, a dedifferentiated liposarcoma, a liver cancer, a lymphoma, a lung cancer, a non-small-cell lung cancer (NSCLC), a medulloblastoma, a melanoma, a cutaneous melanoma, a meningioma, a mesothelioma pharyngeal cancer, a multiple endocrine neoplasia, a multiple myeloma, a myelodysplastic syndrome, a myxofibrosarcoma, a neuroblastoma, a neuroendocrine tumors, an ovarian cancer, a pancreatic cancer, a papillary thyroid carcinoma, a parathyroid tumor, a pediatric cancer, a peripheral nerve sheath tumor, a phaeochromocytoma, a pituitary tumor, a prostate cancer, a metastatic castration resistant prostate cancer (mCRPC), a posterior uveal melanoma, a renal cell cancer, a renal cell carcinoma (RCC), a renal metastatic cancer, a rhabdoid tumor, a rhabdomyosarcoma, a sarcoma, a skin cancer, a small round blue cell tumor of childhood, a neuroblastoma, a soft tissue sarcoma, a pleomorphic undifferentiated sarcoma, a squamous cell cancer, a squamous cell cancer of the head and neck (SCCHN), a stomach cancer, a synovial sarcoma, a testicular cancer, a thymic carcinoma, a thymoma, a thyroid cancer, a thyroid metastatic cancer, and a uterine cancer. In certain embodiments the cancer expresses B7-H3.

In certain embodiments, a B7-H3-ADC may be used in combination with a PD-1×CTLA-4 bispecific molecule in the treatment of: anal cancer (including SCAC), breast cancer (including HER2+ breast cancer and/or TNBC), cervical cancer (including HPV-related cervical cancer and cervical squamous cell carcinoma) colorectal cancer (including, non-microsatellite instability high colorectal cancer (non-MSI-H CRC), head and neck cancer (including HPV-related head and neck cancer and SCCHN), kidney cancer (including renal cancer and renal cell carcinoma), liver cancer (including hepatocellular carcinoma), lung cancer (including NSCLC), melanoma (including cutaneous melanoma and posterior uveal melanoma), ovarian cancer, pancreatic cancer, prostate cancer (including mCRPC), soft tissue sarcoma (including dedifferentiated liposarcoma, myxofibrosarcoma, pleomorphic undifferentiated sarcoma, and synovial sarcoma), and squamous cell cancer. In certain embodiments the cancer expresses B7-H3.

In certain embodiments, a B7-H3-ADC in combination with a PD-1×CTLA-4 bispecific molecule of the present disclosure is administered as a first-line therapy for treatment of cancer. In certain embodiments, a B7-H3-ADC in combination with a PD-1×CTLA-4 bispecific molecule of the present disclosure is administered after one or more prior lines of therapy. In certain embodiments, a B7-H3-ADC in combination with a PD-1×CTLA-4 bispecific molecule of the present disclosure may be employed as an adjuvant therapy at the time of, or after surgical removal of a tumor in order to delay, suppress or prevent the development of metastasis. A B7-H3-ADC in combination with a PD-1×CTLA-4 bispecific molecule of the present disclosure may also be administered before surgery (e.g., as a neoadjuvant therapy) in order to decrease the size of the tumor and thus enable or simplify such surgery, spare tissue during such surgery, and/or decrease any resulting disfigurement.

The disclosure specifically encompasses administering a B7-H3-ADC in combination with a PD-1×CTLA-4 bispecific molecule, and in further combination with one or more other therapies known to those skilled in the art for the treatment or prevention of cancer, including but not limited to, current standard and experimental chemotherapies, hormonal therapies, biological therapies, immunotherapies, radiation therapies, or surgery. In some embodiments, a B7-H3-ADC in combination with a PD-1×CTLA-4 bispecific molecule, may be administered in further combination with a therapeutically or prophylactically effective amount of one or more therapeutic agents or chemotherapeutic agents known to those skilled in the art for the treatment and/or prevention of cancer, in particular a B7-H3-expressing cancer. Therapeutic agents and chemotherapeutic agents commonly used in the treatment of B7-H3-expressing cancers include, but are not limited to platinum-based chemotherapeutics (particularly, carboplatin, oxaliplatin, and carboplatin), taxanes (particularly, docetaxel and paclitaxel), hormonal therapies (particularly, abiraterone and enzalutamide), anthracyclines (particularly, daunorubicin, doxorubicin, and epirubicin), capecitabine, cyclophosphamide, leucovorin, methotrexate, radium 223, sipuleucel-T, 5-fluorouracil (5-FU), and immune checkpoint inhibitors (such as, retifanlimab and tebotelimab).

As used herein, the term “combination” refers to the use of more than one therapeutic agent. The use of the term “combination” does not restrict the order in which therapeutic agents are administered to a subject (e.g., a human patient or other mammal) with a disorder, nor does it mean that the agents are administered at exactly the same time. The term combination means that a B7-H3-ADC, a PD-1×CTLA-4 bispecific molecule, and any other agent are administered to a human patient or other mammal in a sequence and within a time interval such that the combination of a B7-H3-ADC, the PD-1×CTLA-4 bispecific molecule and the other agent provide an increased benefit than if they were administered otherwise. For example, each therapeutic agent (e.g., chemotherapy, radiation therapy, hormonal therapy or biological therapy) may be administered at the same time or sequentially in any order at different points in time; however, if not administered at the same time, they should be administered sufficiently close in time so as to provide the desired therapeutic or prophylactic effect. Each therapeutic agent can be administered separately, in any appropriate form and by any suitable route, e.g., one by the oral route and one parenterally, etc. Non-limiting dosing regimens for administering a B7-H3-ADC in combination with a PD-1×CTLA-4 bispecific molecule to a subject in need thereof are provided herein.

### X. Methods of Administration and Dose

A binding molecule of the disclosure (e.g., a B7-H3-ADC and/or a PD-1×CTLA-4 bispecific molecule) can be administered by a variety of methods to a subject, e.g., a subject in need thereof, for example a human patient. For many applications, the route of administration is one of: intravenous injection or infusion (IV), subcutaneous injection (SC), intraperitoneally (IP), or intramuscular injection. It is also possible to use intra-articular delivery. Other modes of parenteral administration can also be used. Examples of such modes include: intraarterial, intrathecal, intracapsular, intraorbital, intracardiac, intradermal, transtracheal, subcuticular, intraarticular, subcapsular, subarachnoid, intraspinal, and epidural and intrasternal injection.

The molecules (e.g., a B7-H3-ADC, and/or PD-1×CTLA-4 bispecific molecule) can be administered as a weight-based dose (e.g., 3.5 mg/kg). The dose can also be selected to reduce or avoid production of antibodies against the administered molecules. Dosage regimens are adjusted to provide the desired response, e.g., a therapeutic response or a combinatorial therapeutic effect. Generally, doses of a B7-H3-ADC and a PD-1×CTLA-4 bispecific molecule (and optionally a further agent) can be used in order to provide a subject with the agent in bioavailable quantities. As used herein, the term “dose” refers to a specified amount of medication taken at one time. The term “dosage” refers to the administering of a specific amount, number, and frequency of doses over a specified period of time; the term dosage thus includes chronological features, such as duration and periodicity.

The term “weight-based dose” as used herein, refers to a discrete amount of a molecule to be administered per a unit of patient weight, for example milligrams of drug per kilograms of a subject's body weight (mg/kg body weight, abbreviated herein as “mg/kg”). The calculated dose will be administered based on the subject's body weight at baseline. The term “flat dose,” as used herein, refers to a dose that is independent of the weight of the patient, and includes physically discrete units of a molecule that are suited as a unitary dose for the subjects to be treated; wherein each unit contains a predetermined quantity of a drug. Typically, a significant (≥10%) change in body weight from baseline or established plateau weight will generally prompt recalculation of dose. Single or multiple doses may be given. Compositions comprising a B7-H3-ADC and/or a PD-1×CTLA-4 bispecific molecule may be administered to a subject in need thereof via infusion.

The term “fractionated dose” as used herein, refers to two or more separate administrations of a molecule to be administered to achieve a particular desired dose. A fractionated dose provides that the desired dose will be fractionated into two or more separate administrations. The dose may be fractionated equally and/or unequally between such two or more administrations. The fractionated dose can be two or more separate administrations within about 7+2 days. In certain embodiments, the fractionated dose can be two or more separate administrations within a cycle (e.g., a 3-week or 4-week cycle). By way of example, and not to be construed as limiting, a desired dose of 2 mg/kg, a subject can be provided a dose of 1 mg/kg as a first administration (on week 1) and a dose of 1 mg/kg as a second administration (on one of weeks 2-4), a 1 mg/kg dose may be fractionated into two separate administrations of 0.5 mg/kg within about 7±2 days, a 1 mg/kg dose may be fractionated into two separate administrations of 0.25 mg/kg and 0.75 mg/kg within about 7±2 days, a 1.5 mg/kg dose may be fractionated into three separate administrations of 0.5 mg/kg within about 7±2 days, or a 1.5 mg/kg dose may be fractionated into three separate administrations of 0.25 mg/kg, 0.25 mg/kg, and 1.0 mg/kg within about 7±2 days. In an embodiment, a B7-H3-ADC is administered at a fractionated dose of 1 mg/kg on day 1-3 and 1 mg/kg on day 5-7 to achieve a dose of 2 mg/kg. In another embodiment, a B7-H3-ADC is administered at a fractionated dose of 1.5 mg/kg on day 1-3 and 1.5 mg/kg on day 5-7 to achieve a dose of 3 mg/kg. In an embodiment, for a desired dose of 2 mg/kg in a 3-week cycle, 1 mg/kg can be given on week 1 and 1 mg/kg can be given on week 2 of a 3-week cycle, with no administration on week 3. In another embodiment, for a desired dose of 2 mg/kg in a 4-week cycle, 1 mg/kg can be given on week 1 and 1 mg/kg can be given on week 2 of a 4-week cycle, with no administration on weeks 3 and 4. In another embodiment, for a desired dose of 3 mg/kg in a 3-week cycle, 1 mg/kg can be given on each week of a 3-week cycle. In another embodiment, for a desired dose of 3 mg/kg in a 4-week cycle, 1 mg/kg can be given on weeks 1-3 of a 4-week cycle, with no administration on week 4.

In certain embodiments, a B7-H3-ADC is administered to a subject in need thereof at a weight-based dose of about 1 mg/kg to about 2 mg/kg, about 2 mg/kg to about 3 mg/kg, about 1 mg/kg to about 1.25 mg/kg, about 1.25 mg/kg to about 1.5 mg/kg, about 1.5 mg/kg to about 1.75 mg/kg, about 2 mg/kg to about 2.25 mg/kg, about 2.25 mg/kg to about 2.5 mg/kg, about 2.5 mg/kg to about 2.75 mg/kg, about 2.75 mg/kg to about 3 mg/kg, about 1 mg/kg, about 1.25 mg/kg, about 1.5 mg/kg, about 1.75 mg/kg, about 2 mg/kg, about 2.1 mg/kg, about 2.2 mg/kg, about 2.25 mg/kg, about 2.3 mg/kg, about 2.4 mg/kg, about 2.5 mg/kg, about 2.6 mg/kg, about 2.7 mg/kg, about 2.75 mg/kg, or about 3 mg/kg. In certain embodiments, a B7-H3-ADC is to be administered about every 1 week, about every 2 weeks, about every 3 weeks, about every 4 weeks, or more or less often. In certain embodiments, a dose of B7-H3-ADC is provided as a single dose. In certain embodiments, a dose of B7-H3-ADC is provided as a fractionated dose of two or more separate administrations. In certain embodiments, the B7-H3-ADC that is administered is MGC018.

In certain embodiments, the PD-1×CTLA-4 bispecific molecule is lorigerlimab and is administered to a subject in need thereof at a weight-based dose of about 1 mg/kg to about 15 mg/kg. In certain embodiments the PD-1×CTLA-4 bispecific molecule is lorgerlimab, vudalimab, cadonilimab, or MEDI5752. In certain embodiments, lorgerlimab is administered to a subject in need thereof at dose of about 1 mg/kg, about 3 mg/kg, or about 6 mg/kg. In certain embodiments, lorigerlimab is administered to a subject in need thereof at a dose of about 1 mg/kg. In certain embodiments, lorigerlimab is administered to a subject in need thereof at a dose of about 3 mg/kg. In certain embodiments, lorigerlimab is administered to a subject in need thereof at a dose of about 6 mg/kg. In certain embodiments, lorigerlimab is to be administered about every 2 weeks, about every 3 weeks, about every 4 weeks, or more or less often. In certain embodiments, lorigerlimab is administered to a subject in need thereof at a dose of about 6 mg/kg every 3 weeks. In certain embodiments, lorigerlimab is administered to a subject in need thereof at a dose of about 6 mg/kg every 4 weeks. In certain embodiments, vudalimab is administered to a subject in need thereof at dose of about 10 mg/kg or about 15 mg/kg. In certain embodiments, vudalimab is administered to a subject in need thereof at dose of about 10 mg/kg. In certain embodiments, vudalimab is administered to a subject in need thereof at dose of about 15 mg/kg. In certain embodiments, vudalimab is to be administered about every 2 weeks, about every 3 weeks, about every 4 weeks, or more or less often. In certain embodiments, vudalimab is administered to a subject in need thereof at a dose of about 10 mg/kg every 2 weeks. In certain embodiments, vudalimab is administered to a subject in need thereof at a dose of about 15 mg/kg every 2 weeks. In certain embodiments, cadonilimab is administered to a subject in need thereof at dose of about 6 mg/kg or about 15 mg/kg. In certain embodiments, cadonilimab is administered to a subject in need thereof at dose of about 6 mg/kg. In certain embodiments, cadonilimab is administered to a subject in need thereof at dose of about 15 mg/kg. In certain embodiments, cadonilimab is to be administered about every 2 weeks, about every 3 weeks, about every 4 weeks, or more or less often. In certain embodiments, cadonilimab is administered to a subject in need thereof at a dose of about 6 mg/kg every 2 weeks. In certain embodiments, cadonilimab is administered to a subject in need thereof at a dose of about 15 mg/kg every 3 weeks.

With respect to weight-based doses, the term “about” is intended to denote a range that is ±10% of a recited dose, such that for example, a dose of about 1 mg/kg will be between 0.09 mg/kg and 1.01 mg/kg.

The terms “dosing interval” and “dosing intervals” as used herein, refer to the time interval between doses, which can be regular or intermittent. A dosage of a molecule (e.g., a dose of a B7-H3-ADC and/or a dose of a PD-1×CTLA-4 bispecific molecule) can be administered at a periodic dosing intervals over a period of time sufficient to encompass at least 2 doses, at least 4 doses, at least 6 doses, at least 12 doses, or at least 24 doses (a course of treatment). For example, a dosage may be administered e.g., once or twice daily, or about one to four times per week, or particularly once every week (“Q1W”), once every two weeks (“Q2W”), once every three weeks (“Q3W”), once every four weeks (“Q4W”), etc. A desired dose may be provided in a single administration or as a fractionated dose of two or more separate administrations within a given cycle (e.g., 3-week or 4-week cycle). Such periodic administration may continue for a period of time e.g., for between about 1 to 52 weeks, or for more than 52 weeks. Such course of treatment may be divided into increments, each referred to herein as a “cycle,” of e.g., between 2 to 8 weeks, between about 3 to 7 weeks, about 3 weeks, about 4 weeks, about 6 weeks, or about 8 weeks, during which a set number of doses are administered. The dose and/or the frequency of administration may be the same or different during each cycle. Factors that may influence the dosage and timing required to effectively treat a subject, include, e.g., the severity of the disease or disorder, formulation, route of delivery, previous treatments, the general health and/or age of the subject, and the presence of other diseases in the subject. Moreover, treatment of a subject with a therapeutically effective amount of a compound can include a single treatment or can include a series of treatments.

A “dosing regimen” is a dosage administration in which a patient is administered a predetermined dose (or set of such predetermined doses) at a predetermined frequency (or set of such frequencies) for a predetermined periodicity (or periodicities). In an embodiment, a dosing regimen comprises administration of a B7-H3-ADC at a dose of about 1 mg/kg to about 3 mg/kg every 3 weeks. In an embodiment, a dosing regimen comprises administration of a B7-H3-ADC at a weight-based dose of about 1 mg/kg to about 2 mg/kg every 3 weeks. In an embodiment, a dosing regimen comprises administration of a B7-H3-ADC at a dose of about 2 mg/kg to about 3 mg/kg every 3 weeks. In an embodiment, a dosing regimen comprises administration of a B7-H3-ADC at a dose of about 1 mg/kg every 3 weeks. In an embodiment, a dosing regimen comprises administration of a B7-H3-ADC at a dose of about 1.25 mg/kg every 3 weeks. In an embodiment, a dosing regimen comprises administration of a B7-H3-ADC at a dose of about 1.5 mg/kg every 3 weeks. In an embodiment, a dosing regimen comprises administration of a B7-H3-ADC at a dose of about 1.75 mg/kg every 3 weeks. In an embodiment, a dosing regimen comprises administration of a B7-H3-ADC at a dose of about 2 mg/kg every 3 weeks. In an embodiment, a dosing regimen comprises administration of a B7-H3-ADC at a dose of about 2.1 mg/kg every 3 weeks. In an embodiment, a dosing regimen comprises administration of a B7-H3-ADC at a dose of about 2.2 mg/kg every 3 weeks. In an embodiment, a dosing regimen comprises administration of a B7-H3-ADC at a dose of about 2.25 mg/kg every 3 weeks. In an embodiment, a dosing regimen comprises administration of a B7-H3-ADC at a dose of about 2.3 mg/kg every 3 weeks. In an embodiment, a dosing regimen comprises administration of a B7-H3-ADC at a dose of about 2.4 mg/kg every 3 weeks. In an embodiment, a dosing regimen comprises administration of a B7-H3-ADC at a dose of about 2.5 mg/kg every 3 weeks. In an embodiment, a dosing regimen comprises administration of a B7-H3-ADC at a dose of about 2.6 mg/kg every 3 weeks. In an embodiment, a dosing regimen comprises administration of a B7-H3-ADC at a dose of about 2.7 mg/kg every 3 weeks. In an embodiment, a dosing regimen comprises administration of a B7-H3-ADC at a dose of about 2.75 mg/kg every 3 weeks. In an embodiment, a dosing regimen comprises administration of a B7-H3-ADC at a dose of about 3 mg/kg every 3 weeks.

In an embodiment, a dosing regimen comprises administration of a B7-H3-ADC at a dose of about 1 mg/kg to about 3 mg/kg every 4 weeks. In an embodiment, a dosing regimen comprises administration of a B7-H3-ADC at a dose of about 1 mg/kg to about 2 mg/kg every 4 weeks. In an embodiment, a dosing regimen comprises administration of a B7-H3-ADC at a dose of about 2 mg/kg to about 3 mg/kg every 4 weeks. In an embodiment, a dosing regimen comprises administration of a B7-H3-ADC at a dose of about 1 mg/kg every 4 weeks. In an embodiment, a dosing regimen comprises administration of a B7-H3-ADC at a dose of about 1.25 mg/kg every 4 weeks. In an embodiment, a dosing regimen comprises administration of a B7-H3-ADC at a dose of about 1.5 mg/kg every 4 weeks. In an embodiment, a dosing regimen comprises administration of a B7-H3-ADC at a dose of about 1.75 mg/kg every 4 weeks. In an embodiment, a dosing regimen comprises administration of a B7-H3-ADC at a dose of about 2 mg/kg every 4 weeks. In an embodiment, a dosing regimen comprises administration of a B7-H3-ADC at a dose of about 2.1 mg/kg every 4 weeks. In an embodiment, a dosing regimen comprises administration of a B7-H3-ADC at a dose of about 2.2 mg/kg every 4 weeks. In an embodiment, a dosing regimen comprises administration of a B7-H3-ADC at a dose of about 2.25 mg/kg every 4 weeks. In an embodiment, a dosing regimen comprises administration of a B7-H3-ADC at a dose of about 2.3 mg/kg every 4 weeks. In an embodiment, a dosing regimen comprises administration of a B7-H3-ADC at a dose of about 2.4 mg/kg every 4 weeks. In an embodiment, a dosing regimen comprises administration of a B7-H3-ADC at a dose of about 2.5 mg/kg every 4 weeks. In an embodiment, a dosing regimen comprises administration of a B7-H3-ADC at a dose of about 2.6 mg/kg every 4 weeks. In an embodiment, a dosing regimen comprises administration of a B7-H3-ADC at a dose of about 2.7 mg/kg every 4 weeks. In an embodiment, a dosing regimen comprises administration of a B7-H3-ADC at a dose of about 2.75 mg/kg every 4 weeks. In an embodiment, a dosing regimen comprises administration of a B7-H3-ADC at a dose of about 3 mg/kg every 4 weeks.

In an embodiment, a dosing regimen comprises administration of a B7-H3-ADC at a dose of about 1 mg/kg to about 3 mg/kg every 3 weeks, or every 4 weeks, and lorigerlimab at dose of about 1 mg/kg to about 6 mg/kg administered every 2 weeks, every 3 weeks, or every 4 weeks. In an embodiment, a dosing regimen comprises administration of a B7-H3-ADC at a dose of about 1 mg/kg to about 3 mg/kg every 3 weeks or 4 weeks and vudalimab at dose of about 6 mg/kg to about 15 mg/kg administered every 2 weeks, every 3 weeks, or every 4 weeks. In an embodiment, a dosing regimen comprises administration of a B7-H3-ADC at a dose of about 1 mg/kg to about 3 mg/kg every 3 weeks or 4 weeks and cadonilimab at dose of about 6 mg/kg to about 15 mg/kg administered every 2 weeks, every 3 weeks, or every 4 weeks. In an embodiment, a dosing regimen comprises administration of a B7-H3-ADC at a dose of about 1 mg/kg to about 3 mg/kg every 3 weeks or 4 weeks and MEDI5752 at dose of about 1 mg/kg to about 15 mg/kg administered every 2 weeks, every 3 weeks, or every 4 weeks.

In an embodiment, a dosing regimen comprises administration of a B7-H3-ADC at a dose of about 1 mg/kg to about 3 mg/kg every 3 weeks and lorigerlimab at a dose of about 1 mg/kg to about 6 mg/kg administered every 3 weeks. In an embodiment, a dosing regimen comprises administration of a B7-H3-ADC at a dose of about 1 mg/kg to about 2 mg/kg every 3 weeks and lorigerlimab at a dose of about 3 mg/kg to about 6 mg/kg administered every 3 weeks. In an embodiment, a dosing regimen comprises administration of a B7-H3-ADC at a dose of about 2 mg/kg to about 3 mg/kg every 3 weeks and lorigerlimab at a dose of about 3 mg/kg to about 6 mg/kg administered every 3 weeks. In an embodiment, a dosing regimen comprises administration of a B7-H3-ADC at a dose of about 1 mg/kg every 3 weeks and lorigerlimab at a dose of about 3 mg/kg to about 6 mg/kg every 3 weeks. In an embodiment, a dosing regimen comprises administration of a B7-H3-ADC at a dose of about 1.25 mg/kg every 3 weeks and lorigerlimab at a dose of about 3 mg/kg to 6 mg/kg every 3 weeks. In an embodiment, a dosing regimen comprises administration of a B7-H3-ADC at a dose of about 1.5 mg/kg every 3 weeks and lorigerlimab at a dose of about 3 mg/kg to 6 mg/kg every 3 weeks. In an embodiment, a dosing regimen comprises administration of a B7-H3-ADC at a dose of about 1.75 mg/kg every 3 weeks and lorigerlimab at a dose of about 3 mg/kg to 6 mg/kg every 3 weeks. In an embodiment, a dosing regimen comprises administration of a B7-H3-ADC at a dose of about 2 mg/kg every 3 weeks and lorigerlimab at a dose of about 3 mg/kg to 6 mg/kg every 3 weeks. In an embodiment, a dosing regimen comprises administration of a B7-H3-ADC at a dose of about 2.1 mg/kg every 3 weeks and lorigerlimab at a dose of about 3 mg/kg to 6 mg/kg every 3 weeks. In an embodiment, a dosing regimen comprises administration of a B7-H3-ADC at a dose of about 2.2 mg/kg every 3 weeks and lorigerlimab at a dose of about 3 mg/kg to 6 mg/kg every 3 weeks. In an embodiment, a dosing regimen comprises administration of a B7-H3-ADC at a dose of about 2.25 mg/kg every 3 weeks and lorigerlimab at a dose of about 3 mg/kg to 6 mg/kg every 3 weeks. In an embodiment, a dosing regimen comprises administration of a B7-H3-ADC at a dose of about 2.3 mg/kg every 3 weeks and lorigerlimab at a dose of about 3 mg/kg to 6 mg/kg every 3 weeks. In an embodiment, a dosing regimen comprises administration of a B7-H3-ADC at a dose of about 2.4 mg/kg every 3 weeks and lorigerlimab at a dose of about 3 mg/kg to 6 mg/kg every 3 weeks. In an embodiment, a dosing regimen comprises administration of a B7-H3-ADC at a dose of about 2.5 mg/kg every 3 weeks and lorigerlimab at a dose of about 3 mg/kg to 6 mg/kg every 3 weeks. In an embodiment, a dosing regimen comprises administration of a B7-H3-ADC at a dose of about 2.6 mg/kg every 3 weeks and lorigerlimab at a dose of about 3 mg/kg to 6 mg/kg every 3 weeks. In an embodiment, a dosing regimen comprises administration of a B7-H3-ADC at a dose of about 2.7 mg/kg every 3 weeks and lorigerlimab at a dose of about 3 mg/kg to 6 mg/kg every 3 weeks. In an embodiment, a dosing regimen comprises administration of a B7-H3-ADC at a dose of about 2.75 mg/kg every 3 weeks and lorigerlimab at a dose of about 3 mg/kg to 6 mg/kg every 3 weeks. In an embodiment, a dosing regimen comprises administration of a B7-H3-ADC at a dose of about 3 mg/kg every 3 weeks and lorigerlimab at a dose of about 3 mg/kg to 6 mg/kg every 3 weeks.

In an embodiment, a dosing regimen comprises administration of a B7-H3-ADC at a dose of about 1 mg/kg every 3 weeks and lorigerlimab at a dose of about 3 mg/kg every 3 weeks. In an embodiment, a dosing regimen comprises administration of a B7-H3-ADC at a dose of about 1 mg/kg every 3 weeks and lorigerlimab at a dose of about 6 mg/kg every 3 weeks.

In an embodiment, a dosing regimen comprises administration of a B7-H3-ADC at a dose of about 1.25 mg/kg every 3 weeks and lorigerlimab at a dose of about 3 mg/kg every 3 weeks. In an embodiment, a dosing regimen comprises administration of a B7-H3-ADC at a dose of about 1.25 mg/kg every 3 weeks and lorigerlimab at a dose of about 6 mg/kg every 3 weeks.

In an embodiment, a dosing regimen comprises administration of a B7-H3-ADC at a dose of about 1.5 mg/kg every 3 weeks and lorigerlimab at a dose of about 3 mg/kg every 3 weeks. In an embodiment, a dosing regimen comprises administration of a B7-H3-ADC at a dose of about 1.5 mg/kg every 3 weeks and lorigerlimab at a dose of about 6 mg/kg every 3 weeks.

In an embodiment, a dosing regimen comprises administration of a B7-H3-ADC at a dose of about 1.75 mg/kg every 3 weeks and lorigerlimab at a dose of about 3 mg/kg every 3 weeks. In an embodiment, a dosing regimen comprises administration of a B7-H3-ADC at a dose of about 1.75 mg/kg every 3 weeks and lorigerlimab at a dose of about 6 mg/kg every 3 weeks.

In an embodiment, a dosing regimen comprises administration of a B7-H3-ADC at a dose of about 2 mg/kg every 3 weeks and lorigerlimab at a dose of about 3 mg/kg every 3 weeks. In an embodiment, a dosing regimen comprises administration of a B7-H3-ADC at a dose of about 2 mg/kg every 3 weeks and lorigerlimab at a dose of about 6 mg/kg every 3 weeks.

In an embodiment, a dosing regimen comprises administration of a B7-H3-ADC at a dose of about 2.1 mg/kg every 3 weeks and lorigerlimab at a dose of about 3 mg/kg every 3 weeks. In an embodiment, a dosing regimen comprises administration of a B7-H3-ADC at a dose of about 2.1 mg/kg every 3 weeks and lorigerlimab at a dose of about 6 mg/kg every 3 weeks.

In an embodiment, a dosing regimen comprises administration of a B7-H3-ADC at a dose of about 2.2 mg/kg every 3 weeks and lorigerlimab at a dose of about 3 mg/kg every 3 weeks. In an embodiment, a dosing regimen comprises administration of a B7-H3-ADC at a dose of about 2.2 mg/kg every 3 weeks and lorigerlimab at a dose of about 6 mg/kg every 3 weeks.

In an embodiment, a dosing regimen comprises administration of a B7-H3-ADC at a dose of about 2.25 mg/kg every 3 weeks and lorigerlimab at a dose of about 3 mg/kg every 3 weeks. In an embodiment, a dosing regimen comprises administration of a B7-H3-ADC at a dose of about 2.25 mg/kg every 3 weeks and lorigerlimab molecule at a dose of about 6 mg/kg every 3 weeks.

In an embodiment, a dosing regimen comprises administration of a B7-H3-ADC at a dose of about 2.3 mg/kg every 3 weeks and lorigerlimab at a dose of about 3 mg/kg every 3 weeks. In an embodiment, a dosing regimen comprises administration of a B7-H3-ADC at a dose of about 2.3 mg/kg every 3 weeks and lorigerlimab at a dose of about 6 mg/kg every 3 weeks.

In an embodiment, a dosing regimen comprises administration of a B7-H3-ADC at a dose of about 2.4 mg/kg every 3 weeks and lorigerlimab at a dose of about 3 mg/kg every 3 weeks. In an embodiment, a dosing regimen comprises administration of a B7-H3-ADC at a dose of about 2.4 mg/kg every 3 weeks and lorigerlimab at a dose of about 6 mg/kg every 3 weeks.

In an embodiment, a dosing regimen comprises administration of a B7-H3-ADC at a dose of about 2.5 mg/kg every 3 weeks and lorigerlimab at a dose of about 3 mg/kg every 3 weeks. In an embodiment, a dosing regimen comprises administration of a B7-H3-ADC at a dose of about 2.5 mg/kg every 3 weeks and lorigerlimab at a dose of about 6 mg/kg every 3 weeks.

In an embodiment, a dosing regimen comprises administration of a B7-H3-ADC at a dose of about 2.6 mg/kg every 3 weeks and lorigerlimab at a dose of about 3 mg/kg every 3 weeks. In an embodiment, a dosing regimen comprises administration of a B7-H3-ADC at a dose of about 2.6 mg/kg every 3 weeks and lorigerlimab at a dose of about 6 mg/kg every 3 weeks.

In an embodiment, a dosing regimen comprises administration of a B7-H3-ADC at a dose of about 2.7 mg/kg every 3 weeks and lorigerlimab at a dose of about 3 mg/kg every 3 weeks. In an embodiment, a dosing regimen comprises administration of a B7-H3-ADC at a dose of about 2.7 mg/kg every 3 weeks and lorigerlimab at a dose of about 6 mg/kg every 3 weeks.

In an embodiment, a dosing regimen comprises administration of a B7-H3-ADC at a dose of about 2.75 mg/kg every 3 weeks and lorigerlimab at a dose of about 3 mg/kg every 3 weeks. In an embodiment, a dosing regimen comprises administration of a B7-H3-ADC at a dose of about 2.75 mg/kg every 3 weeks and lorigerlimab at a dose of about 6 mg/kg every 3 weeks.

In an embodiment, a dosing regimen comprises administration of a B7-H3-ADC at a dose of about 3 mg/kg every 3 weeks and lorigerlimab at a dose of about 3 mg/kg every 3 weeks. In an embodiment, a dosing regimen comprises administration of a B7-H3-ADC at a dose of about 3 mg/kg every 3 weeks and lorigerlimab at a dose of about 6 mg/kg every 3 weeks.

In an embodiment, a dosing regimen comprises administration of a B7-H3-ADC at a dose of about 1 mg/kg to about 3 mg/kg every 4 weeks and lorigerlimab at a dose of about 1 mg/kg to about 6 mg/kg administered every 4 weeks. In an embodiment, a dosing regimen comprises administration of a B7-H3-ADC at a dose of about 1 mg/kg to about 2 mg/kg every 4 weeks and lorigerlimab at a dose of about 3 mg/kg to about 6 mg/kg administered every 4 weeks. In an embodiment, a dosing regimen comprises administration of a B7-H3-ADC at a dose of about 2 mg/kg to about 3 mg/kg every 4 weeks and lorigerlimab at a dose of about 3 mg/kg to about 6 mg/kg administered every 4 weeks. In an embodiment, a dosing regimen comprises administration of a B7-H3-ADC at a dose of about 1 mg/kg every 4 weeks and lorigerlimab at a dose of about 3 mg/kg to about 6 mg/kg every 4 weeks. In an embodiment, a dosing regimen comprises administration of a B7-H3-ADC at a dose of about 1.25 mg/kg every 4 weeks and lorigerlimab at a dose of about 3 mg/kg to 6 mg/kg every 4 weeks. In an embodiment, a dosing regimen comprises administration of a B7-H3-ADC at a dose of about 1.5 mg/kg every 4 weeks and lorigerlimab at a dose of about 3 mg/kg to 6 mg/kg every 4 weeks. In an embodiment, a dosing regimen comprises administration of a B7-H3-ADC at a dose of about 1.75 mg/kg every 4 weeks and lorigerlimab at a dose of about 3 mg/kg to 6 mg/kg every 4 weeks. In an embodiment, a dosing regimen comprises administration of a B7-H3-ADC at a dose of about 2 mg/kg every 4 weeks and lorigerlimab at a dose of about 3 mg/kg to 6 mg/kg every 4 weeks. In an embodiment, a dosing regimen comprises administration of a B7-H3-ADC at a dose of about 2.1 mg/kg every 4 weeks and lorigerlimab at a dose of about 3 mg/kg to 6 mg/kg every 4 weeks. In an embodiment, a dosing regimen comprises administration of a B7-H3-ADC at a dose of about 2.2 mg/kg every 4 weeks and lorigerlimab at a dose of about 3 mg/kg to 6 mg/kg every 4 weeks. In an embodiment, a dosing regimen comprises administration of a B7-H3-ADC at a dose of about 2.25 mg/kg every 4 weeks and lorigerlimab at a dose of about 3 mg/kg to 6 mg/kg every 4 weeks. In an embodiment, a dosing regimen comprises administration of a B7-H3-ADC at a dose of about 2.3 mg/kg every 4 weeks and lorigerlimab at a dose of about 3 mg/kg to 6 mg/kg every 4 weeks. In an embodiment, a dosing regimen comprises administration of a B7-H3-ADC at a dose of about 2.4 mg/kg every 4 weeks and lorigerlimab at a dose of about 3 mg/kg to 6 mg/kg every 4 weeks. In an embodiment, a dosing regimen comprises administration of a B7-H3-ADC at a dose of about 2.5 mg/kg every 4 weeks and lorigerlimab at a dose of about 3 mg/kg to 6 mg/kg every 4 weeks. In an embodiment, a dosing regimen comprises administration of a B7-H3-ADC at a dose of about 2.6 mg/kg every 4 weeks and lorigerlimab at a dose of about 3 mg/kg to 6 mg/kg every 4 weeks. In an embodiment, a dosing regimen comprises administration of a B7-H3-ADC at a dose of about 2.7 mg/kg every 4 weeks and lorigerlimab at a dose of about 3 mg/kg to 6 mg/kg every 4 weeks. In an embodiment, a dosing regimen comprises administration of a B7-H3-ADC at a dose of about 2.75 mg/kg every 4 weeks and lorigerlimab at a dose of about 3 mg/kg to 6 mg/kg every 4 weeks. In an embodiment, a dosing regimen comprises administration of a B7-H3-ADC at a dose of about 3 mg/kg every 4 weeks and lorigerlimab at a dose of about 3 mg/kg to 6 mg/kg every 4 weeks.

In an embodiment, a dosing regimen comprises administration of a B7-H3-ADC at a dose of about 1 mg/kg every 4 weeks and lorigerlimab at a dose of about 3 mg/kg every 4 weeks. In an embodiment, a dosing regimen comprises administration of a B7-H3-ADC at a dose of about 1 mg/kg every 4 weeks and lorigerlimab at a dose of about 6 mg/kg every 4 weeks.

In an embodiment, a dosing regimen comprises administration of a B7-H3-ADC at a dose of about 1.25 mg/kg every 4 weeks and lorigerlimab at a dose of about 3 mg/kg every 4 weeks. In an embodiment, a dosing regimen comprises administration of a B7-H3-ADC at a dose of about 1.25 mg/kg every 4 weeks and lorigerlimab at a dose of about 6 mg/kg every 4 weeks.

In an embodiment, a dosing regimen comprises administration of a B7-H3-ADC at a dose of about 1.5 mg/kg every 4 weeks and lorigerlimab at a dose of about 3 mg/kg every 4 weeks. In an embodiment, a dosing regimen comprises administration of a B7-H3-ADC at a dose of about 1.5 mg/kg every 4 weeks and lorigerlimab at a dose of about 6 mg/kg every 4 weeks.

In an embodiment, a dosing regimen comprises administration of a B7-H3-ADC at a dose of about 1.75 mg/kg every 4 weeks and lorigerlimab at a dose of about 3 mg/kg every 4 weeks. In an embodiment, a dosing regimen comprises administration of a B7-H3-ADC at a dose of about 1.75 mg/kg every 4 weeks and lorigerlimab at a dose of about 6 mg/kg every 4 weeks.

In an embodiment, a dosing regimen comprises administration of a B7-H3-ADC at a dose of about 2 mg/kg every 4 weeks and lorigerlimab at a dose of about 3 mg/kg every 4 weeks. In an embodiment, a dosing regimen comprises administration of a B7-H3-ADC at a dose of about 2 mg/kg every 4 weeks and lorigerlimab at a dose of about 6 mg/kg every 4 weeks.

In an embodiment, a dosing regimen comprises administration of a B7-H3-ADC at a dose of about 2.1 mg/kg every 4 weeks and lorigerlimab at a dose of about 3 mg/kg every 4 weeks. In an embodiment, a dosing regimen comprises administration of a B7-H3-ADC at a dose of about 2.1 mg/kg every 4 weeks and lorigerlimab at a dose of about 6 mg/kg every 4 weeks.

In an embodiment, a dosing regimen comprises administration of a B7-H3-ADC at a dose of about 2.2 mg/kg every 4 weeks and lorigerlimab at a dose of about 3 mg/kg every 4 weeks. In an embodiment, a dosing regimen comprises administration of a B7-H3-ADC at a dose of about 2.2 mg/kg every 4 weeks and lorigerlimab at a dose of about 6 mg/kg every 4 weeks.

In an embodiment, a dosing regimen comprises administration of a B7-H3-ADC at a dose of about 2.25 mg/kg every 4 weeks and lorigerlimab at a dose of about 3 mg/kg every 4 weeks. In an embodiment, a dosing regimen comprises administration of a B7-H3-ADC at a dose of about 2.25 mg/kg every 4 weeks and lorigerlimab at a dose of about 6 mg/kg every 4 weeks.

In an embodiment, a dosing regimen comprises administration of a B7-H3-ADC at a dose of about 2.3 mg/kg every 4 weeks and lorigerlimab at a dose of about 3 mg/kg every 4 weeks. In an embodiment, a dosing regimen comprises administration of a B7-H3-ADC at a dose of about 2.3 mg/kg every 4 weeks and lorigerlimab at a dose of about 6 mg/kg every 4 weeks.

In an embodiment, a dosing regimen comprises administration of a B7-H3-ADC at a dose of about 2.4 mg/kg every 4 weeks and lorigerlimab at a dose of about 3 mg/kg every 4 weeks. In an embodiment, a dosing regimen comprises administration of a B7-H3-ADC at a dose of about 2.4 mg/kg every 4 weeks and lorigerlimab at a dose of about 6 mg/kg every 4 weeks.

In an embodiment, a dosing regimen comprises administration of a B7-H3-ADC at a dose of about 2.5 mg/kg every 4 weeks and lorigerlimab at a dose of about 3 mg/kg every 4 weeks. In an embodiment, a dosing regimen comprises administration of a B7-H3-ADC at a dose of about 2.5 mg/kg every 4 weeks and lorigerlimab at a dose of about 6 mg/kg every 4 weeks.

In an embodiment, a dosing regimen comprises administration of a B7-H3-ADC at a dose of about 2.6 mg/kg every 4 weeks and lorigerlimab at a dose of about 3 mg/kg every 4 weeks. In an embodiment, a dosing regimen comprises administration of a B7-H3-ADC at a dose of about 2.6 mg/kg every 4 weeks and lorigerlimab at a dose of about 6 mg/kg every 4 weeks.

In an embodiment, a dosing regimen comprises administration of a B7-H3-ADC at a dose of about 2.7 mg/kg every 4 weeks and lorigerlimab at a dose of about 3 mg/kg every 4 weeks. In an embodiment, a dosing regimen comprises administration of a B7-H3-ADC at a dose of about 2.7 mg/kg every 4 weeks and lorigerlimab at a dose of about 6 mg/kg every 4 weeks.

In an embodiment, a dosing regimen comprises administration of a B7-H3-ADC at a dose of about 2.75 mg/kg every 4 weeks and lorigerlimab at a dose of about 3 mg/kg every 4 weeks. In an embodiment, a dosing regimen comprises administration of a B7-H3-ADC at a dose of about 2.75 mg/kg every 4 weeks and lorigerlimab at a dose of about 6 mg/kg every 4 weeks.

In an embodiment, a dosing regimen comprises administration of a B7-H3-ADC at a dose of about 3 mg/kg every 4 weeks and lorigerlimab at a dose of about 3 mg/kg every 4 weeks. In an embodiment, a dosing regimen comprises administration of a B7-H3-ADC at a dose of about 3 mg/kg every 4 weeks and lorigerlimab at a dose of about 6 mg/kg every 4 weeks.

It is contemplated that in the above embodiments, administration occurs at the predetermined frequency or periodicity, or within 1-3 days of such scheduled dosing interval, such that administration occurs 1-3 day before, 1-3 days after, or on the day of a scheduled dose, e.g., every 3 weeks (±3 days). It is specifically contemplated that in the above embodiments, a B7-H3-ADC and a PD-1×CTLA-4 bispecific molecule are administered by IV infusion within a 24-hour period. In certain embodiments, a B7-H3-ADC and a PD-1×CTLA-4 bispecific molecule are administered by IV infusion according to any of the above dosing regimens for a duration (i.e., course of treatment) of at least 1 month or more, at least 3 months or more, or at least 6 months or more, or at least 12 months or more. A treatment duration of at least 6 months or more, or for at least 12 months or more, or until remission of disease or unmanageable toxicity is observed, is particularly contemplated. In certain embodiments, treatment continues for a period of time after remission of disease.

In certain embodiments, a B7-H3-ADC and a PD-1×CTLA-4 bispecific molecule are administered by IV infusion. The molecules are thus diluted (separately or together) into an infusion bag comprising a suitable diluent, e.g., 0.9% sodium chloride or dextrose 5% in water (D5W). Since infusion or allergic reactions may occur, premedication for the prevention of such infusion reactions is recommended and precautions for anaphylaxis should be observed during the antibody administration. In certain embodiments, the IV infusion to be administered to the subject over a period of between about 30 minutes and about 24 hours. In certain embodiments, the IV infusion is delivered over a period of about 30-45 minutes, about 30-60 minutes, about 60-75 minutes, about 30-120 minutes, or over a greater or lesser period, if the subject does not exhibit signs or symptoms of an adverse infusion reaction. In one embodiment, a B7-H3-ADC is administered by IV infusion over a period of about 60-75 minutes. In one embodiment, a B7-H3-ADC is administered by IV infusion over a period of about 60 minutes. In one embodiment, a B7-H3-ADC is administered by IV infusion over a period of about 75 minutes. In another embodiment, the PD-1×CTLA-4 bispecific molecule is administered by IV infusion over a period of about 30-45 minutes. In another embodiment, lorigerlimab is administered by IV infusion over a period of about 30 minutes. In another embodiment, lorigerlimab is administered by IV infusion over a period of about 45 minutes.

In any of the above embodiments, a B7-H3-ADC and a PD-1×CTLA-4 bispecific molecule are administered by IV infusion concurrently, sequentially, in an alternating manner, or at different times. In certain embodiments, a B7-H3-ADC is administered before the administration of a PD-1×CTLA-4 bispecific molecule on days when both a B7-H3-ADC and a PD-1×CTLA-4 bispecific molecule are administered. In certain embodiments, a PD-1×CTLA-4 bispecific molecule is administered before the administration of a B7-H3-ADC on days when both a B7-H3-ADC and a PD-1×CTLA-4 bispecific molecule are administered. In an embodiment, a B7-H3-ADC is administered before the administration of a PD-1×CTLA-4 bispecific molecule on days when both a B7-H3-ADC and a PD-1×CTLA-4 bispecific molecule are administered. In an embodiment, a PD-1×CTLA-4 bispecific molecule is administered at least about 15-60 minutes after the administration of a B7-H3-ADC. In an embodiment, a PD-1×CTLA-4 bispecific molecule is administered at least about 15-30 minutes after the administration of a B7-H3-ADC. In an embodiment, a PD-1×CTLA-4 bispecific molecule is administered at least about 15 minutes after the administration of a B7-H3-ADC. In an embodiment, a PD-1×CTLA-4 bispecific molecule is administered at least about 30 minutes after the administration of a B7-H3-ADC.

### XI. Embodiments of the Disclosure

The disclosure concerns in part the following non-limiting embodiments (E1-E74):
E1. A method of treating a cancer in a subject in need thereof, comprising administering an anti-B7-H3 antibody-drug conjugate (B7-H3-ADC) and a PD-1×CTLA-4 bispecific molecule to said subject, wherein said B7-H3-ADC comprises the formula:

Ab-(LM)ₘ-(D)ₙ,
wherein:
Ab is a humanized B7-H3 antibody or B7-H3 binding fragment thereof that binds to B7-H3 and comprises:
(i) the CDRL1 sequence RASESIYSYLA (SEQ ID NO:22), the CDRL2 sequence NTKTLPE (SEQ ID NO:23) and the CDRL3 sequence QHHYGTPPWT (SEQ ID NO:24) in its Variable Light Chain (VL) domain, and
(ii) the CDRH1 sequence SYGMS (SEQ ID NO:25), the CDRH2 sequence TINSGGSNTYY PDSLKG (SEQ ID NO:26) and the CDRH3 sequence HDGGAMDY (SEQ ID NO:27) in its Variable Heavy Chain (VH) domain;
D is a cytotoxic duocarmycin moiety;
LM comprises at least one bond or a Linker Molecule that covalently links Ab and D;
m is an integer between 0 and n and denotes the number of bonds or Linker Molecules of said B7-H3-ADC, except when LM is a bond, m is not 0;
and
n is an integer between 1 and 10 and denotes the number of cytotoxic duocarmycin moieties covalently linked to said B7-H3-ADC molecule.
E2. The method of E1, wherein said Ab comprises:
   (i) a humanized Variable Light Chain (VL) domain comprising the amino acid sequence of SEQ ID NO:17; and
   (ii) a humanized Variable Heavy Chain (VH) domain comprising the amino acid sequence of SEQ ID NO:18.
E3. The method of any one of E1 or E2, wherein said Ab further comprises an Fc of a human IgG1, IgG2, IgG3, or IgG4.
E4. The method of E3, wherein said Fc Domain is a variant Fc Domain that comprises:
   (a) one or more amino acid modifications that reduces the affinity of the variant Fc Domain for an FcγR; and/or
   (b) one or more amino acid modifications that enhances the serum half-life of the variant Fc Domain.
E5. The method of any one of E1-E4, wherein at least one of said LM is a Linker Molecule.
E6. The method of any one of E1-E5, wherein said LM Linker Molecule is a peptidic linker.
E7. The method of E6, wherein said peptidic linker is a valine-citrulline dipeptide linker.
E8. The method of any one of E1-E7, wherein said LM Linker Molecule further comprises a self-eliminating spacer between the cleavable linker and D.
E9. The method of E8, wherein said self-eliminating spacer comprises a para-aminobenzyloxycarbonyl moiety.
E10. The method of any one of E1-E9, wherein said LM Linker Molecule further comprises a maleimide linker moiety between the cleavable linker and Ab.
E11. The method of any one of E1-E10, wherein LM is represented by the formula:

[V—(W)ₖ—(X)₁-A]
whereby said B7-H3-ADC is represented by the formula:

Ab-[V—(W)ₖ—(X)₁-A]-D
wherein:
V is a cleavable linker,
(W)ₖ—(X)₁-A is an elongated, self-eliminating spacer system, that self-eliminates via a 1,(4+2n)-elimination,
W and X are each a 1,(4+2n) electronic cascade spacer, being the same or different,
A is either a spacer group of formula (Y)ₘ, wherein Y is a 1,(4+2n) electronic cascade spacer, or a group of formula U, being a cyclisation elimination spacer,
k, l and m are independently an integer of 0 (included) to 5 (included),
n is an integer of 0 (included) to 10 (included),
with the provisos that:
   when A is (Y)ₘ: then k+l+m≥1, and
   if k+l+m=l, then n>l;
   when A is U: then k+1≥1.
W, X, and Y are independently selected from compounds having the formula:

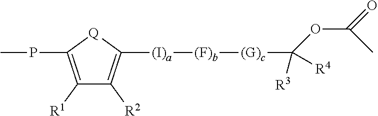

or the formula:

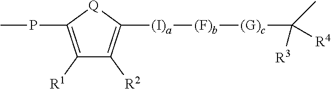

wherein: Q is —R⁵C=CR⁶—, S, O, NR⁵, —R⁵C═N—, or —N═CR⁵—
   P is NR⁷, O or S
   a, b, and c are independently an integer of 0 (included) to 5 (included);
   I, F and G are independently selected from compounds having the formula:

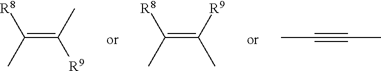

wherein R¹, R², R³, R⁴, R⁵, R⁶, R⁷, R⁸, and R⁹ independently represent H, C₁₋₆ alkyl, C₃₋₂₀ heterocyclyl, C₅₋₂₀ aryl, C₁₋₆ alkoxy, hydroxy (OH), amino (NH₂), mono-substituted amino (NRₓH), di-substituted amino (NRₓ¹Rₓ²), nitro (NO₂), halogen, CF₃, CN, CONH₂, SO₂Me, CONHMe, cyclic C₁₋₅ alkylamino, imidazolyl, C₁₋₆ alkylpiperazinyl, morpholino, thiol (SH), thioether (SRₓ), tetrazole, carboxy (COOH), carboxylate (COORₓ), sulphoxy (S(═O)₂OH), sulphonate (S(═O)₂ORₓ), sulphonyl (S(═O)₂Rₓ), sulphixy (S(═O)OH), sulphinate (S(═O)ORₓ), sulphinyl (S(═O)Rₓ), phosphonooxy (OP(═O)(OH)₂), and phosphate (OP(═O)(ORₓ)₂), where Rₓ, Rₓ¹ and Rₓ² are independently selected from a C₁₋₆ alkyl group, a C₃₋₂₀ heterocyclyl group or a C₅₋₂₀ aryl group, two or more of the substituents R¹, R², R³, R⁴, R⁵, R⁶, R⁷, R⁸, or R⁹ optionally being connected to one another to form one or more aliphatic or aromatic cyclic structures;
U is selected from compounds having the formula:

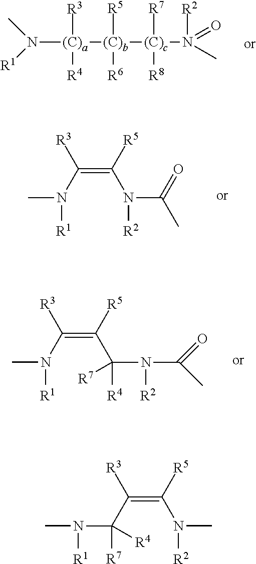

wherein:
a, b and c are independently selected to be an integer of 0 or 1;
   provided that a+b+c=2 or 3;
R¹ and/or R² independently represent H, C₁₋₆ alkyl, the alkyl being optionally substituted with one or more of the following groups: hydroxy (OH), ether (ORₓ), amino (NH₂), mono-substituted amino (NRₓH), disubstituted amino (NRₓ¹Rₓ²), nitro (NO₂), halogen, CF₃, CN, CONH₂, SO₂Me, CONHMe, cyclic C₁₋₈ alkylamino, imidazolyl, C₁₋₆ alkylpiperazinyl, morpholino, thiol (SH), thioether (SRₓ), tetrazole, carboxy (COOH), carboxylate (COORₓ), sulphoxy (S(═O)₂OH), sulphonate (S(═O)₂₀Rₓ), sulphonyl (S(═O)₂Rₓ), sulphixy (S(═O)OH), sulphinate (S(═O)ORₓ), sulphinyl (S(═O)Rₓ), phosphonooxy (OP(═O)(OH)₂), and phosphate (OP(═O)(ORₓ)₂), where Rₓ, Rₓ¹ and Rₓ² are selected from a C₁₋₆ alkyl group, a C₃₋₂₀ heterocyclyl group or a C₅₋₂₀ aryl group; and
R³, R⁴, R⁵, R⁶, R⁷ and R⁸ independently represent H, C₁₋₆ alkyl, C₃₋₂₀ heterocyclyl, C₅₋₂₀ aryl, C₁₋₆ alkoxy, hydroxy (OH), amino (NH₂), mono-substituted amino (NRₓH), disubstituted amino (NRₓ¹Rₓ²), nitro (NO₂), halogen, CF₃, CN, CONH₂, SO₂Me, CONHMe, cyclic C₁₋₈ alkylamino, imidazolyl, C₁₋₆ alkylpiperazinyl, morpholino, thiol (SH), thioether (SRₓ), tetrazole, carboxy (COOH), carboxylate (COORₓ), sulphoxy (S(═O)₂OH), sulphonate (S(═O)₂₀Rₓ), sulphonyl (S(═O)₂Rₓ), sulphixy (S(═O)OH), sulphinate (S(═O)ORₓ), sulphinyl (S(═O)Rₓ), phosphonooxy (OP(═O)(OH)₂), and phosphate (OP(═O)(ORₓ)₂), where Rₓ, Rₓ¹ and Rₓ² are selected from a C₁₋₆ alkyl group, a C₃₋₂₀ heterocyclyl group or a C₅₋₂₀ aryl group, and two or more of the substituents R¹, R², R³, R⁴, R⁵, R⁶, R⁷, or R⁸ are optionally connected to one another to form one or more aliphatic or aromatic cyclic structures.
E12. The method of E11, wherein said LM linker molecule comprises:
   (1) p-aminobenzyloxycarbonyl-p-aminobenzyloxycarbonyl;
   (2) p-aminobenzyloxycarbonyl-p-aminobenzyloxycarbonyl-p-aminobenzyloxycarbonyl;
   (3) p-ammocinnamyloxycarbonyl;
   (4) p-aminocinnamyloxycarbonyl-p-aminobenzyloxycarbonyl;
   (5) p-amino-benzyloxycarbonyl-p-aminocinnamyloxycarbonyl;
   (6) p-aminocinnamyloxycarbonyl-p-aminocinnamyloxycarbonyl;
   (7) p-aminophenylpentadienyloxycarbonyl;
   (8) p-aminophenylpentadienyloxycarbonyl-p-aminocinnamyloxycarbonyl;
   (9) p-aminophenylpentadienyloxycarbonyl-p-aminobenzyloxycarbonyl;
   (10) p-aminophenylpentadienyloxycarbonyl-p-aminophenylpentadienyloxycarbonyl;
   (11) p-aminobenzyloxycarbonyl(methylamino)ethyl(methylamino) carbonyl;
   (12) p-aminocinnamyloxycarbonyl(methylamino)ethyl(methylamino) carbonyl;
   (13) p-aminobenzyloxycarbonyl-p-aminobenzyloxycarbonyl(methylamino) ethyl(methylamino)carbonyl;
   (14) p-aminocinnamyloxycarbonyl-p-aminobenzyloxycarbonyl (methylamino)ethyl(methylamino)carbonyl;
   (15) p-aminobenzyloxycarbonyl-p-aminocinnamyloxycarbonyl (methylamino)ethyl(methylamino)-carbonyl;
   (16) p-aminocinnamyloxycarbonyl-p-aminocinnamyloxycarbonyl (methylamino)ethyl(methylamino)carbonyl;
   (17) p-aminobenzyloxycarbonyl-p-aminobenzyl;
   (18) p-aminobenzyloxycarbonyl-p-aminobenzyloxycarbonyl-p-aminobenzyl;
   (19) p-aminocinnamyl;
   (20) p-aminocinnamyloxycarbonyl-p-aminobenzyl;
   (21) p-aminobenzyloxycarbonyl-p-aminocinnamyl;
   (22) p-amino-cinnamyloxycarbonyl-p-aminocinnamyl;
   (23) p-aminophenylpentadienyl;
   (24) p-aminophenylpentadienyloxycarbonyl-p-aminocinnamyl;
   (25) p-aminophenylpentadienyloxycarbonyl-p-aminobenzyl; or
   (26) p-aminophenylpentadienyloxycarbonyl-p-aminophenylpentadienyl.
E13. The method of any one of E1-E12, wherein said LM Linker Molecule is conjugated to the side chain of an amino acid of a polypeptide chain of the Ab and binds the Ab to a molecule of the cytotoxic duocarmycin moiety D.
E14. The method of any one of E1-E13, wherein said cytotoxic duocarmycin moiety D comprises a duocarmycin cytotoxin selected from the group consisting of: duocarmycin A, duocarmycin B1, duocarmycin B2, duocarmycin C1, duocarmycin C2, duocarmycin D, duocarmycin SA, CC-1065, adozelesin, bizelesin, carzelesin (U-80244), seco-duocarmycin (seco-DUBA) and spiro-duocarmycin (spiro-DUBA).
E15. The method of E14, wherein the cytotoxic duocarmycin moiety D comprises seco-DUBA.
E16. The method of any of E1-E15, wherein said LM Linker Molecule is covalently linked to the Ab via reduced inter-chain disulfides.
E17. The method of any one of E1-E16, wherein said Ab comprises:
   (i) a light chain comprising the amino acid sequence of SEQ ID NO:19; and
   (ii) a heavy chain comprising the amino acid sequence of SEQ ID NO:20;
   said D comprises seco-DUBA; and
   said LM comprises a Linker Molecule comprising a maleimide linker moiety, a valine-citrulline dipeptide linker, and a para-aminobenzyloxycarbonyl moiety.
E18. The method of any one of E1-E17, wherein said B7-H3-ADC is administered at a therapeutically effective or prophylactically effective dose of:
   a) about 1 mg/kg to about 3 mg/kg every 3 weeks; or
   b) about 2 mg/kg to about 3 mg/kg every 3 weeks.
E19. The method of any one of E1-E17, wherein said B7-H3-ADC is administered at a therapeutically effective or prophylactically effective dose of:
   a) about 1 mg/kg to about 3 mg/kg every 4 weeks; or
   b) about 2 mg/kg to about 3 mg/kg every 4 weeks.
E20. The method of any one of E1-E19, wherein said dose of said B7-H3-ADC is administered as a single dose.
E21. The method of any one of E1-E19, wherein said dose of said B7-H3-ADC is administered as a fractionated dose in two or more separate administrations.
E22. The method of any one of E1-E19 or E21, wherein said fractionated dose comprises two separate administrations administered within a 3-week cycle or 4-week cycle.
E23. The method of any one of E1-E22, wherein;
   (a) said B7-H3-ADC is administered before the administration of said PD-1×CTLA-4 bispecific molecule on days when both said B7-H3-ADC and said PD-1×CTLA-4 bispecific molecule are administered; or
   (b) said PD-1×CTLA-4 is administered before the administration of said B7-H3-ADC bispecific molecule on days when both said B7-H3-ADC and said PD-1×CTLA-4 bispecific molecule are administered.
E24. The method of any one of E1-E22, wherein:
   (a) said PD-1×CTLA-4 bispecific molecule is administered at least about 15-30 minutes after said B7-H3-ADC is administered; or
   (b) said B7-H3-ADC is administered at least about 15-30 minutes after said PD-1×CTLA-4 bispecific molecule is administered.
E25. The method of any one of E1-E24, wherein said PD-1×CTLA-4 bispecific molecule is selected from the group consisting of: lorigerlimab, MEDI5752, vudalimab, and cadonilimab.
E26. The method of any one of E1-E25, wherein said PD-1×CTLA-4 bispecific molecule is lorigerlimab.
E27. The method of E26, wherein said lorigerlimab is administered at a dose of about 1 mg/kg, about 3 mg/kg or about 6 mg/kg every 3 weeks.
E28. The method of E27, wherein said lorigerlimab is administered at a dose of about 6 mg/kg every 3 weeks.
E29. The method of E26, wherein said lorigerlimab is administered at a dose of about 1 mg/kg, about 3 mg/kg or about 6 mg/kg every 4 weeks.
E30. The method of E29, wherein said lorigerlimab is administered at a dose of about 6 mg/kg every 4 weeks.
E31. The method of any one of E1-E30, wherein said B7-H3-ADC is administered at a dose of about 1 mg/kg.
E32. The method of any one of E1-E30, wherein said B7-H3-ADC is administered at a dose of about 1.25 mg/kg.
E33. The method of any one of E1-E30, wherein said B7-H3-ADC is administered at a dose of about 1.5 mg/kg.
E34. The method of any one of E1-E30, wherein said B7-H3-ADC is administered at a dose of about 1.75 mg/kg.
E35. The method of any one of E1-E30, wherein said B7-H3-ADC is administered at a dose of about 2 mg/kg.
E36. The method of any one of E1-E30, wherein said B7-H3-ADC is administered at a dose of about 2.1 mg/kg.
E37. The method of any one of E1-E30, wherein said B7-H3-ADC is administered at a dose of about 2.2 mg/kg.
E38. The method of any one of E1-E30, wherein said B7-H3-ADC is administered at a dose of about 2.25 mg/kg.
E39. The method of any one of E1-E30, wherein said B7-H3-ADC is administered at a dose of about 2.3 mg/kg.
E40. The method of any one of E1-E30, wherein said B7-H3-ADC is administered at a dose of about 2.4 mg/kg.
E41. The method of any one of E1-E30, wherein said B7-H3-ADC is administered at a dose of about 2.5 mg/kg.
E42. The method of any one of E1-E30, wherein said B7-H3-ADC is administered at a dose of about 2.6 mg/kg.
E43. The method of any one of E1-E30, wherein said B7-H3-ADC is administered at a dose of about 2.7 mg/kg.
E44. The method of any one of E1-E30, wherein said B7-H3-ADC is administered at a dose of about 2.75 mg/kg.
E45. The method of any one of E1-E30, wherein said B7-H3-ADC is administered at a dose of about 3 mg/kg.
E46. The method of any one of E1-E45, wherein said B7-H3-ADC is administered by intravenous (IV) infusion.
E47. The method of E46, wherein said IV infusion of said B7-H3-ADC is over a period of at least about 60-120 minutes.
E48. The method of E47, wherein said IV infusion of said B7-H3-ADC is over a period of at least about 60 minutes.
E49. The method of any one of E1-E48, wherein said PD-1×CTLA-4 bispecific molecule is administered by IV infusion.
E50. The method of E49, wherein said IV infusion of said PD-1×CTLA-4 bispecific molecule is over a period of at least about 30-120 minutes.
E51. The method of E50, wherein said IV infusion of said PD-1×CTLA-4 bispecific molecule is over a period of at least about 30 minutes.
E52. The method of any one of E1-E51, wherein said cancer is selected from the group consisting of: an adrenal gland cancer, an AIDS-associated cancer, an alveolar soft part sarcoma, an astrocytic tumor, an anal cancer, squamous cell carcinoma of the anal canal (SCAC), a bladder cancer, a bone cancer, a brain and spinal cord cancer, a metastatic brain tumor, a B-cell cancer, a breast cancer, a HER2+ breast cancer, triple negative breast cancer (TNBC), a carotid body tumors, a cervical cancer, a chondrosarcoma, a chordoma, a chromophobe renal cell carcinoma, a clear cell carcinoma, a colon cancer, a colorectal cancer (CRC), a non-microsatellite instability high colorectal cancer (non-MSI-H CRC), a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, a gastric cancer, a gestational trophoblastic disease, a germ cell tumor, a head and neck cancer, a glioblastoma, a hematological malignancy, a hepatocellular carcinoma, an islet cell tumor, a Kaposi's Sarcoma, a kidney cancer, a leukemia, an acute myeloid leukemia, a liposarcoma/malignant lipomatous tumor, a dedifferentiated liposarcoma, a liver cancer, a lymphoma, a lung cancer, a non-small-cell lung cancer (NSCLC), a medulloblastoma, a melanoma, a cutaneous melanoma, a meningioma, a mesothelioma pharyngeal cancer, a multiple endocrine neoplasia, a multiple myeloma, a myelodysplastic syndrome, a myxofibrosarcoma, a neuroblastoma, a neuroendocrine tumors, an ovarian cancer, a pancreatic cancer, a papillary thyroid carcinoma, a parathyroid tumor, a pediatric cancer, a peripheral nerve sheath tumor, a phaeochromocytoma, a pituitary tumor, a prostate cancer, a metastatic castration resistant prostate cancer (mCRPC), a posterior uveal melanoma, a renal cell cancer, a renal cell carcinoma (RCC), a renal metastatic cancer, a rhabdoid tumor, a rhabdomyosarcoma, a sarcoma, a skin cancer, a small round blue cell tumor of childhood, a neuroblastoma, a soft tissue sarcoma, a pleomorphic undifferentiated sarcoma, a squamous cell cancer, a squamous cell cancer of the head and neck (SCCHN), a stomach cancer, a synovial sarcoma, a testicular cancer, a thymic carcinoma, a thymoma, a thyroid cancer, a thyroid metastatic cancer, and a uterine cancer.
E53. The method of E52, wherein said cancer is selected from the group consisting of anal cancer, SCAC, breast cancer, TNBC, cervical cancer, colorectal cancer, non-microsatellite instability high colorectal cancer (non-MSI-H CRC), head and neck cancer, kidney cancer, renal cell carcinoma, liver cancer, hepatocellular carcinoma, lung cancer, NSCLC, melanoma, cutaneous melanoma, posterior uveal melanoma, ovarian cancer, pancreatic cancer, prostate cancer, mCRPC, soft tissue sarcoma, dedifferentiated liposarcoma, myxofibrosarcoma, pleomorphic undifferentiated sarcoma, synovial sarcoma, squamous cell cancer, and SCCHN.
E54. The method of any one of E1-E53, wherein said cancer is prostate cancer.
E55. The method of any one of E46-E54, wherein said prostate cancer is mCRPC.
E56. The method of any one of E1-E53, wherein said cancer is liver cancer.
E57. The method of any one of E52, E53, or E56, wherein said liver cancer is hepatocellular carcinoma.
E58. The method of any one of E1-E53, wherein said cancer is kidney cancer.
E59. The method of any one of E52, E53, or E58, where said kidney cancer is renal cell carcinoma.
E60. The method of any one of E1-E53, wherein said cancer is ovarian cancer.
E61. The method of any one of E1-E53, where said cancer is pancreatic cancer.
E62. The method of any one of E1-E53, wherein said cancer is anal cancer.
E63. The method of any one of E52, E53, or E62, wherein said anal cancer is SCAC.
E64. The method of any one of E1-E53, wherein said cancer is a squamous cell cancer.
E65. The method of any one of E52, E53, or E64, wherein said squamous cell cancer is SCCHN.
E66. The method of any one of E1-E53, wherein said cancer is breast cancer.
E67. The method of any one of E52, E53, or E66, wherein said breast cancer is TNBC.
E68. The method of any one of E1-E53, wherein said cancer is melanoma.
E69. The method of any one of E52, E53, or E68, wherein said melanoma is a cutaneous melanoma or a posterior uveal melanoma.
E70. The method of any one of E1-E53, wherein said cancer is lung cancer.
E71. The method of any one of E52, E53, or E70, wherein said lung cancer is NSCLC.
E72. The method of any one of E1-E53, wherein said cancer is cervical cancer.
E73. The method of any one of E1-E53, wherein said cancer is colorectal cancer.
E74. The method of any one of E52, E53, or E73, wherein said colorectal cancer is non-MSI-H colorectal cancer.
E75. The method of any one of E1-E53, wherein said cancer is a soft tissue sarcoma.
E76. The method of any one of E52, E53, or E75, wherein said soft tissue sarcoma is dedifferentiated liposarcoma, myxofibrosarcoma, pleomorphic undifferentiated sarcoma, or synovial sarcoma.
E77. The method of any one of E1-E76, where said cancer expresses B7-H3.
E78. The method of any one of E1-E77, further comprising administering a therapeutically or prophylactically effective amount of one or more additional therapeutic agents or chemotherapeutic agents.
E79. The method of E78, wherein said chemotherapeutic agent is a platinum-based chemotherapeutic agent.
E80. The method of E79, wherein said chemotherapeutic agent is a taxane.
E81. The method of any one of E1-E80, wherein said subject in need thereof is a human.

### EXAMPLES

Having now generally described the invention, the same will be more readily understood through reference to the following Examples. The following examples illustrate various methods for compositions in the diagnostic or treatment methods of the invention. The examples are intended to illustrate, but in no way limit, the scope of the invention.

### Example 1

### In Vivo Efficacy of Single or Fractionated Dose Administration of MGC018 in CD-1 Nude Mice

While MGC018 was utilized in the animal studies presented herein, it will be understood in view of the teachings herein that similar studies may be designed using any B7-H3-ADC described herein.

To demonstrate anti-tumor activity of MGC018 in a fractionated/repeat-dose study, MGC018 was evaluated for in vivo toxicity in an immunodeficient CD-1 Nude (homozygous) mouse model using a human Calu-6 lung cancer cell line derived xenograft tumor model. In brief, ˜5×10⁶ tumor cells (suspended in 1:1 media and MATRIGEL®) were subcutaneously implanted into the flank of female CD-1 Nude mice (homozygous; Charles River Laboratories). When tumors had reached a volume of approximately 100-235 mm3 (average 172 mm³) on day 21, the mice were randomized and MGC018 or control vehicle were administered intravenously. In these studies, mice were administered either a single dose (12 mg/kg, QW×1) of MGC018 or a fractionated dose of MGC018 (0.3 mg/kg, 1 mg/kg, or 3 mg/kg) once a week for 4 weeks (QW×4W). Vehicle was administered as a control on day 21. Tumors were measured twice weekly by orthogonal measurements with electronic calipers, with tumor volumes calculated as: (length×width×height)/2. The tumor volume (relative to control) was determined (“T/C”). A finding that the tumor volume of treated animals had decreased to ≤5 mm³ during the study period was considered to denote a Complete Regression (“CR”) and a Partial Regression (“PR”) was defined when tumors were reduced by 50% or greater from day of dosing at any point during the study. Anti-tumor Activity was evaluated according to National Cancer Institute (NCI) standards; a T/C≤42% is the minimum level of anti-tumor activity, while a T/C value of >42% is inactive. A T/C<10% is considered highly active.

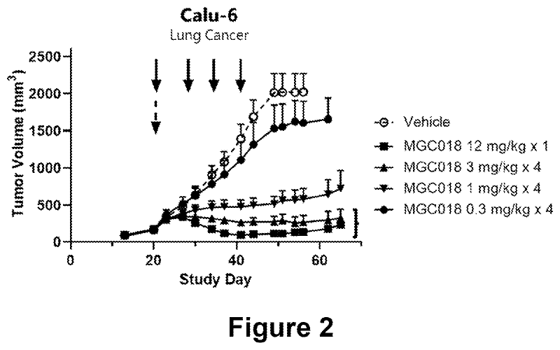

The results of this study with respect to subcutaneously implanted Calu-6 cells are presented in Table 1 and in FIG. 2. The results of this study show that a fractionated dose of MGC018 at all concentrations tested (0.3 mg/kg, 1 mg/kg and 3 mg/kg; QW×4W) yields near equivalent anti-tumor activity against Calu-6 cells as a single dose of MGC018 (12 mg/kg, QW×1).

TABLE 1
Anti-Tumor Activity of MGC018 Against
Calu-6 Cells in a CD-1 Nude Mouse Model

| | | | | | | Anti- |
|---|---|---|---|---|---|---|
| | Dose | Administration | % T/C at | | | Tumor |
| Treatment | (mg/kg) | Schedule | Day 44 | PR | CR | Activity |
| MGC018 | 12 | QW × 1 | 6 | 2/7 | 0/7 | Highly |
| | | | | | | Active |
| MGC018 | 3 | QW × 4W | 17 | 2/7 | 0/7 | Active |
| MGC018 | 1 | QW × 4W | 30 | 0/7 | 0/7 | Active |
| MGC018 | 0.3 | QW × 4W | 78 | 0/7 | 0/7 | Active |

Abbreviations in Table 1: QW: single dose on the first week; QW × 4W: weekly dose every week for 4 weeks; T/C: tumor volume relative to control; PR: partial regression; CR: complete regression.

### Example 2

### In Vivo Efficacy of Single or Fractionated Dose Administration of MGC018 in CES1c Knockout Mice

To further demonstrate anti-tumor activity of MGC018 in a fractionated/repeat-dose study, MGC018 was evaluated for in vivo toxicity in an immunodeficient SCID CES1c-KO mouse model using human A375.S2 melanoma cell line, human Calu-6 lung cancer cell line and human MDA-MB-468 triple negative breast cancer cell line derived xenograft tumor models.

### 2.1 A375.S2 Melanoma Cell Study

In brief, ˜5×10⁶ A375.S2 tumor cells (suspended in 1:1 media and MATRIGEL®) were subcutaneously implanted into the flank of female SCID CES1c-KO mice (Charles River Laboratories). When tumors had reached a volume of approximately 70-255 mm³ (average 124 mm³) on day 22, the mice were randomized and MGC018 or control vehicle were administered intravenously. In these studies, mice were administered either a single dose (1 mg/kg or 3 mg/kg, QW×1) of MGC018 or a fractionated dose of MGC018 (0.3 mg/kg or 1 mg/kg) once a week for 4 weeks (QW×4W). Vehicle was administered as a control on day 22. Tumors were measured twice weekly by orthogonal measurements with electronic calipers, with tumor volumes calculated as: (length×width×height)/2. The tumor volume (relative to control) was determined (“T/C”). A finding that the tumor volume of treated animals had decreased to ≤5 mm³ during the study period was considered to denote a Complete Regression (“CR”) and a Partial Regression (“PR”) was defined when tumors were reduced by 50% or greater from day of dosing at any point during the study. Anti-tumor Activity was evaluated according to National Cancer Institute (NCI) standards; a T/C≤42% is the minimum level of anti-tumor activity, while a T/C value of >42% is inactive. A T/C<10% is considered highly active.

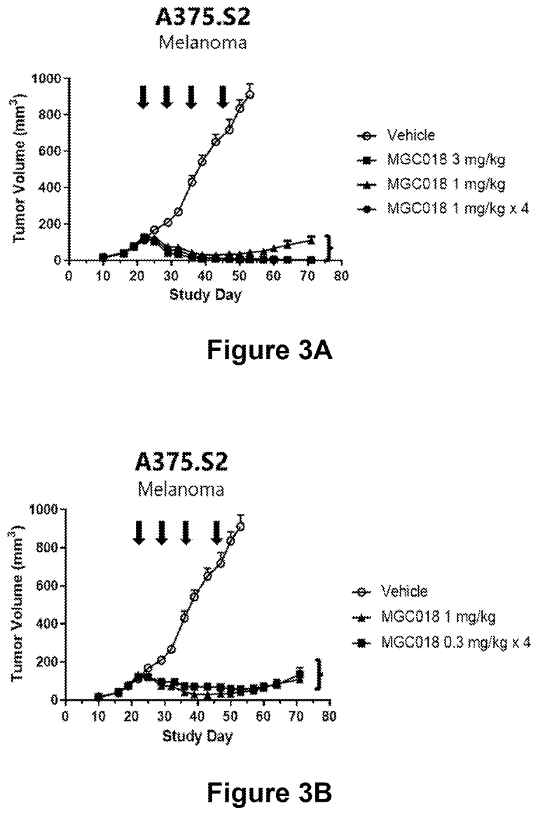

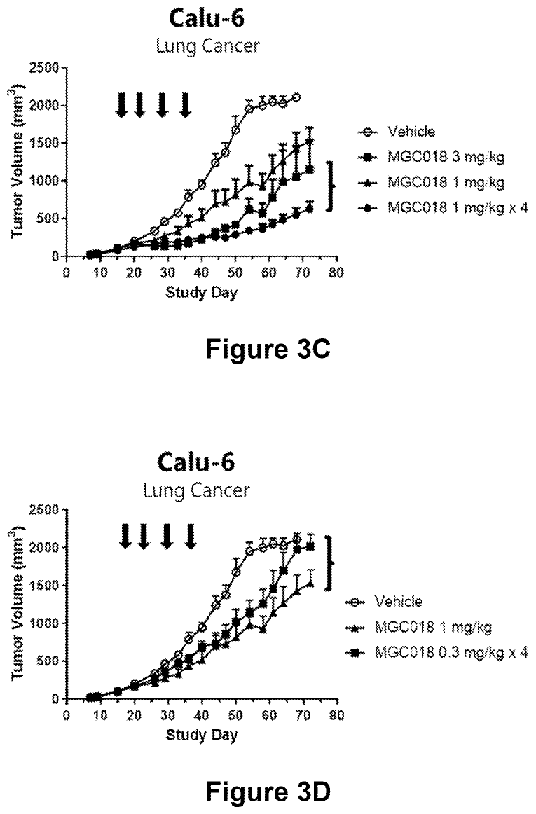

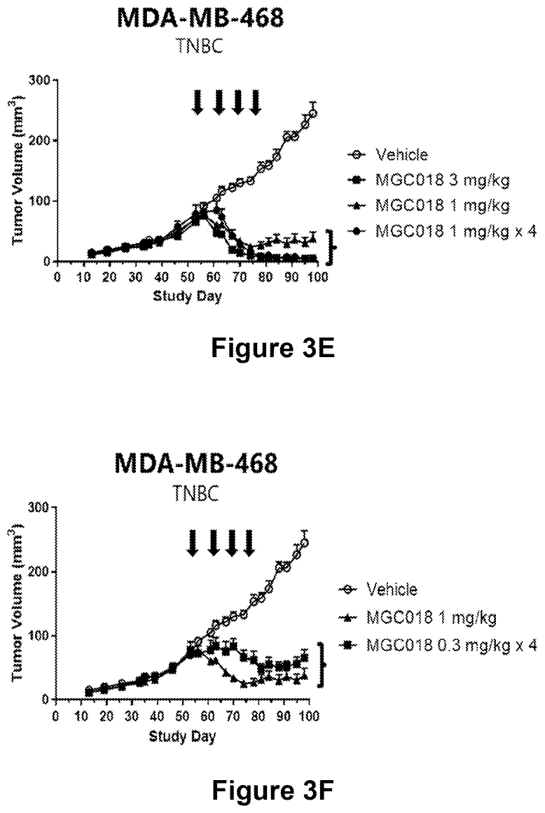

The results of this study with respect to subcutaneously implanted A375.S2 cells are presented in Table 2 and in FIGS. 3A-3B. The results of this study show that a fractionated dose of MGC018 at both concentrations tested (0.3 mg/kg and 1 mg/kg; QW×4W) yields near equivalent anti-tumor activity against A375.S2 cells as a single dose of MGC018 (1 mg/kg or 3 mg/kg, QW×1).

TABLE 2
Anti-Tumor Activity of MGC018 Against A375.S2
Cells in a SCID CES1c-KO Mouse Model

| | | | | | | Anti- |
|---|---|---|---|---|---|---|
| | Dose | Administration | % T/C at | | | Tumor |
| Treatment | (mg/kg) | Schedule | Day 53 | PR | CR | Activity |
| MGC018 | 3 | QW × 1 | 1 | 7/7 | 7/7 | Highly |
| | | | | | | Active |
| MGC018 | 1 | QW × 1 | 5 | 7/7 | 0/7 | Highly |
| | | | | | | Active |
| MGC018 | 1 | QW × 4W | 1 | 7/7 | 6/7 | Highly |
| | | | | | | Active |
| MGC018 | 0.3 | QW × 4W | 6 | 4/7 | 0/7 | Highly |
| | | | | | | Active |

Abbreviations in Table 2: QW: single dose; QW × 4W: weekly dose every week for 4 weeks; T/C: tumor volume relative to control; PR: partial regression; CR: complete regression.

### 2.2 Calu-6 Lung Cancer Cell Study

In brief, ˜5×10⁶ tumor cells (suspended in 1:1 media and MATRIGEL®) were subcutaneously implanted into the flank of female SCID CES1c-KO mice (Charles River Laboratories). When tumors had reached a volume of approximately 50-180 mm³ (average 105 mm³) on day 16, the mice were randomized and MGC018 or control vehicle were administered intravenously. In these studies, mice were administered either a single dose (1 mg/kg or 3 mg/kg, QW×1) of MGC018 or a fractionated dose of MGC018 (0.3 mg/kg or 1 mg/kg) once a week for 4 weeks (QW×4W). Vehicle was administered as a control on day 16. Tumors were measured twice weekly by orthogonal measurements with electronic calipers, with tumor volumes calculated as: (length×width×height)/2. The tumor volume (relative to control) was determined (“T/C”). A finding that the tumor volume of treated animals had decreased to ≤5 mm³ during the study period was considered to denote a Complete Regression (“CR”) and a Partial Regression (“PR”) was defined when tumors were reduced by 50% or greater from day of dosing at any point during the study. Anti-tumor Activity was evaluated according to National Cancer Institute (NCI) standards; a T/C≤42% is the minimum level of anti-tumor activity, while a T/C value of >42% is inactive. A T/C<10% is considered highly active.

The results of this study with respect to subcutaneously implanted Calu-6 cells are presented in Table 3 and in FIGS. 3C-3D. The results of this study show that a fractionated dose of MGC018 at both concentrations tested (0.3 mg/kg and 1 mg/kg; QW×4W) yields near equivalent anti-tumor activity against Calu-6 cells as a single dose of MGC018 (1 mg/kg or 3 mg/kg, QW×1).

TABLE 3
Anti-Tumor Activity of MGC018 Against Calu-6
Cells in a SCID CES1c-KO Mouse Model

| | | | | | | Anti- |
|---|---|---|---|---|---|---|
| | Dose | Administration | % T/C at | | | Tumor |
| Treatment | (mg/kg) | Schedule | Day 50 | PR | CR | Activity |
| MGC018 | 3 | QW × 1 | 25 | 0/6 | 0/6 | Active |
| MGC018 | 1 | QW × 1 | 49 | 0/6 | 0/6 | Inactive |
| MGC018 | 1 | QW × 4W | 17 | 0/6 | 0/6 | Active |
| MGC018 | 0.3 | QW × 4W | 61 | 0/6 | 0/6 | Inactive |

Abbreviations in Table 3: QW: single dose; QW × 4W: weekly dose every week for 4 weeks; T/C: tumor volume relative to control; PR: partial regression; CR: complete regression.

### 2.3 MDA-MB-468 Triple Negative Breast Cancer Cell Study

In brief, ˜5×10⁶ tumor cells (suspended in 1:1 media and MATRIGEL®) were subcutaneously implanted into the flank of female SCID CES1c-KO mice (Charles River Laboratories). When tumors had reached a volume of approximately 45-135 mm³ (average 73 mm³) on day 54, the mice were randomized and MGC018 or control vehicle were administered intravenously. In these studies, mice were administered either a single dose (1 mg/kg or 3 mg/kg, QW×1) of MGC018 or a fractionated dose of MGC018 (0.3 mg/kg or 1 mg/kg) once a week for 4 weeks (QW×4W). Vehicle was administered as a control on day 54. Tumors were measured twice weekly by orthogonal measurements with electronic calipers, with tumor volumes calculated as: (length×width×height)/2. The tumor volume (relative to control) was determined (“T/C”). A finding that the tumor volume of treated animals had decreased to ≤5 mm³ during the study period was considered to denote a Complete Regression (“CR”) and a Partial Regression (“PR”) was defined when tumors were reduced by 50% or greater from day of dosing at any point during the study. Anti-tumor Activity was evaluated according to National Cancer Institute (NCI) standards; a T/C≤42% is the minimum level of anti-tumor activity, while a T/C value of >42% is inactive. A T/C<10% is considered highly active.

The results of this study with respect to subcutaneously implanted MDA-MB-468 cells are presented in Table 4 and in FIGS. 3E-3F. The results of this study show that a fractionated dose of MGC018 at both concentrations tested (0.3 mg/kg and 1 mg/kg; QW×4W) yields near equivalent anti-tumor activity against MDA-MB-468 cells as a single dose of MGC018 (1 mg/kg or 3 mg/kg, QW×1).

TABLE 4
Anti-Tumor Activity of MGC018 Against MDA-
MB-468 Cells in a SCID CES1c-KO Mouse Model

| | | | | | | Anti- |
|---|---|---|---|---|---|---|
| | Dose | Administration | % T/C at | | | Tumor |
| Treatment | (mg/kg) | Schedule | Day 70 | PR | CR | Activity |
| MGC018 | 3 | QW × 1 | 12 | 5/5 | 5/5 | Active |
| MGC018 | 1 | QW × 1 | 26 | 4/5 | 0/5 | Active |
| MGC018 | 1 | QW × 4W | 22 | 5/5 | 3/5 | Active |
| MGC018 | 0.3 | QW × 4W | 64 | 2/5 | 0/5 | Inactive |

Abbreviations in Table 4: QW: single dose; QW × 4W: weekly dose every week for 4 weeks; T/C: tumor volume relative to control; PR: partial regression; CR: complete regression.

### Example 3

### Predicted Pharmacokinetics for Single and Fractionated Dose Administration of MGC018 in Human Subjects

To analyze the pharmacokinetics of single and fractionated dose administration of MGC018, population pharmacokinetic (PPK) modeling was performed on clinical PK data from a dose escalation trial of MGC018. The initial model was based on data from 52 patients (516 ADC observations, 495 payload observations) and later updated with data from a total of 122 patients. MGC018 concentration values below quantification limit (BQL) were excluded from the analysis but were retained (commented out) in the data set.

The PPK analysis was conducted via nonlinear mixed-effects modeling with INTERACTION option (FOCEI) method using the NONMEM software, Version 7.5.0 (ICON Development Solutions). A quasi-steady-state (QSS) approximation of the target-mediated drug disposition (TMDD) model was used to describe the observed ADC data. All clearance and volume parameters were scaled allometrically. Goodness-of-fit plots and diagnostics (including standard error of the estimates) were used to evaluate the model fit. Due to the small sample size (52 subjects) no formal covariate analysis was attempted, although dependence of model parameters on weight, dose, and sex was investigated using diagnostic plots or by inclusion in the model. Ability of the model to describe strong covariate relationships identified by the model was tested by stratifying the diagnostic plots and model evaluation procedures by the covariates of interest. Specifically, the diagnostics and the predictive check procedures were stratified by dose (cohort).

### 3.1 Results

Of the models evaluated, a linear 2-compartment with individual random effects on clearance (CL), central volume (Vc), peripheral volume (VP), inter-compartment clearance (Q), and proportional residual variability) best described the observed ADC data and advanced for further development of the combined ADC-free payload model. The free payload was described by a 1-compartment model with random effects on free payload clearance (CL_{TOX}) and volume (V_{TOX}) parameters with the input (from ADC) to both the central and delay compartments, with the random effects on the delay rate constant Kₜᵣ, CL_{TOX}, and V_{TOX}. The final model (Model 151) was selected based on diagnostics and goodness-of-fit and used to estimate MGC018 PK parameters. According to the model, elimination from the ADC was directed to the free payload, to the delay and central free payload compartments. Approximately equal fractions of ADC elimination went to each of these compartments. The delay rate constant was estimated as 0.128 1/day (corresponding to half-life of 5.4 days) while the free payload elimination rate constant K_{TOX}=CL_{TOX}/V_{TOX} was estimated as K_{TOX}=52.5 1/day (corresponding to a half-life of about 20 minutes). Thus, the free payload kinetics were defined by the kinetics of ADC and delay between ADC elimination and input to the free payload central compartment

For a typical 75 kg subject, clearance, central volume, inter-compartment clearance and peripheral volume were estimated as CL=1.68 L/day, V=3.72 L, Q=0.0549 L/day, and Vₚ=0.700 L, respectively. Thus, terminal half-life and distribution half-life were estimated at t_{1/2}^{term}=9.18 days and t_{1/2}^{dist}=1.48 days, respectively. Among model parameters, only ADC central and peripheral volumes increased with weight (with power coefficient of 0.41), but this dependence was poorly estimated (RSE of 41%).

Model-predicted MGC018 exposure parameters are summarized in Table 5 and are based on simulated concentration-time courses over a range of doses including the fractionated dose regimen, 1 mg/kg every week for 3 weeks (QW for 3W), and 1 mg/kg on weeks 1 and 2 with no administration on weeks 3 and 4 (2Q4W). The ADC AUC and Cₘₐₓ values increased approximately dose-proportionally for doses above 1 mg/kg.

TABLE 5
Model Predicted MGC018 Exposure Parameters

| | Mean (SD) | | | Median (Range) | | |
|---|---|---|---|---|---|---|
| Dose | AUC | Cₘₐₓ | C_{trough} | AUC | Cₘₐₓ | C_{trough} |
| (mg/kg) | (μg/mL*day) | (μg/mL) | (μg/mL) | (ng/mL*day) | (ng/mL) | (ng/mL) |
| 3 mg/kg | 153 | 61.3 | 0.172 | 145 | 60.7 | 0.117 |
| Q3W | (47.3) | (11.7) | (0.21) | (23.1- | (34.4- | (0.00218- |
| | | | | 298) | 96.3) | 2.02) |
| 2 mg/kg | 102 | 40.9 | 0.06 | 96.8 | 40.5 | 0.0432 |
| Q4W | (32) | (7.8) | (0.068) | (15.4- | (23- | (0.000911- |
| | | | | 205) | 64.2) | 0.66) |
| 1 mg/kg | 152 | 21.9 | 0.259 | 144 | 21.8 | 0.169 |
| QW for | (46.9) | (4.28) | (0.299) | (23.1- | (11.9- | (0.00234- |
| 3W | | | | 294) | 35.1) | 2.7) |
| 2 mg/kg | 102 | 21.7 | 0.0873 | 96.6 | 21.7 | 0.0602 |
| 2Q4W | (31.8) | (4.22) | (0.104) | (15.4- | (11.9- | (0.00118- |
| | | | | 202) | 34.7) | 1) |

Abbreviations in Table 5: Q3W: once every 3 weeks; Q4W: once every 4 weeks; QW for 3W: once a week for 3 weeks of a 4-week cycle, with no administration on week 4; 2Q4W: 1 mg/kg administered on weeks 1 and 2 of a 4-week cycle, with no administration on weeks 3 and 4.

In summary, as reflected in Table 5, the fractionated dosing schedule of 1 mg/kg administered once weekly for 3 weeks in a 28-day (4-week) cycle has a lower Cₘₐₓ but comparable AUC relative to 3 mg/kg administered once every 3 weeks. Additionally, the fractionated dosing schedule of 1 mg/kg administered once weekly on weeks 1 and 2 of a 4-week cycle has a lower Cₘₐₓ but comparable AUC relative to 2 mg/kg administered once every 4 weeks. In summary, the fractionated dose regimens, QW for 3W and 2Q4W, are suitable alternatives to single dose administration of 3 mg/kg Q3W or 2 mg/kg Q4W, respectively, of MGC018.

### Example 4

### Dose Escalation Study of a Combination of a MGC018 and Lorigerlimab

In order to determine the tolerability of patients to a B7-H3-ADC in combination with lorigerlimab, a Phase I clinical study will be conducted. While the following protocol details the use of MGC018 in combination with lorigerlimab, it will be understood in view of the teachings herein that similar combination protocols may be designed using any of the B7-H3-ADC and any of the PD-1×CTLA-4 bispecific molecules described herein.

A dose escalation study is performed to determine the Maximum Tolerated Dose (MTD) or Maximum Administered Dose (MAD) (if no MTD is defined) of escalating doses of MGC018 administered in combination with a 6 mg/kg dose of lorigerlimab. Dose escalation will follow a conventional 3+3 design: successive cohorts of 3-6 participants each will be evaluated in sequential escalating dose cohorts (Table 6).

TABLE 6
Phase I Dose Escalation Schema

| | MGC018 | | | |
|---|---|---|---|---|
| Dose-level | Dose | Lorigerlimab | Dose | Number of |
| Cohort | (mg/kg) | Dose (mg/kg) | Interval | Participants |
| Cohort −1 | 0.5 | 3 | Every 3 weeks | 3 to 6 |
| Cohort 1 | 1.0 | 6 | Every 3 weeks | 3 to 6 |
| Cohort 2 | 2.0 | 6 | Every 3 weeks | 3 to 6 |
| Cohort 3 | 2.7 | 6 | Every 3 weeks | 3 to 6 |
| Cohort 4 | 3.0 | 6 | Every 3 weeks | 3 to 6 |

If it is determined that the MTD for MGC018 and/or lorigerlimab is exceeded in Cohort 1, a dose de-escalation cohort (Cohort −1) may be utilized to evaluate a lower dose of MGC018 (0.5 mg/kg). Additionally, de-escalation to an intermediate dose level of lorigerlimab (3 mg/kg or 1 mg/kg) may be considered.

Both MGC018 and lorigerlimab are administered as a single dose once every 3 weeks. Both MGC018 and lorigerlimab are administered on the same day, with MGC018 administered first followed by administration of lorigerlimab. Each cycle of therapy is defined as 3 weeks, in which MGC018 and lorigerlimab are administered on Day 1 (±3 days). MGC018 will be administered IV over approximately 60 minutes. Lorigerlimab will be administered IV over approximately 30 minutes. Tumor assessments may be performed every 6 weeks (±7 days) for the initial 6 months on treatment, then every 12 weeks (±21 days) until progressive disease (PD). PPK modeling (as in Example 3) may be performed on clinical PK data from the dose escalation trial of MGC018 and/or lorigerlimab to aid in determining the MTD or MAD (if no MTD is defined). Additionally, such modeling may be used to identify intermediate doses of MGC018 and/or lorigerlimab and cohorts at these intermediate doses may be included. For example, the administration of MGC018 at an intermediate dose may be between 2.0 mg/kg and 2.7 mg/kg.

The dose escalation study may be followed by a cohort expansion phase, using the dose of MGC018 established in the dose escalation study, to further define the safety and preliminary antitumor efficacy of the combination in various cancers (Table 7).

TABLE 7
Cohort Expansion Phase Enrollment

| | Total Number of |
|---|---|
| Tumor-specific Cohort | Participants |
| Metastatic castration-resistant prostate cancer | 18 to 40 |
| Melanoma | 14 to 37 |
| Pancreatic cancer | 20 to 42 |
| Hepatocellular carcinoma | 19 to 40 |
| Ovarian cancer | 20 to 41 |
| Renal cell carcinoma | 17 to 42 |
| Total | 108 to 242 |

The dose escalation studies and cohort expansion phases may be adapted to add additional cohorts in which MGC018 and/or lorigerlimab is/are administered as a single dose every 4 weeks (e.g., as shown in Table 8). MGC018 and lorigerlimab may be administered on the same day, with MGC018 administered first followed by administration of lorigerlimab, or with lorigerlimab administered first followed by administration of MGC018. Each cycle of therapy may be defined as 4 weeks, in which MGC018 and lorigerlimab are administered on Day 1 (±3 days). MGC018 will be administered IV over approximately 60 minutes. Lorigerlimab will be administered IV over approximately 30 minutes. As provided above, alternative cohorts may be introduced based for example on PPK modeling data.

TABLE 8
Example Dose Escalation Schema

| | MGC018 | | | |
|---|---|---|---|---|
| Dose-level | Dose | Lorigerlimab | Dose | Number of |
| Cohort | (mg/kg) | Dose (mg/kg) | Interval | Participants |
| Cohort −1 | 0.5 | 3 | Every 4 weeks | 3 to 6 |
| Cohort 1 | 1.0 | 6 | Every 4 weeks | 3 to 6 |
| Cohort 2 | 2.0 | 6 | Every 4 weeks | 3 to 6 |
| Cohort 3\hat{ } | 2.7 | 6 | Every 4 weeks | 3 to 6 |
| Cohort 4 | 3.0 | 6 | Every 4 weeks | 3 to 6 |

All publications and patents mentioned in this specification are herein incorporated by reference to the same extent as if each individual publication or patent application was specifically and individually indicated to be incorporated by reference in its entirety. While the disclosure has been described in connection with specific embodiments thereof, it will be understood that it is capable of further modifications and this application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth.

What is claimed is:

1. A method of treating a cancer in a subject in need thereof, comprising administering an anti-B7-H3 antibody-drug conjugate (B7-H3-ADC) and a PD-1×CTLA-4 bispecific molecule to said subject, wherein said B7-H3-ADC comprises the formula:
Ab-(LM)ₘ-(D)ₙ,
wherein:
Ab is a humanized B7-H3 antibody or B7-H3 binding fragment thereof that binds to B7-H3 and comprises:
(i) the CDRL1 sequence RASESIYSYLA (SEQ ID NO:22), the CDRL2 sequence NTKTLPE (SEQ ID NO:23) and the CDRL3 sequence QHHYGTPPWT (SEQ ID NO:24) in its Variable Light Chain (VL) domain, and
(ii) the CDRH1 sequence SYGMS (SEQ ID NO:25), the CDRH2 sequence TINSGGSNTYY PDSLKG (SEQ ID NO:26) and the CDRH3 sequence HDGGAMDY (SEQ ID NO:27) in its Variable Heavy Chain (VH) domain;
D is a cytotoxic duocarmycin moiety;
LM comprises at least one bond or a Linker Molecule that covalently links Ab and D;
m is an integer between 0 and n and denotes the number of bonds or Linker Molecules of said B7-H3-ADC, except when LM is a bond, m is not 0;
and
n is an integer between 1 and 10 and denotes the number of cytotoxic duocarmycin moieties covalently linked to said B7-H3-ADC molecule.

2. The method of claim 1, wherein said Ab comprises:
(iii) a humanized Variable Light Chain (VL) domain comprising the amino acid sequence of SEQ ID NO:17; and
(iv) a humanized Variable Heavy Chain (VH) domain comprising the amino acid sequence of SEQ ID NO:18.

3. The method of any one of claim 1 or 2, wherein said Ab further comprises an Fc of a human IgG1, IgG2, IgG3, or IgG4.

4. The method of claim 3, wherein said Fc Domain is a variant Fc Domain that comprises:
(a) one or more amino acid modifications that reduces the affinity of the variant Fc Domain for an FcγR; and/or
(b) one or more amino acid modifications that enhances the serum half-life of the variant Fc Domain.

5. The method of any one of claims 1-4, wherein at least one of said LM is a Linker Molecule.

6. The method of any one of claims 1-5, wherein said LM Linker Molecule is a peptidic linker.

7. The method of claim 6, wherein said peptidic linker is a valine-citrulline dipeptide linker.

8. The method of any one of claims 1-7, wherein said LM Linker Molecule further comprises a self-eliminating spacer between the cleavable linker and D.

9. The method of claim 8, wherein said self-eliminating spacer comprises a para-aminobenzyloxycarbonyl moiety.

10. The method of any one of claims 1-9, wherein said LM Linker Molecule further comprises a maleimide linker moiety between the cleavable linker and Ab.

11. The method of any one of claims 1-10, wherein LM is represented by the formula:
[V—(W)ₖ—(X)₁-A]
whereby said B7-H3-ADC is represented by the formula:
Ab-[V—(W)ₖ—(X)₁-A]-D
wherein:
V is a cleavable linker,
(W)ₖ—(X)₁-A is an elongated, self-eliminating spacer system, that self-eliminates via a 1,(4+2n)-elimination,
W and X are each a 1,(4+2n) electronic cascade spacer, being the same or different,
A is either a spacer group of formula (Y)ₘ, wherein Y is a 1,(4+2n) electronic cascade spacer, or a group of formula U, being a cyclisation elimination spacer,
k, l and m are independently an integer of 0 (included) to 5 (included),
n is an integer of 0 (included) to 10 (included),
with the provisos that:
when A is (Y)ₘ: then k+l+m>1, and
if k+l+m=l, then n>l;
when A is U: then k+1≥1.
W, X, and Y are independently selected from compounds having the formula: 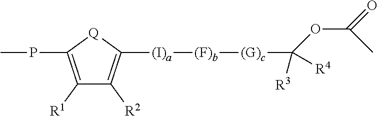
or the formula: 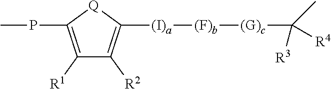
wherein: Q is —R⁵C=CR⁶—, S, O, NR⁵, —R⁵C═N—, or —N═CR⁵—
P is NR⁷, O or S
a, b, and c are independently an integer of 0 (included) to 5 (included);
I, F and G are independently selected from compounds having the formula: 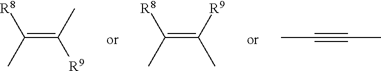
wherein R¹, R², R³, R⁴, R⁵, R⁶, R⁷, R⁸, and R⁹ independently represent H, C₁₋₆ alkyl, C₃₋₂₀ heterocyclyl, C₅₋₂₀ aryl, C₁₋₆ alkoxy, hydroxy (OH), amino (NH₂), mono-substituted amino (NRₓH), di-substituted amino (NRₓ¹Rₓ²), nitro (NO₂), halogen, CF₃, CN, CONH₂, SO₂Me, CONHMe, cyclic C₁₋₅ alkylamino, imidazolyl, C₁₋₆ alkylpiperazinyl, morpholino, thiol (SH), thioether (SRₓ), tetrazole, carboxy (COOH), carboxylate (COORₓ), sulphoxy (S(═O)₂OH), sulphonate (S(═O)₂ORₓ), sulphonyl (S(═O)₂Rₓ), sulphixy (S(═O)OH), sulphinate (S(═O)ORₓ), sulphinyl (S(═O)Rₓ), phosphonooxy (OP(═O)(OH)₂), and phosphate (OP(═O)(ORₓ)₂), where Rₓ, Rₓ¹ and Rₓ² are independently selected from a C₁₋₆ alkyl group, a C₃₋₂₀ heterocyclyl group or a C₅₋₂₀ aryl group, two or more of the substituents R¹, R², R³, R⁴, R⁵, R⁶, R⁷, R⁸, or R⁹ optionally being connected to one another to form one or more aliphatic or aromatic cyclic structures;
U is selected from compounds having the formula: 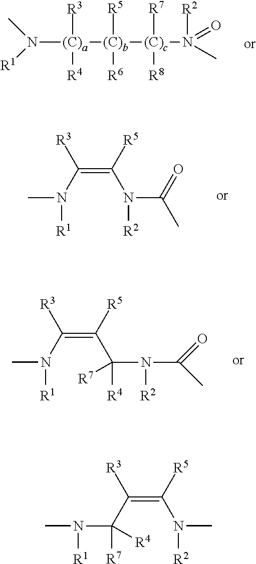
wherein:
a, b and c are independently selected to be an integer of 0 or 1;
provided that a+b+c=2 or 3;
R¹ and/or R² independently represent H, C₁₋₆ alkyl, the alkyl being optionally substituted with one or more of the following groups: hydroxy (OH), ether (ORₓ), amino (NH₂), mono-substituted amino (NRₓH), disubstituted amino (NRₓ¹Rₓ²), nitro (NO₂), halogen, CF₃, CN, CONH₂, SO₂Me, CONHMe, cyclic C₁₋₅ alkylamino, imidazolyl, C₁₋₆ alkylpiperazinyl, morpholino, thiol (SH), thioether (SRₓ), tetrazole, carboxy (COOH), carboxylate (COORₓ), sulphoxy (S(═O)₂OH), sulphonate (S(═O)₂₀Rₓ), sulphonyl (S(═O)₂Rₓ), sulphixy (S(═O)OH), sulphinate (S(═O)ORₓ), sulphinyl (S(═O)Rₓ), phosphonooxy (OP(═O)(OH)₂), and phosphate (OP(═O)(ORₓ)₂), where Rₓ, Rₓ¹ and Rₓ² are selected from a C₁₋₆ alkyl group, a C₃₋₂₀ heterocyclyl group or a C₅₋₂₀ aryl group; and
R³, R⁴, R⁵, R⁶, R⁷ and R⁸ independently represent H, C₁₋₆ alkyl, C₃₋₂₀ heterocyclyl, C₅₋₂₀ aryl, C₁₋₆ alkoxy, hydroxy (OH), amino (NH₂), mono-substituted amino (NRₓH), disubstituted amino (NRₓ¹Rₓ²), nitro (NO₂), halogen, CF₃, CN, CONH₂, SO₂Me, CONHMe, cyclic C₁₋₅ alkylamino, imidazolyl, C₁₋₆ alkylpiperazinyl, morpholino, thiol (SH), thioether (SRₓ), tetrazole, carboxy (COOH), carboxylate (COORₓ), sulphoxy (S(═O)₂OH), sulphonate (S(═O)₂ORₓ), sulphonyl (S(═O)₂Rₓ), sulphixy (S(═O)OH), sulphinate (S(═O)ORₓ), sulphinyl (S(═O)Rₓ), phosphonooxy (OP(═O)(OH)₂), and phosphate (OP(═O)(ORₓ)₂), where Rₓ, Rₓ¹ and Rₓ² are selected from a C₁₋₆ alkyl group, a C₃₋₂₀ heterocyclyl group or a C₅₋₂₀ aryl group, and two or more of the substituents R¹, R², R³, R⁴, R⁵, R⁶, R⁷, or R⁸ are optionally connected to one another to form one or more aliphatic or aromatic cyclic structures.

12. The method of claim 11, wherein said LM linker molecule comprises:
(1) p-aminobenzyloxycarbonyl-p-aminobenzyloxycarbonyl;
(2) p-aminobenzyloxycarbonyl-p-aminobenzyloxycarbonyl-p-aminobenzyloxycarbonyl;
(3) p-ammocinnamyloxycarbonyl;
(4) p-aminocinnamyloxycarbonyl-p-aminobenzyloxycarbonyl;
(5) p-amino-benzyloxycarbonyl-p-aminocinnamyloxycarbonyl;
(6) p-aminocinnamyloxycarbonyl-p-aminocinnamyloxycarbonyl;
(7) p-aminophenylpentadienyloxycarbonyl;
(8) p-aminophenylpentadienyloxycarbonyl-p-aminocinnamyloxycarbonyl;
(9) p-aminophenylpentadienyloxycarbonyl-p-aminobenzyloxycarbonyl;
(10) p-aminophenylpentadienyloxycarbonyl-p-aminophenylpentadienyloxycarbonyl;
(11) p-aminobenzyloxycarbonyl(methylamino)ethyl(methylamino) carbonyl;
(12) p-aminocinnamyloxycarbonyl(methylamino)ethyl(methylamino) carbonyl;
(13) p-aminobenzyloxycarbonyl-p-aminobenzyloxycarbonyl(methylamino) ethyl(methylamino)carbonyl;
(14) p-aminocinnamyloxycarbonyl-p-aminobenzyloxycarbonyl (methylamino)ethyl(methylamino)carbonyl;
(15) p-aminobenzyloxycarbonyl-p-aminocinnamyloxycarbonyl (methylamino)ethyl(methylamino)-carbonyl;
(16) p-aminocinnamyloxycarbonyl-p-aminocinnamyloxycarbonyl (methylamino)ethyl(methylamino)carbonyl;
(17) p-aminobenzyloxycarbonyl-p-aminobenzyl;
(18) p-aminobenzyloxycarbonyl-p-aminobenzyloxycarbonyl-p-aminobenzyl;
(19) p-aminocinnamyl;
(20) p-aminocinnamyloxycarbonyl-p-aminobenzyl;
(21) p-aminobenzyloxycarbonyl-p-aminocinnamyl;
(22) p-amino-cinnamyloxycarbonyl-p-aminocinnamyl;
(23) p-aminophenylpentadienyl;
(24) p-aminophenylpentadienyloxycarbonyl-p-aminocinnamyl;
(25) p-aminophenylpentadienyloxycarbonyl-p-aminobenzyl; or
(26) p-aminophenylpentadienyloxycarbonyl-p-aminophenylpentadienyl.

13. The method of any one of claims 1-12, wherein said LM Linker Molecule is conjugated to the side chain of an amino acid of a polypeptide chain of the Ab and binds the Ab to a molecule of the cytotoxic duocarmycin moiety D.

14. The method of any one of claims 1-13, wherein said cytotoxic duocarmycin moiety D comprises a duocarmycin cytotoxin selected from the group consisting of: duocarmycin A, duocarmycin B1, duocarmycin B2, duocarmycin C1, duocarmycin C2, duocarmycin D, duocarmycin SA, CC-1065, adozelesin, bizelesin, carzelesin (U-80244), seco-duocarmycin (seco-DUBA) and spiro-duocarmycin (spiro-DUBA).

15. The method of claim 14, wherein the cytotoxic duocarmycin moiety D comprises seco-DUBA.

16. The method of any of claims 1-15, wherein said LM Linker Molecule is covalently linked to the Ab via reduced inter-chain disulfides.

17. The method of any one of claims 1-16, wherein said Ab comprises:
(i) a light chain comprising the amino acid sequence of SEQ ID NO:19; and
(ii) a heavy chain comprising the amino acid sequence of SEQ ID NO:20;
said D comprises seco-DUBA; and
said LM comprises a Linker Molecule comprising a maleimide linker moiety, a valine-citrulline dipeptide linker, and a para-aminobenzyloxycarbonyl moiety.

18. The method of any one of claims 1-17, wherein said B7-H3-ADC is administered at a therapeutically effective or prophylactically effective dose of:
a) about 1 mg/kg to about 3 mg/kg every 3 weeks; or
b) about 2 mg/kg to about 3 mg/kg every 3 weeks.

19. The method of any one of claims 1-17, wherein said B7-H3-ADC is administered at a therapeutically effective or prophylactically effective dose of:
a) about 1 mg/kg to about 3 mg/kg every 4 weeks; or
b) about 2 mg/kg to about 3 mg/kg every 4 weeks.

20. The method of any one of claims 1-19, wherein said dose of said B7-H3-ADC is administered as a single dose.

21. The method of any one of claims 1-19, wherein said dose of said B7-H3-ADC is administered as a fractionated dose in two or more separate administrations.

22. The method of any one of claims 1-19 or 21, wherein said fractionated dose comprises two separate administrations administered within a 4-week cycle.

23. The method of any one of claims 1-22, wherein:
(a) said B7-H3-ADC is administered before the administration of said PD-1×CTLA-4 bispecific molecule on days when both said B7-H3-ADC and said PD-1×CTLA-4 bispecific molecule are administered; or
(b) said PD-1×CTLA-4 is administered before the administration of said B7-H3-ADC bispecific molecule on days when both said B7-H3-ADC and said PD-1×CTLA-4 bispecific molecule are administered.

24. The method of claim 23, wherein:
(a) said PD-1×CTLA-4 bispecific molecule is administered at least about 15-30 minutes after said B7-H3-ADC is administered; or
(b) said B7-H3-ADC is administered at least about 15-30 minutes after said PD-1×CTLA-4 bispecific molecule is administered.

25. The method of any one of claims 1-24, wherein said PD-1×CTLA-4 bispecific molecule is selected from the group consisting of: lorigerlimab, MEDI5752, vudalimab, and cadonilimab.

26. The method of any one of claims 1-25, wherein said PD-1×CTLA-4 bispecific molecule is lorigerlimab.

27. The method of claim 26, wherein said lorigerlimab is administered at a dose of about 1 mg/kg, about 3 mg/kg or about 6 mg/kg every 3 weeks.

28. The method of claim 27, wherein said lorigerlimab is administered at a dose of about 6 mg/kg every 3 weeks.

29. The method of claim 26, wherein said lorigerlimab is administered at a dose of about 1 mg/kg, about 3 mg/kg or about 6 mg/kg every 4 weeks.

30. The method of claim 29, wherein said lorigerlimab is administered at a dose of about 6 mg/kg every 4 weeks.

31. The method of any one of claims 1-30, wherein said B7-H3-ADC is administered at a dose of about 1 mg/kg.

32. The method of any one of claims 1-30, wherein said B7-H3-ADC is administered at a dose of about 1.25 mg/kg.

33. The method of any one of claims 1-30, wherein said B7-H3-ADC is administered at a dose of about 1.5 mg/kg.

34. The method of any one of claims 1-30, wherein said B7-H3-ADC is administered at a dose of about 1.75 mg/kg.

35. The method of any one of claims 1-30, wherein said B7-H3-ADC is administered at a dose of about 2 mg/kg.

36. The method of any one of claims 1-30, wherein said B7-H3-ADC is administered at a dose of about 2.1 mg/kg.

37. The method of any one of claims 1-30, wherein said B7-H3-ADC is administered at a dose of about 2.2 mg/kg.

38. The method of any one of claims 1-30, wherein said B7-H3-ADC is administered at a dose of about 2.25 mg/kg.

39. The method of any one of claims 1-30, wherein said B7-H3-ADC is administered at a dose of about 2.3 mg/kg.

40. The method of any one of claims 1-30, wherein said B7-H3-ADC is administered at a dose of about 2.4 mg/kg.

41. The method of any one of claims 1-30, wherein said B7-H3-ADC is administered at a dose of about 2.5 mg/kg.

42. The method of any one of claims 1-30, wherein said B7-H3-ADC is administered at a dose of about 2.6 mg/kg.

43. The method of any one of claims 1-30, wherein said B7-H3-ADC is administered at a dose of about 2.7 mg/kg.

44. The method of any one of claims 1-30, wherein said B7-H3-ADC is administered at a dose of about 2.75 mg/kg.

45. The method of any one of claims 1-30, wherein said B7-H3-ADC is administered at a dose of about 3 mg/kg.

46. The method of any one of claims 1-45, wherein said B7-H3-ADC is administered by intravenous (IV) infusion.

47. The method of claim 46, wherein said IV infusion of said B7-H3-ADC is over a period of at least about 60-120 minutes.

48. The method of claim 47, wherein said IV infusion of said B7-H3-ADC is over a period of at least about 60 minutes.

49. The method of any one of claims 1-48, wherein said PD-1×CTLA-4 bispecific molecule is administered by IV infusion.

50. The method of claim 49, wherein said IV infusion of said PD-1×CTLA-4 bispecific molecule is over a period of at least about 30-120 minutes.

51. The method of claim 50, wherein said IV infusion of said PD-1×CTLA-4 bispecific molecule is over a period of at least about 30 minutes.

52. The method of any one of claims 1-51, wherein said cancer is selected from the group consisting of: an adrenal gland cancer, an AIDS-associated cancer, an alveolar soft part sarcoma, an astrocytic tumor, an anal cancer, squamous cell carcinoma of the anal canal (SCAC), a bladder cancer, a bone cancer, a brain and spinal cord cancer, a metastatic brain tumor, a B-cell cancer, a breast cancer, a HER2+ breast cancer, triple negative breast cancer (TNBC), a carotid body tumors, a cervical cancer, a chondrosarcoma, a chordoma, a chromophobe renal cell carcinoma, a clear cell carcinoma, a colon cancer, a colorectal cancer (CRC), a non-microsatellite instability high colorectal cancer (non-MSI-H CRC), a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, a gastric cancer, a gestational trophoblastic disease, a germ cell tumor, a head and neck cancer, a glioblastoma, a hematological malignancy, a hepatocellular carcinoma, an islet cell tumor, a Kaposi's Sarcoma, a kidney cancer, a leukemia, an acute myeloid leukemia, a liposarcoma/malignant lipomatous tumor, a dedifferentiated liposarcoma, a liver cancer, a lymphoma, a lung cancer, a non-small-cell lung cancer (NSCLC), a medulloblastoma, a melanoma, a cutaneous melanoma, a meningioma, a mesothelioma pharyngeal cancer, a multiple endocrine neoplasia, a multiple myeloma, a myelodysplastic syndrome, a myxofibrosarcoma, a neuroblastoma, a neuroendocrine tumors, an ovarian cancer, a pancreatic cancer, a papillary thyroid carcinoma, a parathyroid tumor, a pediatric cancer, a peripheral nerve sheath tumor, a phaeochromocytoma, a pituitary tumor, a prostate cancer, a metastatic castration resistant prostate cancer (mCRPC), a posterior uveal melanoma, a renal cell cancer, a renal cell carcinoma (RCC), a renal metastatic cancer, a rhabdoid tumor, a rhabdomyosarcoma, a sarcoma, a skin cancer, a small round blue cell tumor of childhood, a neuroblastoma, a soft tissue sarcoma, a pleomorphic undifferentiated sarcoma, a squamous cell cancer, a squamous cell cancer of the head and neck (SCCHN), a stomach cancer, a synovial sarcoma, a testicular cancer, a thymic carcinoma, a thymoma, a thyroid cancer, a thyroid metastatic cancer, and a uterine cancer.

53. The method of claim 52, wherein said cancer is selected from the group consisting of: anal cancer, SCAC, breast cancer, TNBC, cervical cancer, colorectal cancer, non-microsatellite instability high colorectal cancer (non-MSI-H CRC), head and neck cancer, kidney cancer, renal cell carcinoma, liver cancer, hepatocellular carcinoma, lung cancer, NSCLC, melanoma, cutaneous melanoma, posterior uveal melanoma, ovarian cancer, pancreatic cancer, prostate cancer, mCRPC, soft tissue sarcoma, dedifferentiated liposarcoma, myxofibrosarcoma, pleomorphic undifferentiated sarcoma, synovial sarcoma, squamous cell cancer, and SCCHN.

54. The method of any one of claims 1-53, wherein said cancer is prostate cancer.

55. The method of any one of claims 52-54, wherein said prostate cancer is mCRPC.

56. The method of any one of claims 1-53, wherein said cancer is liver cancer.

57. The method of any one of claims 52, 53, or 56, wherein said liver cancer is hepatocellular carcinoma.

58. The method of any one of claims 1-53, wherein said cancer is kidney cancer.

59. The method of any one of claims 52, 53, or 58, where said kidney cancer is renal cell carcinoma.

60. The method of any one of claims 1-53, wherein said cancer is ovarian cancer.

61. The method of any one of claims 1-53, where said cancer is pancreatic cancer.

62. The method of any one of claims 1-53, wherein said cancer is anal cancer.

63. The method of any one of claims 52, 53, or 62, wherein said anal cancer is SCAC.

64. The method of any one of claims 1-53, wherein said cancer is a squamous cell cancer.

65. The method of any one of claims 52, 53, or 64, wherein said squamous cell cancer is SCCHN.

66. The method of any one of claims 1-53, wherein said cancer is breast cancer.

67. The method of any one of claims 52, 53, or 66, wherein said breast cancer is TNBC.

68. The method of any one of claims 1-53, wherein said cancer is melanoma.

69. The method of any one of claims 52, 53, or 68, wherein said melanoma is a cutaneous melanoma or a posterior uveal melanoma.

70. The method of any one of claims 1-53, wherein said cancer is lung cancer.

71. The method of any one of claims 52, 53, or 70, wherein said lung cancer is NSCLC.

72. The method of any one of claims 1-53, wherein said cancer is cervical cancer.

73. The method of any one of claims 1-53, wherein said cancer is colorectal cancer.

74. The method of any one of claims 52, 53, or 73, wherein said colorectal cancer is non-MSI-H CRC.

75. The method of any one of claims 1-53, wherein said cancer is a soft tissue sarcoma.

76. The method of any one of claims 52, 53, or 75, wherein said soft tissue sarcoma is dedifferentiated liposarcoma, myxofibrosarcoma, pleomorphic undifferentiated sarcoma, or synovial sarcoma.

77. The method of any one of claims 1-76, wherein said cancer expresses B7-H3.

78. The method of any one of claims 1-77, further comprising administering a therapeutically or prophylactically effective amount of one or more additional therapeutic agents or chemotherapeutic agents.

79. The method of claim 78, wherein said chemotherapeutic agent is a platinum-based chemotherapeutic agent.

80. The method of claim 78, wherein said chemotherapeutic agent is a taxane.

81. The method of any one of claims 1-80, wherein said subject in need thereof is a human.


SEQUENCE LISTING (38 sequences)

SEQ ID NO: 1 - protein, 107 aa, Homo sapiens [sequence omitted]

SEQ ID NO: 2 - protein, 104 aa, Homo sapiens
  REGION 1..104: note = misc_feature - amino acid sequence of a human CL Lambda Domain
QPKAAPSVTLFPPSSEELQANKATLVCLISDFYPGAVTVAWKADSSPVKAGVETTPSKQSNNKYAASSYLSLTPEQWKSHRSYSCQVTHEGSTVEKTVAPTECS

SEQ ID NO: 3 - protein, 98 aa, Homo sapiens
  REGION 1..98: note = misc_feature - amino acid sequence of a human IgG1 CH1 Domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 4 - protein, 98 aa, Homo sapiens
  REGION 1..98: note = misc_feature - amino acid sequence of a human IgG4 CH1 Domain
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 5 - protein, 15 aa, Homo sapiens
  REGION 1..15: note = misc_feature - amino acid sequence of a human IgG1 Hinge Domain
EPKSCDKTHTCPPCP

SEQ ID NO: 6 - protein, 12 aa, Homo sapiens
  REGION 1..12: note = misc_feature - amino acid sequence of a human IgG4 Hinge Domain
ESKYGPPCPSCP

SEQ ID NO: 7 - protein, 12 aa, synthetic construct
  REGION 1..12: note = Synthetic: amino acid sequence of a S228P-stabilized human IgG4 Hinge Domain
ESKYGPPCPPCP

SEQ ID NO: 8 - protein, 217 aa, synthetic construct
  REGION 1..217: note = Synthetic: amino acid sequence of the CH2-CH3 Domain of a representative human IgG1
  VARIANT 217: note = X is a lysine (K) or is absent
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 9 - protein, 217 aa, synthetic construct
  REGION 1..217: note = Synthetic: amino acid sequence of the CH2-CH3 Domain of a representative human IgG4
  VARIANT 217: note = X is a lysine (K) or is absent
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGX

SEQ ID NO: 10 - protein, 8 aa, synthetic construct
  REGION 1..8: note = Synthetic: intervening linker peptide Linker 1
GGGSGGGG

SEQ ID NO: 11 - protein, 6 aa, synthetic construct
  REGION 1..6: note = Synthetic: cysteine-containing intervening linker peptide Linker 2
GGCGGG

SEQ ID NO: 12 - protein, 28 aa, synthetic construct
  REGION 1..28: note = Synthetic: cysteine-containing Heterodimer-Promoting (E-coil) Domain
EVAACEKEVAALEKEVAALEKEVAALEK

SEQ ID NO: 13 - protein, 28 aa, synthetic construct
  REGION 1..28: note = Synthetic: cysteine-containing Heterodimer-Promoting (K-coil) Domain
KVAACKEKVAALKEKVAALKEKVAALKE

SEQ ID NO: 14 - protein, 217 aa, synthetic construct
  REGION 1..217: note = Synthetic: variant IgG4 sequence for the CH2 and CH3 Domains comprising the M252Y/S254T/T256E substitutions
  VARIANT 217: note = X is a lysine (K) or is absent
APEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGX

SEQ ID NO: 15 - protein, 108 aa, synthetic construct
  REGION 1..108: note = Synthetic: amino acid sequence of the VL Domain of the murine anti-B7-H3 antibody mAb-A
DIQMTQSPASLSVSVGETVTITCRASESIYSYLAWYQQKQGKSPQLLVYNTKTLPEGVPSRFSGSGSGTQFSLKINSLQPEDFGRYYCQHHYGTPPWTFGGGTNLEIK

SEQ ID NO: 16 - protein, 117 aa, synthetic construct
  REGION 1..117: note = Synthetic: amino acid sequence of the VH Domain of anti-B7-H3 mAb-A
EVQQVESGGDLVKPGGSLKLSCAASGFTFSSYGMSWVRQTPDKRLEWVATINSGGSNTYYPDSLKGRFTISRDNAKNTLYLQMRSLKSEDTAMYYCARHDGGAMDYWGQGTSVTVSS

SEQ ID NO: 17 - protein, 108 aa, synthetic construct
  REGION 1..108: note = Synthetic: amino acid sequence of the VL Domain of hmAb-A
DIQMTQSPSSLSASVGDRVTITCRASESIYSYLAWYQQKPGKAPKLLVYNTKTLPEGVPSRFSGSGSGTDFTLTISSLQPEDFATYYCQHHYGTPPWTFGQGTRLEIK

SEQ ID NO: 18 - protein, 117 aa, synthetic construct
  REGION 1..117: note = Synthetic: amino acid sequence of the VH Domain of hmAb-A
EVQLVESGGGLVKPGGSLRLSCAASGFTFSSYGMSWVRQAPGKGLEWVATINSGGSNTYYPDSLKGRFTISRDNAKNSLYLQMNSLRAEDTAVYYCARHDGGAMDYWGQGTTVTVSS

SEQ ID NO: 19 - protein, 215 aa, synthetic construct
  REGION 1..215: note = Synthetic: amino acid sequence of a Light Chain of hmAb-A comprising the VL of Domain of hmAb-A and a CL Kappa Domain
DIQMTQSPSSLSASVGDRVTITCRASESIYSYLAWYQQKPGKAPKLLVYNTKTLPEGVPSRFSGSGSGTDFTLTISSLQPEDFATYYCQHHYGTPPWTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 447 aa, synthetic construct
  REGION 1..447: note = Synthetic: amino acid sequence of a Heavy Chain comprising the VH Domain of hmAb-A and IgG1 CH1-H-CH2-CH3 Domains
  VARIANT 447: note = X is a lysine (K) or is absent
EVQLVESGGGLVKPGGSLRLSCAASGFTFSSYGMSWVRQAPGKGLEWVATINSGGSNTYYPDSLKGRFTISRDNAKNSLYLQMNSLRAEDTAVYYCARHDGGAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 21 - protein, 4 aa, synthetic construct
  REGION 1..4: note = Synthetic: Tetrapeptide
LLQL

SEQ ID NO: 22 - protein, 11 aa, synthetic construct
  REGION 1..11: note = Synthetic: CDRL1 sequence
RASESIYSYLA

SEQ ID NO: 23 - protein, 7 aa, synthetic construct
  REGION 1..7: note = Synthetic: CDRL2 sequence
NTKTLPE

SEQ ID NO: 24 - protein, 10 aa, synthetic construct
  REGION 1..10: note = Synthetic: CDRL3 sequence
QHHYGTPPWT

SEQ ID NO: 25 - protein, 5 aa, synthetic construct
  REGION 1..5: note = Synthetic: CDRH1 sequence
SYGMS

SEQ ID NO: 26 - protein, 17 aa, synthetic construct
  REGION 1..17: note = Synthetic: CDRH2 sequence
TINSGGSNTYYPDSLKG

SEQ ID NO: 27 - protein, 8 aa, synthetic construct
  REGION 1..8: note = Synthetic: CDRH3 sequence
HDGGAMDY

SEQ ID NO: 28 - protein, 499 aa, synthetic construct
  REGION 1..499: note = Synthetic: amino acid sequence of the first and third polypeptide chains of lorgerlimab
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKGGGSGGGGQVQLVESGGGVVQPGRSLRLSCAASGFTFSSYTMHWVRQAPGKGLEWVTFISYDGSNKHYADSVKGRFTVSRDNSKNTLYLQMNSLRAEDTAIYYCARTGWLGPFDYWGQGTLVTVSSGGCGGGEVAACEKEVAALEKEVAALEKEVAALEKESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 29 - protein, 269 aa, synthetic construct
  REGION 1..269: note = Synthetic: amino acid sequence of the second and fourth polypeptide chains of lorigerlimab
EIVLTQSPGTLSLSPGERATLSCRASQSVSSSFLAWYQQKPGQAPRLLIYGASSRATGIPDRFSGSGSGTDFTLTISRLEPEDFAVYYCQQYGSSPWTFGQGTKVEIKGGGSGGGGQVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSGGCGGGKVAACKEKVAALKEKVAALKEKVAALKE

SEQ ID NO: 30 - protein, 111 aa, synthetic construct
  REGION 1..111: note = Synthetic: VL Domain of a monoclonal antibody capable of binding to PD-1
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIK

SEQ ID NO: 31 - protein, 119 aa, synthetic construct
  REGION 1..119: note = Synthetic: VH Domain of a monoclonal antibody capable of binding PD-1
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSS

SEQ ID NO: 32 - protein, 108 aa, synthetic construct
  REGION 1..108: note = Synthetic: VL Domain of a monoclonal antibody capable of binding to CTLA-4
EIVLTQSPGTLSLSPGERATLSCRASQSVSSSFLAWYQQKPGQAPRLLIYGASSRATGIPDRFSGSGSGTDFTLTISRLEPEDFAVYYCQQYGSSPWTFGQGTKVEIK

SEQ ID NO: 33 - protein, 118 aa, synthetic construct
  REGION 1..118: note = Synthetic: VH Domain of a monoclonal antibody capable of binding to CTLA-4
QVQLVESGGGVVQPGRSLRLSCAASGFTFSSYTMHWVRQAPGKGLEWVTFISYDGSNKHYADSVKGRFTVSRDNSKNTLYLQMNSLRAEDTAIYYCARTGWLGPFDYWGQGTLVTVSS

SEQ ID NO: 34 - protein, 447 aa, synthetic construct
  REGION 1..447: note = Synthetic: amino acid sequence of the first chain (heavy chain; anti-CTLA-4) of vudalimab
EVQLVESGGGLVKPGGSLRLSCAASGFTFSSYTMHWVRQAPGKGLEWVSFISYDGNNKYYADSVKGRFTISRDNAKNSLYLQMNSLRAEDTAVYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 35 - protein, 215 aa, synthetic construct
  REGION 1..215: note = Synthetic: amino acid sequence of the second chain (light chain; anti-CTLA-4) of vudalimab
EIVLTQSPGTLSLSPGERATLSCRASQSVSSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 480 aa, synthetic construct
  REGION 1..480: note = Synthetic: amino acid sequence of the third chain (scFv-h-CH2-CH3; anti-PD-1) of vudalimab
EIVLTQSPATLSASPGERVTLTCRASQSVGNDVAWYQQKPGQAPRLLINYASHRYTGVPDRFTGSGYGTEFTLTISSVQSEDFGVYYCQQDFSSPRTFGGGTKVEIKGKPGSGKPGSGKPGSGKPGSEVQLVESGGGLVKPGGSLRLSCVASGFTFSNYWMNWVRQAPGKGLEWVAEIRLYSNNYATHYAESVKGRFTISRDDSKSTLYLQMNNLKTEDTGVYYCTRYYGNYGGYFDVWGRGTLVTVSSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 37 - protein, 713 aa, synthetic construct
  REGION 1..713: note = Synthetic: amino acid sequence of the first chain (heavy chain; anti-PD-1 and anti-CTLA-4 (scFv)) of cadonilimab
EVQLVESGGGLVQPGGSLRLSCAASGFAFSSYDMSWVRQAPGKGLDWVATISGGGRYTYYPDSVKGRFTISRDNSKNNLYLQMNSLRAEDTALYYCANRYGEAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVESGAEVKKPGASVKVSCKASGYSFTGYTMNWVRQAPGQCLEWIGLINPYNNITNYAQKFQGRVTFTVDTSISTAYMELSRLRSDDTGVYFCARLDYRSYWGQGTLVTVSAGGGGSGGGGSGGGGSGGGGSQAVVTQEPSLTVSPGGTVTLTCGSSTGAVTTSNFPNWVQQKPGQAPRSLIGGTNNKASWTPARFSGSLLGGKAALTISGAQPEDEAEYYCALWYSNHWVFGCGTKLTVLR

SEQ ID NO: 38 - protein, 214 aa, synthetic construct
  REGION 1..214: note = Synthetic: amino acid sequence of the second chain (light chain; anti-PD-1) of cadonilimab
DIQMTQSPSSMSASVGDRVTFTCRASQDINTYLSWFQQKPGKSPKTLIYRANRLVSGVPSRFSGSGSGQDYTLTISSLQPEDMATYYCLQYDEFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC